(19) United States
(12) Patent Application Publication
(10) Pub. No.: US 2009/0220436 A1
(43) Pub. Date: Sep. 3, 2009

(54) IN SITU POLYMERIZATION FOR SKIN TREATMENT

(75) Inventors: Daniel Griffith Anderson, Sudbury, MA (US); David Thomas Puerta, Melrose, MA (US); Betty Yu, Cambridge, MA (US); Susan Eilidh Bedford, Carlisle, MA (US)

Correspondence Address: FITZPATRICK CELLA HARPER & SCINTO, 30 ROCKEFELLER PLAZA, NEW YORK, NY, 10112, US

(73) Assignee: LIVING PROOF, INC., Cambridge, MA (US)

(21) Appl. No.: 12/300,451

(22) PCT Filed: May 11, 2007
(86) PCT No.: PCT/US07/68751

§ 371 Date: Jan. 26, 2009

Related U.S. Application Data
(60) Provisional application No. 60/800,142, filed on May 11, 2006.
(60) Provisional application No. 60/800,143, filed on May 11, 2006.

(51) Int. Cl. A61K 9/12; A61K 8/00; A61Q 17/04; A61K 47/08; A61K 47/14

(52) U.S. Cl. 424/45; 424/59; 424/63; 514/772; 514/785

(57) ABSTRACT

Cosmetics represent a world-wide multi-billion dollar industry. The present invention provides technology for polymerizing monomers on skin in situ to produce desired skin characteristics. For example, the inventive system may be used to reduce the signs of aging or to protect the treated skin from UV light. The polymerization of monomers on skin is typically initiated using a thermal or photoinitiatied free radical initiator. In certain embodiments, the monomers are fluorinated thereby producing a fluorinated polymer on the skin upon polymerization. The invention provides monomers, initiators, methods, and kits for use in treating skin with polymers.

### RELATED APPLICATIONS

The present application claims priority under 35 U.S.C. § 119(e) to U.S. provisional patent applications, U.S. Ser. No. 60/800,142, filed May 11, 2006; and U.S. Ser. No. 60/800,143, filed May 11, 2006; the entire contents of each of which is incorporated herein by reference.

### BACKGROUND OF THE INVENTION

Cosmetics or makeup are substances which are generally used to enhance the beauty of the human body, apart from simple cleaning. Their use is widespread and the cosmetic industry is very profitable worldwide. The various forms of cosmetics include: lipstick, lip gloss, foundation and powder, rouge, mascara, eyeliner, eyeshadow, nail polish, concealer, creams, and lotions.

The skin is the largest organ of the human body and extends over the entire body. The skin functions primarily to protect us from the outside world. The skin also functions to regulate the temperature of the body, protects the body from harmful UV rays, provides a defenses against pathogens, stores fat, provides the sense the touch, excretes waste, synthesizes vitamin D, and provides cushioning and attachment. In protecting us from the outside world, the skin is constantly exposed to harsh temperatures, sunlight, dirt, dust, wind, chemicals, pathogens, and other insults. In addition, the skin is routinely subjected to washing, shaving, and the application of cosmetics, pharmaceutical agents (e.g. steroids, antibiotics, etc.), and other skin care products.

Skin is composed of two major layers: the epidermis and the underlying dermis, which are distinct in terms of their architecture, physiology, and function. The epidermis is a stratified epithelium composed of four layers: the stratum basale, stratum spinosum, stratum granulosum, and the outermost stratum corneum. The stratum basale contains a single layer of cuboidal keratinocytes attached to a basement membrane. Above this layer is the spinous layer, characterized by presence of numerous desmosomes. The stratum granulosum overlies the stratum spinosum and consists of keratinocytes that contain basophilic granules of keratohyalin as well as lamellar granules in the intercellular compartment. The stratum corneum is the most superficial layer and is composed of anucleated, flattened, fully keratinized cells (corneocytes) fused together to form a plate-like structure. The intercellular space is occupied by ordered lipid lamellae that contain specialized proteins and lipids, such as ceramides, fatty acids, and cholesterols, which are secreted from lamellar bodies in the stratum granulosum. The resulting “bricks and mortar” structure provides the stratum corneum with the ability to perform its protective and moisture retaining functions. The thickness of the epidermis ranges from about 75 to 150 μm except on the soles and palms, where it is about 0.4 to 0.6 mm. The dermoepidermal junction (DEJ) is an undulating basement membrane composed primarily of collagen that separates the epidermis from the dermis.

The dermis is a dense, fibroelastic connective tissue that lies beneath the epidermis and provides a strong and flexible supporting layer. It is composed of cells (e.g. fibroblasts), ground substance, and a fibrous network containing collagenous and elastic fibers and also contains blood vessels, nerves, hair follicles, smooth muscle, glands and lymphatic tissue. Collagen, primarily types I, III, V, and VI, forms the majority of the fibrous component, making up about 75% of the dry weight of the dermis and imparting firmness and tensile strength.

The dermis can be divided into two regions. The papillary dermis conforms to the shape of the overlying epidermis. The reticular dermis lies below the papillary dermis and forms the majority of the dermal layer, giving it most of its elasticity and strength. Elastic fibers of the papillary dermis are oriented parallel (elaunin fibers) or perpendicular (oxytalan fibers) to the DEJ and are thinner than the elastic fibers of the reticular dermis. Oxytalan fibers lack the elastin core while elaunin fibers contain a small amount of elastin. Mature elastin fibers are found primarily arranged in bundles in the reticular dermis and measure about 1-3 μm in diameter.

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological ageing and other biochemical changes from within the skin. Whether extrinsic or intrinsic, these factors result in visible signs of skin ageing and environmental damage, such as wrinkling and other forms of roughness (including increased pore size, flaking, and skin lines), and other histological changes associated with skin ageing or damage. To many people, skin wrinkles are a reminder of the disappearance of youth. As a result, the elimination of wrinkles has become a booming business in our youth-conscious society. Treatments range from cosmetic creams and moisturizers to various forms of cosmetic surgery.

Much research has been focused on developing products which help improve the appearance of skin by attempting to reverse the signs of ageing skin or by covering up imperfections which appear with ageing. Current products treat already aged skin using a variety of techniques including applying moisturizers to the skin, microderm abrasion to remove the outer, lifeless layers of skin, and the application of collagen- and vitamin-containing compositions to help tighten loose or wrinkled skin. In addition, products have been developed which physically cover up skin imperfections while matching skin tone and color or which use chemical compounds to refract or disperse light at problem spots. While such approaches have proven to be effective in reducing the appearance of aged skin, problems common to many of these products include inconvenience due to accidental smearing or wiping of the applied product, the need for daily application, and poor longevity manifested by cracking or flaking.

### SUMMARY OF THE INVENTION

As described herein, it has been discovered that polymers generated via in situ polymerization on skin produce effects and characteristics desired by skin product consumers. Using the appropriate monomer with an optional polymerization initiator, a polymer can be created on the skin upon application of light or heat. Heat is particularly preferred as the activator; however, light which is not damaging to skin (e.g., higher wavelength and/or lower intensity) may also be used to initiate polymerization. The resulting treatment is longer lasting than treatments based on pre-formed polymers and may resist humidity, washing, wiping, flaking, and other deterioration of the desired cosmetic effectiveness. The result is longer lasting cosmetics. The inventive treatment may also be useful in exfoliation treatments such as skin peels.

Based on the in situ polymerization technique a whole new class of polymers can be used in skin care that could not be used before. For example, hydrophobic polymers that are difficult to solubilize in conventional skin care product formulations can now be used in skin treatments. Polymerization in situ on skin provides a treatment that is robust and is effective for longer periods of time and in more demanding environments than conventional skin care products formulated using pre-formed polymers. The inventive treatment may last from several days, to weeks, to months. In addition, it has been found that such polymers generated in situ on skin are able to exhibit longer lasting cosmetic effects. The inventive skin treatment may also provide a more uniform film on the user's skin resulting in a better appearance than traditional cosmetics.

The present invention relates to a system for the in situ polymerization of polymerizable monomers on skin. The treatment may be used to reduce the appearance of wrinkles and ageing. The treatment may also be used to protect the skin from UV light. The treatment may also be used to impart upon the treated skin a desired characteristic such as a desired feel and/or appearance. For example, the treatment may be used to change the color of the skin or make more uniform the pigmentation or color of the skin. The present invention utilizes a novel method of polymerizing monomers directly on skin via a conditionally initiated in situ polyermization process. For example, the polymerization may be initiated by heat or light. The in situ polymerization process allows for the development and use of polymers that could not be used easily or effectively in skin treatment applications in the pre-formed state.

In one aspect, the invention provides a method for treating skin comprising applying to the skin of a subject at least one polymerizable monomer and, optionally, at least one polymerization initiator, and initiating polymerization, thereby causing the polymerization of the polymerizable monomers on the skin. In certain embodiments, two or more different polymerizable monomers may be used in the treatment. The polymerization is typically a free radical polymerization, which is heat initiated or photoinitiated. The type of initiation used may depend on the monomers and/or initiators being used in the treatment. The polymer may bond to the skin during the polymerization process. For example, the polymer may bond with the keratin, collagen, elastin, or other biomolecules found in skin. The polymer film prepared in this manner is preferably resistant to humidity, washing, smearing, wiping, flaking, and/or other deterioration of the desired cosmetic effect.

In certain embodiments, the invention provides a method for treating skin comprising applying to the skin of a subject a composition comprising at least one polymerizable monomer, at least one polymerization initiator, and, optionally, an acceptable solvent or other excipient (e.g., a physiologically, cosmetically, or pharmaceutically acceptable solvent or other excipient), and initiating polymerization, thereby causing the polymerization of the monomers on the skin. In certain embodiments, at least two different monomers are used. The monomers may be provided in the same or different compositions with or without a polymerization initiator. The composition containing monomer typically contains a polymerization initiator, though the initiator can also be applied in a separate treatment step. The composition(s) can be applied by spraying, brushing, rubbing, smearing, rolling-on, immersing, dipping, spattering, pouring, etc. onto the subject's skin. In certain embodiments, the resulting polymer formed on the skin is resistant to the rapid smearing, removal, flaking, or degradation of traditional skin product that contain pre-formed polymers. In certain embodiments, the monomers comprise about 0.1% to about 50% by weight of the composition. In certain embodiments, the monomers comprise about 0.1% to about 20% by weight. In certain embodiments, the monomers comprise about 0.5% to about 10% by weight. In certain embodiments, the monomers comprise about 0.5% to about 5% by weight. In certain embodiments, the monomers comprise about 1%, about 2%, about 3%, about 4%, or about 5% by weight of the composition. The solvent or other excipient then make up the remainder of the composition. Typically, the solvent or other non-active ingredients make up from about 90% to about 99% of the composition. Typically, when the polymerization process is photoinitiated lower concentrations of the polymerizable monomer in the composition are needed, for example, from about 0.1% to about 5%. When the polymerization process is heat initiated, high concentrations of monomer may be used. In certain embodiments, the polymerizable monomers comprises up to about 50% of the composition for heat-activated polymerization processes. The concentration of monomer in the composition affects the overall strength and durability of the resulting polymer. Embodiments with high concentrations of monomer are effective in generating stronger polymers. Embodiments with lower concentrations of monomer are effective in generating polymers that are easier to manipulate. The polymerization initiator comprises about 0.1% to about 10% by weight, or about 0.5% to about 5% by weight of the composition. In certain embodiments, the polymerization initiator is about 1%, about 2%, about 3%, about 4%, or about 5% by weight. The solvent or other excipient then make up the remainder of the composition. Typically, the solvent or other non-active ingredients make up from about 90% to about 99% of the composition. Suitable solvents include water, alcohols (e.g., denatured ethanol, ethanol, isopropanol), propylene glycol, ethylene glycol, and combinations thereof. The solvent may be a propellant such as difluoroethane or dimethyl ether. Preferably the components of the compositions are all biocompatible and do not cause undesired side effects such as inflammation, allergic reactions, etc. The compositions useful in treating skin in accordance with the present invention are also considered to be part of the present invention. For example, compositions comprising monomers, a polymerization initiator, and optionally, a suitable solvent or other excipient are provided by the present invention.

In certain embodiments, the polymerization initiator is activated by irradiation with light. In certain embodiments, the light used is IR, visible, or UV light. In certain embodiments, the UV light use has a wavelength of from about 200 nm to about 600 nm. In certain embodiments, the UV light has a wavelength of from about 200 nm to about 400 nm. In certain embodiments, the wavelength of the UV light is about 365 nm. In certain embodiments, the intensity of the light is from about 500 μW/cm² to about 10,000 μW/cm². In certain particular embodiments, the intensity of the light is about 7,000 μW/cm². The light may be applied to the skin as the monomer and initiator is being applied or subsequent to the application of the monomer and initiator to the skin. Treated skin is exposed to the appropriate light for about 10 seconds to about 1 minute, preferably, from about 20 seconds to about 40 seconds.

In certain other embodiments, the polymerization initiator is activated by exposing the skin to heat. The heat may be applied via a blow dryer, heat lamps, hair dryer, or other devices suitable for delivering heat to skin. The temperatures needed to initiate heat range from about 30° C. to about 120° C. The output temperature of the heat source is typically in the range of about 50° C. to about 500° C. In certain embodiments, the output temperature of the heat source is from about 50° C. to about 200° C. Treated skin is exposed to the heat source for about 10 seconds to about 2 minutes, preferably, from about 20 seconds to about 60 seconds. Enough heat is used to polymerize the monomer but to prevent damage, such as irritation or burning of the skin.

In certain embodiments, the polymerizable monomers used in the present invention include compounds with unsaturated functional groups (e.g. alkenes, alkynes, carbonyls), halogenated compounds, or other compounds with activated functional groups (e.g., epoxides). In certain embodiments, the monomer comprises a vinyl moiety, an acrylate or methacrylate moiety, a diene moiety, a maleimide moiety, or an epoxy moiety. Certain exemplary monomers useful in accordance with the present invention include ethyl acrylate, vinyl acrylate, 1,3-butanediol diacrylate, dipentaerythritol pentaacrylate, tridecyl methacrylate, styrene, and 3,4-epoxycyclohexylmethyl 3′,4′-epoxycyclohexane carboxylate. Various molecular weights of the oligomer may be used.

In certain embodiments, the present invention provides a system for polymerizing fluorinated monomers on skin. Fluorinated monomers have been chosen for use in skin care due to the unique properties of the resulting fluorinated polymers. While preformed fluorinated polymers are not good candidates for traditional skin care products due to their low solubility and unfavorable surface tension, polymerization of fluorinated monomers on the skin surface overcomes these drawbacks and imparts unique and desirable properties to the skin. For example, the in situ polymerization of fluorinated monomers on skin results in skin with an improved appearance, as well as a distinct feel. In certain embodiments, the invention provides a method for polymerizing fluorinated monomers on skin.

Any non-toxic fluorinated monomer suitable for polymerization may be used in the inventive skin treatment. Examples of suitable monomers include alkenes, alkynes, acrylates, methacrylates, fluoroacrylates, or other functional groups with an unsaturated functional group. The fluorinated monomer can include any number of fluorine atoms. In certain embodiments, the fluorinated monomer contains at least one fluorine atom. In certain other embodiments, the fluorinated monomer contains at least two, three, four, five, ten, fifteen, or twenty fluorine atoms. In certain embodiments, at least 10%, 25%, 30%, 40%, 50%, 60%, 75%, 80%, 90%, or 95% of the total number of hydrogen and fluorine atoms in the monomer are fluorine atoms. The monomer may also contain functional groups that are perfluorinated (e.g., an alkyl group). The fluorinated monomer may be mixed with unfluorinated monomers so that a co-polymer is formed upon polymerization.

In certain embodiments, the polymerization initiator is a free radical initiator. In certain embodiments, the polymerization initiator is oxygen-tolerant. In other embodiments, the polymerization initiator is a thermal initiator. In certain embodiments, the free radical initiator is selected from the group consisting of benzophenone, benzyl dimethyl ketal, trimethylphosphine oxides, methyl thio phenyl morphlino ketones. In certain embodiments, the polymerization initiator is a cationic radical initiator such diaryliodonium and triarylsulfonium salts (e.g. benzoyl peroxide, 2,2′-azo-bis-isobutyrylnitrile (AIBN)).

The polymerization process is performed under conditions suitable to yield the desired properties of the resulting polymer. For example, the extent of polymerization or cross-linking may be controlled by the time of the reaction, the amount/concentration of initiator, the polymer starting material, the initiator, the frequency of the light used, additives, temperature of the reaction, solvent used, concentration of polymer starting material, oxygen inhibition, water or solvent inhibition, etc.

The inventive polymer system can be used in a variety of skin care treatments. The inventive treatment may affect the color, condition, appearance, strength, elasticity, smoothness, and/or optical properties of the treated skin. The inventive system can also be used to treat damaged skin. The inventive system may also be used to protect the skin from UV light.

In another aspect, the invention provides kits for treating skin based on polymerizing monomers on skin in situ. The kit typically contains all the materials needed for treating skin using the inventive system. Materials in the kit may include all or some of the following: monomer(s) (e.g., fluorinated monomers, non-fluorinated monomers), polymerization initiator, solvent, excipients, water, applicator, spray bottle, brush, bottle, light source, heat source, instructions for use, etc. In certain embodiments, the kit includes the monomers needed for the skin treatment, the polymerization initiator, and the solvent or other acceptable excipients useful in the inventive skin treatment system. The kit may include the materials conveniently packaged for use in a cosmetician's shop or for home use. The kit typically includes instructions for teaching one how to use the components of the kit in treating skin. The kit may include the materials needed for a single use or for multiple uses.

The present invention does not include the use of the inventive in situ polymerization system to hold human tissue together. That is, the invention does not include using in situ polymerization to close wounds, stop bleeding, or promote wound healing using surgical glues and adhesives (e.g., cyanoacrylate-based adhesives). The invention also does not include the use of the inventive system for the delivery of a pharmaceutical agent, for example, transdermal, intradermal, or subcutaneous delivery of pharmaceutical agents.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that the polymers, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term acyl as used herein refers to a group having the general formula —C(═O)R, where R is alkyl, alkenyl, alkynyl, aryl, carbocylic, heterocyclic, or aromatic heterocyclic. An example of an acyl group is acetyl.

The term aliphatic, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-10 carbon atoms. In another embodiment, the alkyl group employed contains 1-8 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents.

The term alkoxy as used herein refers to a saturated (i.e., alkyl-O—) or unsaturated (i.e., alkenyl-O— and alkynyl-O—) group attached to the parent molecular moiety through an oxygen atom. In certain embodiments, the alkyl group contains 1-20 aliphatic carbon atoms. In certain other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples include, but are not limited to, methoxy, ethoxy, propoxy, isopropoxy, n-butoxy, tert-butoxy, i-butoxy, sec-butoxy, neopentoxy, n-hexoxy, and the like.

The term alkenyl denotes a monovalent group derived from a hydrocarbon moiety having at least one carbon-carbon double bond by the removal of a single hydrogen atom. In certain embodiments, the alkenyl group employed in the invention contains 1-20 carbon atoms. In some embodiments, the alkenyl group employed in the invention contains 1-10 carbon atoms. In another embodiment, the alkenyl group employed contains 1-8 carbon atoms. In still other embodiments, the alkenyl group contains 1-6 carbon atoms. In yet another embodiments, the alkenyl group contains 1-4 carbons. Alkenyl groups include, for example, ethenyl, propenyl, butenyl, 1-methyl-2-buten-1-yl, and the like.

The term alkynyl as used herein refers to a monovalent group derived form a hydrocarbon having at least one carbon-carbon triple bond by the removal of a single hydrogen atom. In certain embodiments, the alkynyl group employed in the invention contains 1-20 carbon atoms. In some embodiments, the alkynyl group employed in the invention contains 1-10 carbon atoms. In another embodiment, the alkynyl group employed contains 1-8 carbon atoms. In still other embodiments, the alkynyl group contains 1-6 carbon atoms. Representative alkynyl groups include, but are not limited to, ethynyl, 2-propynyl (propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. In certain embodiments, the alkyl group contain 1-20 aliphatic carbon atoms. In certain other embodiments, the alkyl group contains 1-10 aliphatic carbon atoms. In yet other embodiments, the alkyl group contains 1-8 aliphatic carbon atoms. In still other embodiments, the alkyl group contain 1-6 aliphatic carbon atoms. In yet other embodiments, the alkyl group contain 1-4 aliphatic carbon atoms. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of thioalkoxyl moieties include, but are not limited to, methylthio, ethylthio, propylthio, isopropylthio, n-butylthio, and the like.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms aryl and heteroaryl, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, aryl refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term heteroary, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term carboxylic acid as used herein refers to a group of formula —CO₂H.

The terms halo and halogen as used herein refer to an atom selected from fluorine, chlorine, bromine, and iodine.

The term haloalkyl denotes an alkyl group, as defined above, having one, two, or three halogen atoms attached thereto and is exemplified by such groups as chloromethyl, bromoethyl, trifluoromethyl, and the like.

The term heteroaliphatic, as used herein, refers to aliphatic moieties that contain one or more oxygen, sulfur, nitrogen, phosphorus, or silicon atoms, e.g. in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl, as used herein, refers to an amide group of the formula —CONH₂.

The term carbonyldioxyl, as used herein, refers to a carbonate group of the formula —O—CO—OR.

The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, isopropyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term thiohydroxyl or thiol, as used herein, refers to a group of the formula —SH.

The following are more general terms used throughout the present application:

As used herein, the singular forms “a”, “an”, and “the” include the plural reference unless the context clearly indicates otherwise. Thus, for example, a reference to “a monomer” includes a plurality of such monomers.

“Animal”: The term animal, as used herein, refers to humans as well as non-human animals, including, for example, mammals, birds, reptiles, amphibians, and fish. Preferably, the non-human animal is a mammal (e.g. a rodent, a mouse, a rat, a rabbit, a monkey, a dog, a cat, a primate, or a pig). An animal may be a domesticated animal. In certain embodiments, the animal is human. An animal may be a transgenic animal.

“Biocompatible”: The term “biocompatible”, as used herein is intended to describe compounds that are not toxic to cells. Compounds are “biocompatible” if their addition to cells in vitro results in less than or equal to 20% cell death. The administration of the compound in vivo does not cause cancer, birth defects, neurotoxicity, or other such adverse side effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably does not cause inflammation, cancer, birth defects, neurotoxicity, or other such adverse side effects in vivo. In certain preferred embodiments, the chemical reactions relied upon to break down the biodegradable compounds are uncatalyzed. For example, the inventive materials may be broken down in part by the hydrolysis of the ester bonds found in cross-linked material.

“Keratin”: The term “keratin” as used herein refers any one of a class of fibrous structural proteins found in skin. Keratin proteins contains a large quantity of cysteine residues. The helical keratin molecules twist around each other to form elongated strands called intermediate filaments.

“Monomer”: As used herein, a “monomer” is a chemical compound that is linked to other monomers covalently to form a polymer. Examples of monomers include acrylates, methacrylates, epoxide containing compounds, styrenes, and vinyl alcohol. In certain embodiments, the monomers useful in accordance with the present invention are susceptible to free radical polymerization.

“Oligomer”: The term “oligomer,” as used herein, refers to a chemical compound with a finite number of structural units connected by covalent bonds. An oligomer has less monomeric units than the corresponding polymer. An oligomer typically has between 3 to 100 monomeric units making up its structure. In certain embodiments, less than 10 monomeric units are found in the oligomer. In certain embodiments, less than 20 monomeric units are found in the oligomer. In certain embodiments, less than 50 monomeric units are found in the oligomer. In certain embodiments, less than 100 monomeric units are found in the oligomer.

“Peptide” or “protein”: As used herein, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polymer”: The term “polymer,” as used herein, refers to a chemical compound of repeating structural units (monomers) connected by covalent bonds. A polymer is typically of high molecular weight and may comprise 10s to 100s to 1000s or even more monomers. In certain embodiments, the polymer comprises at least 10 monomeric units linked covalently together. In certain embodiments, the polymer may be a co-polymer comprising different types of polymers. The polymer may be cross-linked or uncross-linked. The polymer may be linear or branched. In certain embodiments, the polymer is formed by in situ polymerization on skin.

### DETAILED DESCRIPTION OF CERTAIN EMBODIMENTS OF THE INVENTION

The present invention provides a system for the in situ polymerization of monomers (e.g., acrylates, methacrylates, dienes, maleimides, fluorinated monomers) on skin. The application of polymerizable monomers and a polymerization initiator to skin followed by initiation of polymerization leads to the formation of polymers on the surface of the treated skin. The polymerization of monomers on skin has been shown to change the appearance and/or feel of the treated skin. The inventive system can also be used to affect the color of the treated skin. The inventive system can also be used to protect the skin from harmful UV rays. The inventive treatment is robust and long-lasting resisting smudging, smearing, and flaking. The inventive treatment may also resist multiple washings.

One advantage of the present system is that certain polymers can not effectively be applied to skin via traditional means using pre-formed polymers given their low solubility. The polymeric film may also bond to the outer layer of skin providing a more long lasting cosmetic effect. In the inventive system, polymerizable monomers are applied to skin with a polymerization initiator, and the treated skin is then exposed to light or heat to cause the polymerization of the monomers in situ on the skin. Thus, the inventive system eliminates the need to formulate polymers with low solubility. Polymers that could not before be used on skin can now be prepared directly on the skin's surface. The polymers may be homopolymers with repeating units of the same type or heteropolymers with repeating units of two or more different types. In situ polymerization gives the user greater flexibility in treating skin. The invention provides methods, compositions, kits, and materials for treating skin using the inventive system.

### Polymerizable Monomers

A variety of polymerizable monomers may be used in accordance with the present invention to generate polymers in situ on skin. Some monomers generate polymers that are only available for skin treatment using the inventive in situ polymerization technique. Different monomers or combinations of monomers may be used to create polymers with different properties, thereby creating different cosmetic effects. The availability of a wide range of monomers for polymer generation also allows for the development of polymers with a wide variety of properties which include longevity, appearance, optical properties, feel, color, texture, etc.

A polymerizable monomer is any chemical compound (e.g. organic compound), regardless of molecular weight, that when exposed to a polymerization initiator reacts with other monomers to generate a polymer. In certain embodiments, the monomers are monomers in the strict sense of the term in that the monomer does not include a repeating unit. That is, the monomer is not an oligomer or low molecular weight polymer. In certain embodiments, the monomers are oligomers, resins, partially polymerized polymers, low molecular weight polymers, or uncross-linked polymers. In certain embodiments, the oligomers are of various molecular weights and may contain 2-50 monomer units. In certain embodiments, the oligomer contains 2-10 monomer units. In certain embodiments, the oligomer contains 2-20 monomer units.

In certain embodiments, the molecular weight of the monomer is less than about 2,000 g/mol. In certain other embodiments, the molecular weight of the monomer is less than about 1,500 g/mol. In certain other embodiments, the molecular weight of the monomer is less than about 1,000 g/mol. In certain embodiments, the molecular weight of the monomer is less than about 500 g/mol. In certain embodiments, the molecular weight of the monomer is less than about 400 g/mol. In certain embodiments, where monomer toxicity is an issue, monomers with higher molecular weights are preferred so as to decrease the ability of the monomer to pass through the skin. In certain embodiments, less than 5%, less than 2%, less than 1% or less than 0.1% of the applied monomer reaches the systemic circulation. In such embodiments, the molecular weight of the monomer is greater than 500 g/mol. In such embodiments, the molecular weight of the monomer is greater than 1,000 g/mol. In such embodiments, the molecular weight of the monomer is greater than 1,500 g/mol. In certain embodiments, the molecular weight of the monomer is greater than 2,000 g/mol. In certain embodiments, the molecular weight of the monomer is greater than 2,500 g/mol. In certain embodiments, the molecular weight of the monomer is greater than 3,500 g/mol. In certain embodiments, the molecular weight of the monomer is greater than 5,000 g/mol. In certain embodiments, the molecular weight of the monomer is greater than 10,000 g/mol.

The polymerizable monomer comprises a functional group suitable for polymerization. Any functional group that can be polymerized using a free radical or ionic polymerization reaction can be used. In certain embodiments, the monomers include a functional group with at least one degree of unsaturation. For example, the monomer includes a double bond or triple bond. Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc. In certain particular embodiments, the monomers comprise a vinyl group. In certain particular embodiments, the monomers comprise an acrylate functional group. In certain particular embodiments, the monomers comprise a methacrylate functional group. In certain particular embodiments, the monomers comprise a diene moiety. In certain embodiments, the monomers comprise a conjugated diene moiety. In certain embodiments, the monomers comprise a maleimide moiety. Other reactive functional groups may also be used including epoxides and halogen-containing compounds.

In certain embodiments, the monomer is an alkene. In certain particular embodiments, the alkene is monosubstituted. In other embodiments, the alkene is disubstituted. Disubstituted alkenes may be either in the cis or trans configuration or a mixture thereof. In yet other embodiments, the alkene is trisubstituted. The trisubstituted alkene may be in either the E or Z configuration or a mixture thereof. In still other embodiments, the alkene is tetrasubstituted. Again, various isomers are possible and are considered part of this invention. In certain embodiments, the monomer is an alkyne.

In certain embodiments, the monosubstituted monomer is of the formula:

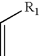

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁ is a substituted or unsubstituted, branched or unbranched aliphatic moiety. In certain embodiments, R₁ is an alkyl moiety. In certain embodiments, R₁ is of one of the formulae:

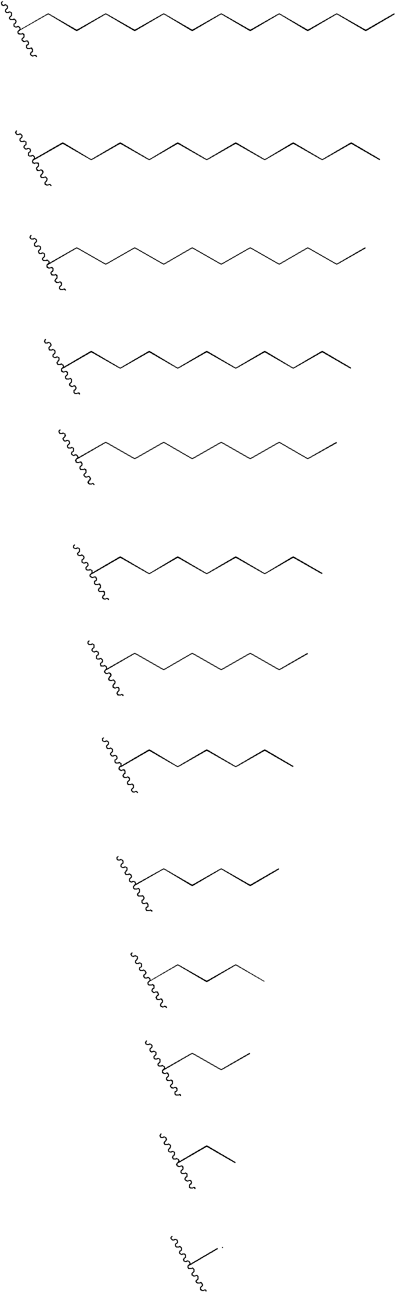

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

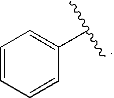

In certain particular embodiments, R₁ is of the formula:

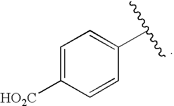

In certain particular embodiments, R₁ is a substituted or unsubstituted phenyl moiety. In certain embodiments, R₁ is substituted phenyl (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain particular embodiments, R₁ is —CO₂R_{A}, wherein R_{A} is one of the formulae:

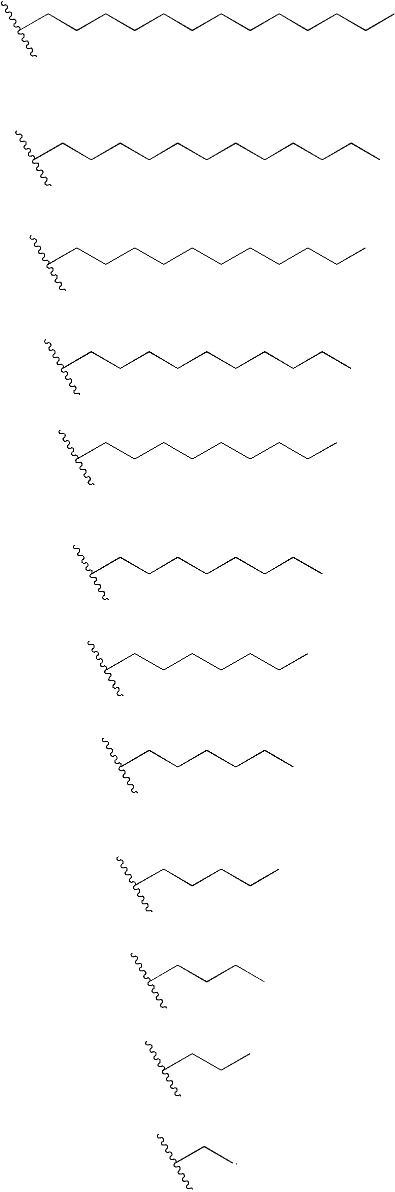

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

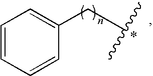

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

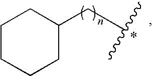

wherein n is an integer between 0 and 12, inclusive. In certain embodiments, n is 0. In certain embodiments, n is 1. In other embodiments, n is 2, 3, 4, 5, or 6.

In certain embodiments, the monomer is a acrylate of formula:

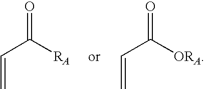

Exemplary acrylate monomers include:

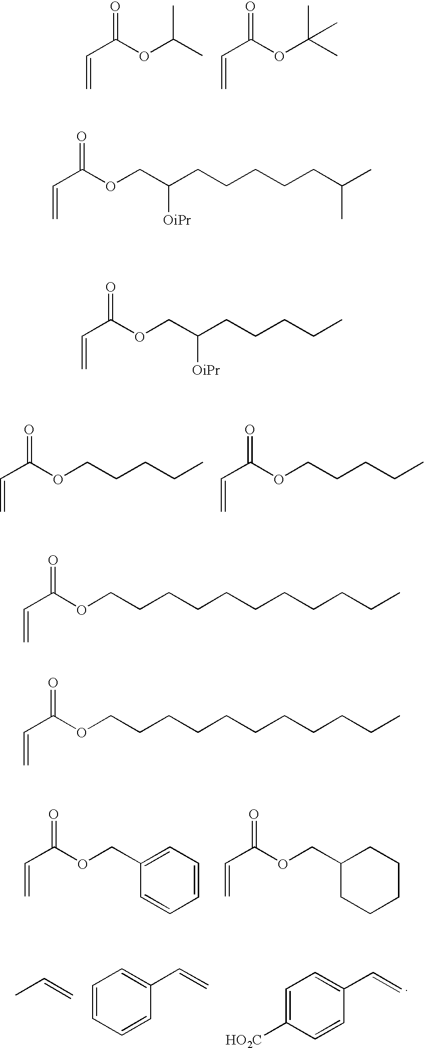

In certain embodiments, the disubstituted monomer is of one of the formulae:

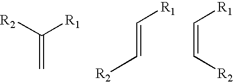

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁ and R₂ may form a cyclic structure, for example, a maleimide moiety.

In other embodiments, R₁ is a substituted or unsubstituted, branched or unbranched aliphatic moiety. In certain embodiments, R₁ is a alkyl moiety. In certain embodiments, R₁ is of one of the formulae:

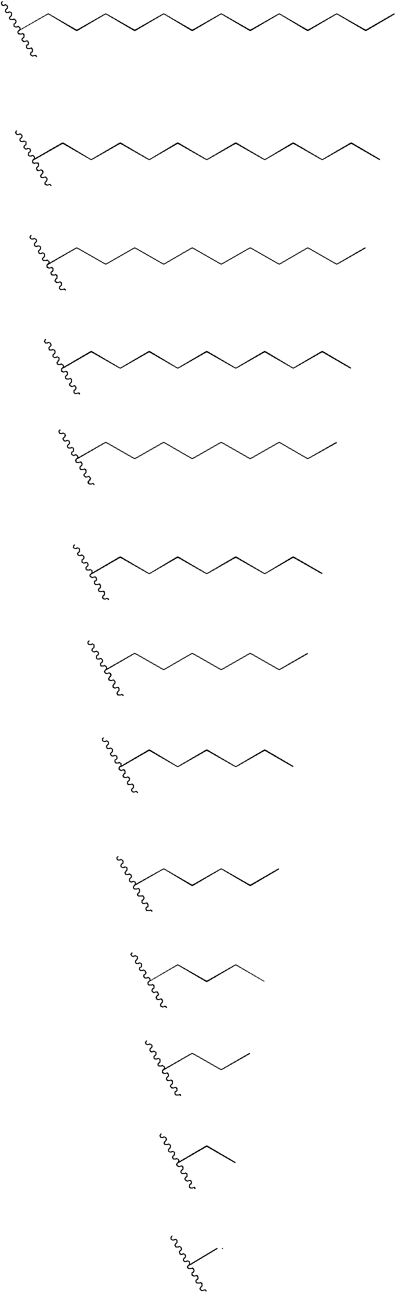

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

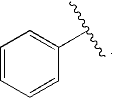

In certain particular embodiments, R₁ is of the formula:

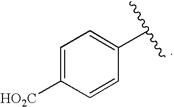

In certain particular embodiments, R₁ is a substituted or unsubstituted phenyl moiety. In certain embodiments, R₁ is substituted phenyl moiety (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

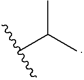

In other embodiments, R_{A} is t-butyl. In certain particular embodiments, R₁ is —CO₂R_{A}, wherein R_{A} is one of the formulae:

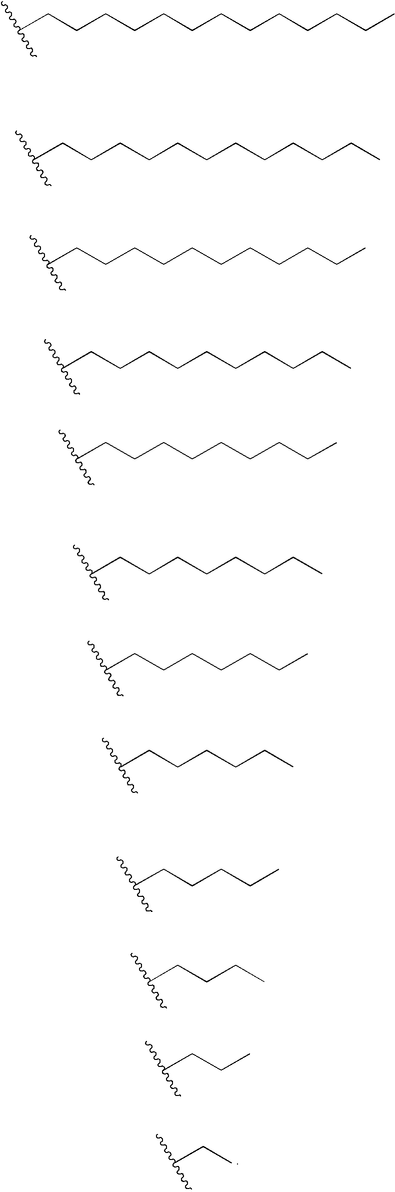

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

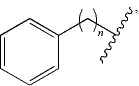

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

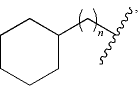

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain embodiments, R₂ is a alkyl moiety. In certain particular embodiments, R₂ is methyl. In certain embodiments, R₂ is a aryl or heteroaryl moiety. In certain embodiments, R₂ is a phenyl moiety. In certain particular embodiments, R₂ is a phenyl moiety.

In certain embodiments, R₁ is —CO₂R_{A}. In other embodiments, R₁ is —CO₂R_{A}, and R₂ is C₁-C₆ alkyl. In other embodiments, R₁ is —CO₂R_{A}, and R₂ is methyl.

In certain embodiments, the monomer is a methacrylate of formula:

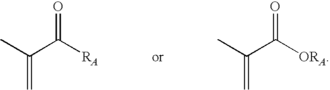

In certain embodiments, the monomer is a crotonate of formula:

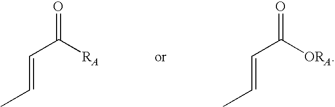

Exemplary disubstituted fluorinated monomers include:

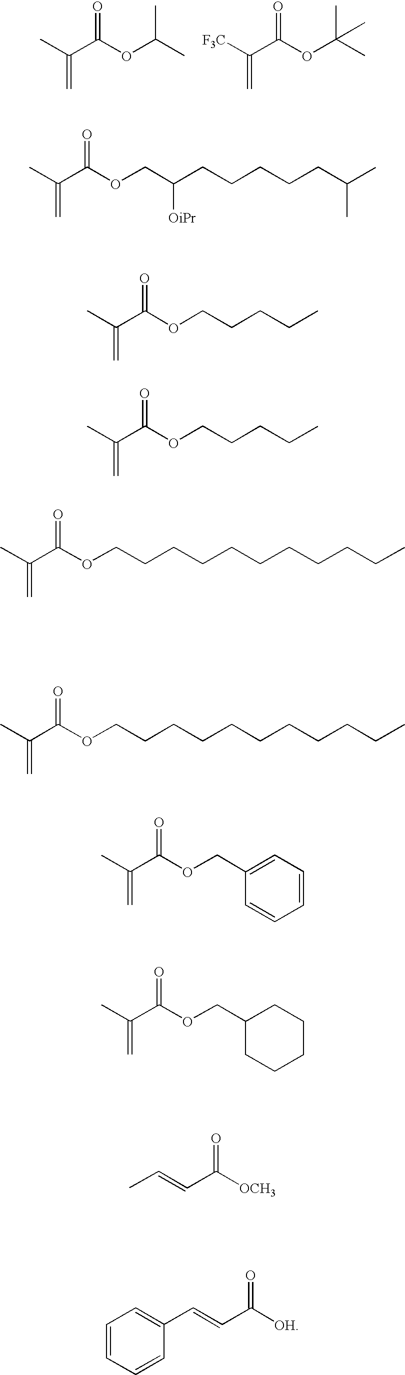

In certain embodiments, the trisubstituted fluorinated monomer is of one of the formulae:

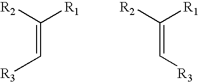

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁, R₂, and/or R₃ may form a cyclic structure.

In other embodiments, R₁ is a substituted or unsubstituted, branched or unbranched aliphatic moiety. In certain embodiments, R₁ is a alkyl moiety. In certain embodiments, R₁ is of one of the formulae:

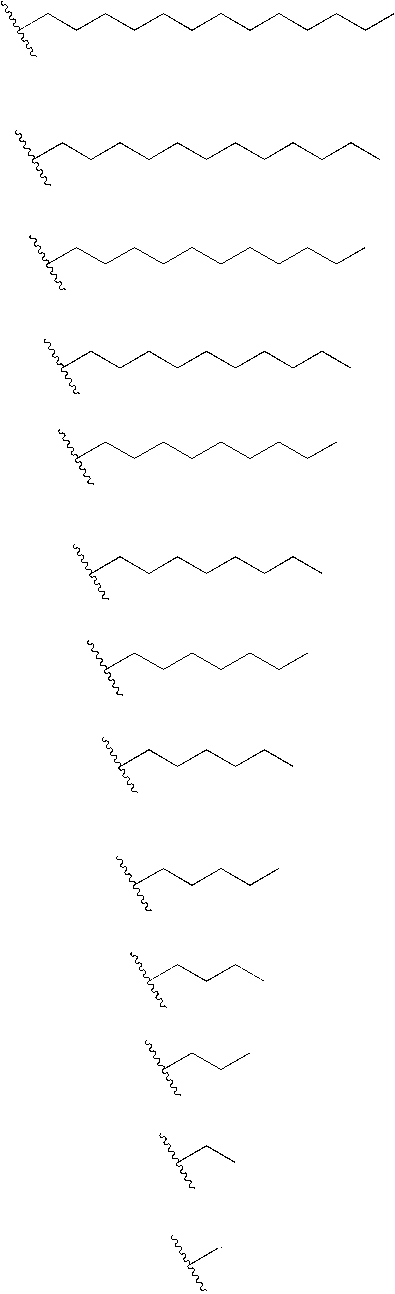

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

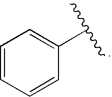

In certain particular embodiments, R₁ is of the formula:

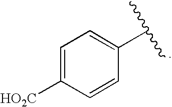

In certain particular embodiments, R₁ is a substituted or unsubstituted phenyl moiety. In certain embodiments, R₁ is substituted phenyl moiety (e.g., a phenyl ring with 1, 2, 3, 4, or 5 substituents). In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

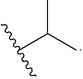

In other embodiments, R_{A} is t-butyl. In certain particular embodiments, R₁ is —CO₂R_{A}, wherein R_{A} is one of the formulae:

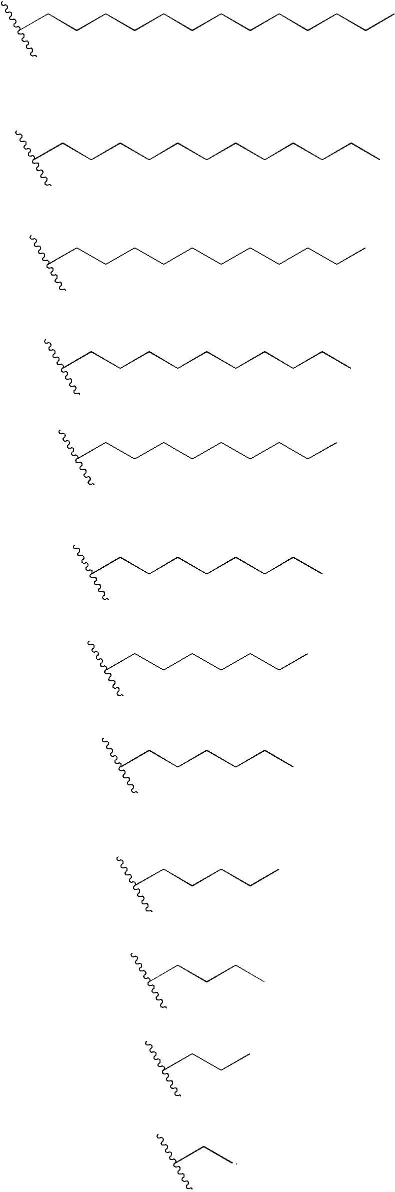

As would be appreciated by one of skill in this art, any of the above alkyl group may be partially substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

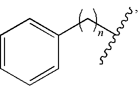

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

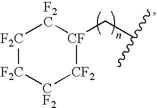

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain particular embodiments, R₂ is methyl.

In certain embodiments, R₂ is of one of the formulae:

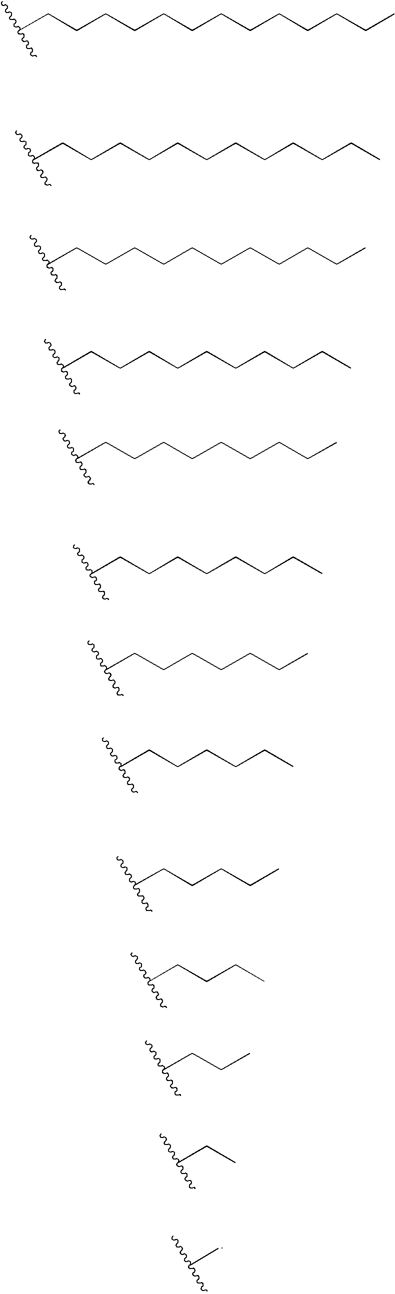

As would be appreciated by one of skill in this art, any of the above alkyl group may be substituted, branched, unsaturated, and/or cyclic. In yet other embodiments, R₂ is a substituted or unsubstituted, branched or unbranched heteroaliphatic moiety. In still other embodiments, R₂ is a substituted or unsubstituted acyl moiety. In other embodiments, R₂ is a substituted or unsubstituted aryl moiety. In certain particular embodiments, R₂ is of the formula:

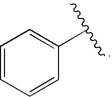

In certain particular embodiments, R₂ is of the formula:

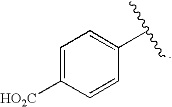

In certain particular embodiments, R₂ is a substituted or unsubstituted phenyl moiety. In certain embodiments, R₂ is substituted phenyl moiety (e.g., a phenyl ring with 1, 2, 3, 4, or 5 substituents). In other embodiments, R₂ is a substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain embodiments, R_{B} is

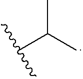

In other embodiments, R_{B} is t-butyl. In certain particular embodiments, R₂ is —CO₂R_{B}, wherein R_{B} is one of the formulae:

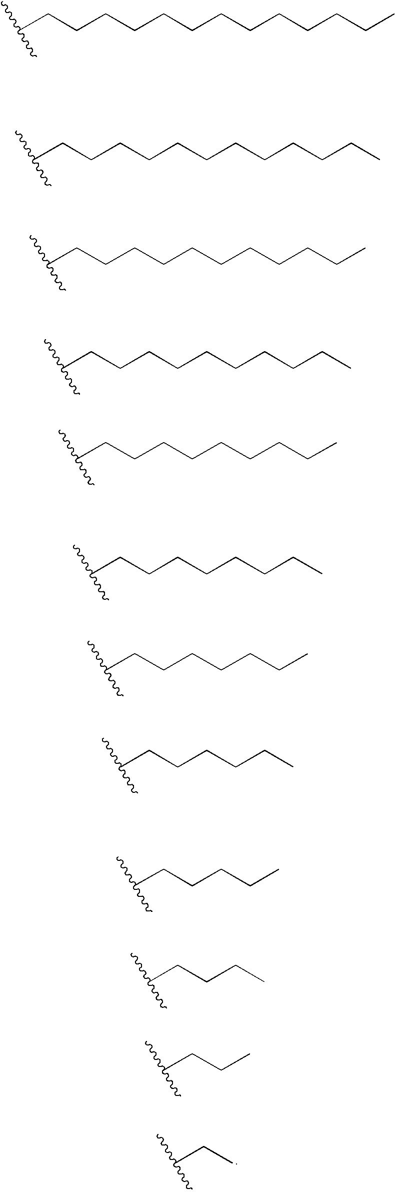

As would be appreciated by one of skill in this art, any of the above alkyl groups may be substituted, branched, unsaturated, and/or cyclic. In certain particular embodiments, R₂ is —CO₂R_{B}, wherein R_{B} is aryl or arylalkyl. In certain particular embodiments, R₂ is —CO₂R_{B}, wherein R_{B} is of the formula:

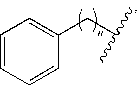

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

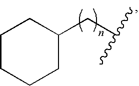

wherein n is an integer between 0 and 12, inclusive. In certain embodiments, n is 0. In certain embodiments, n is 1. In other embodiments, n is 2, 3, 4, 5, or 6.

In other embodiments, R₃ is substituted or unsubstituted, branched or unbranched aliphatic. In yet other embodiments, R₃ is C₁-C₆ alkyl. In certain particular embodiments, R₃ is methyl.

In certain embodiments, R₃ is of one of the formulae:

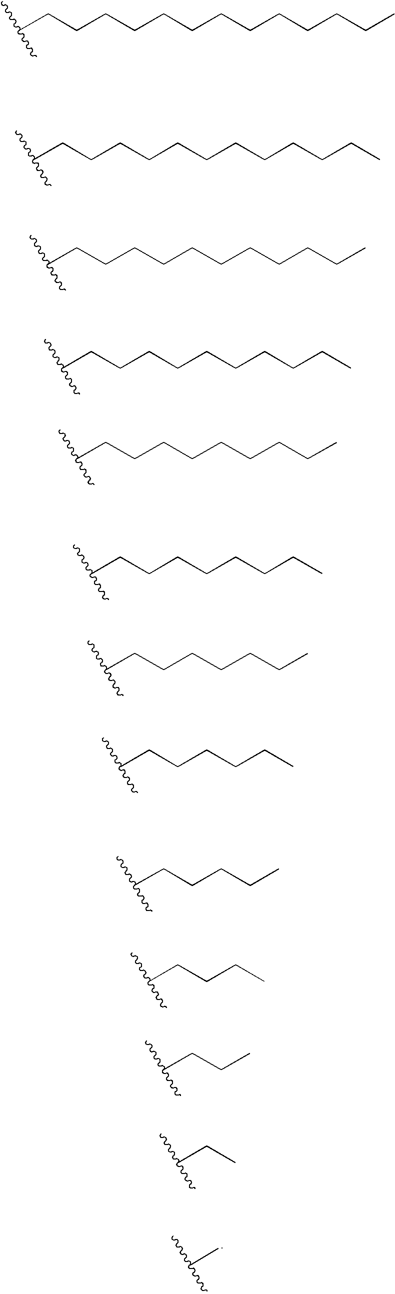

As would be appreciated by one of skill in this art, any of the above alkyl groups may be substituted, branched, unsaturated, and/or cyclic. In yet other embodiments, R₃ is a substituted or unsubstituted, branched or unbranched heteroaliphatic moiety. In still other embodiments, R₃ is a substituted or unsubstituted acyl moiety. In other embodiments, R₃ is a substituted or unsubstituted aryl moiety. In certain particular embodiments, R₃ is of the formula:

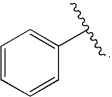

In certain particular embodiments, R₃ is of the formula:

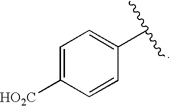

In certain particular embodiments, R₃ is a substituted or unsubstituted phenyl moiety. In certain embodiments, R₃ is a substituted phenyl moiety (e.g., a phenyl ring with 1, 2, 3, 4, or substituents). In other embodiments, R₃ is a substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain embodiments, R_{C} is

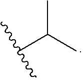

In other embodiments, R_{C} is t-butyl. In certain particular embodiments, R₃ is —CO₂R_{A}, wherein R_{C} is one of the formulae:

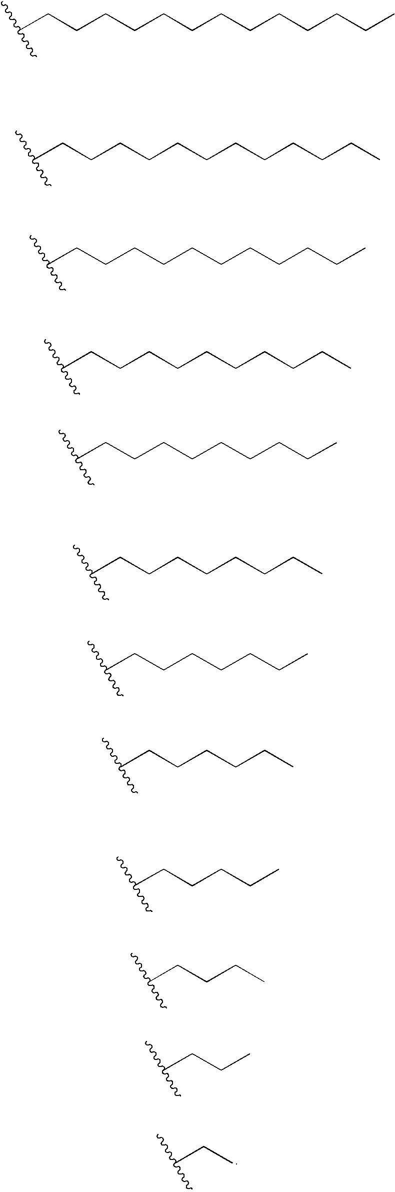

As would be appreciated by one of skill in this art, any of the above alkyl groups may be substituted, branched, unsaturated, and/or cyclic. In certain particular embodiments, R₃ is —CO₂R_{C}, wherein R_{C} is aryl or arylalkyl. In certain particular embodiments, R₃ is —CO₂R_{C}, wherein R_{C} is of the formula:

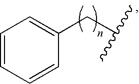

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

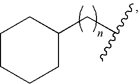

wherein n is an integer between 0 and 12, inclusive. In certain embodiments, n is 0. In certain embodiments, n is 1. In other embodiments, n is 2, 3, 4, 5, or 6.

In other embodiments, R₁ is —CO₂R_{A}, and R₂ and R₃ are both methyl.

Exemplary trisubstituted fluorinated monomers include:

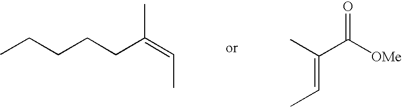

In certain embodiments, the tetrasubstituted fluorinated monomer is of one of the formulae:

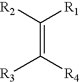

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —C(═O)N(R_{D})₂; —CN; —SCN; —SR_{D}; —SOR_{D}; —SO₂R_{D}; —NO_{D}; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. R₁, R₂, R₃, and/or R₄ may form a cyclic structure.

In other embodiments, R₁ is a substituted or unsubstituted, branched or unbranched aliphatic moiety. In certain embodiments, R₁ is of one of the formulae:

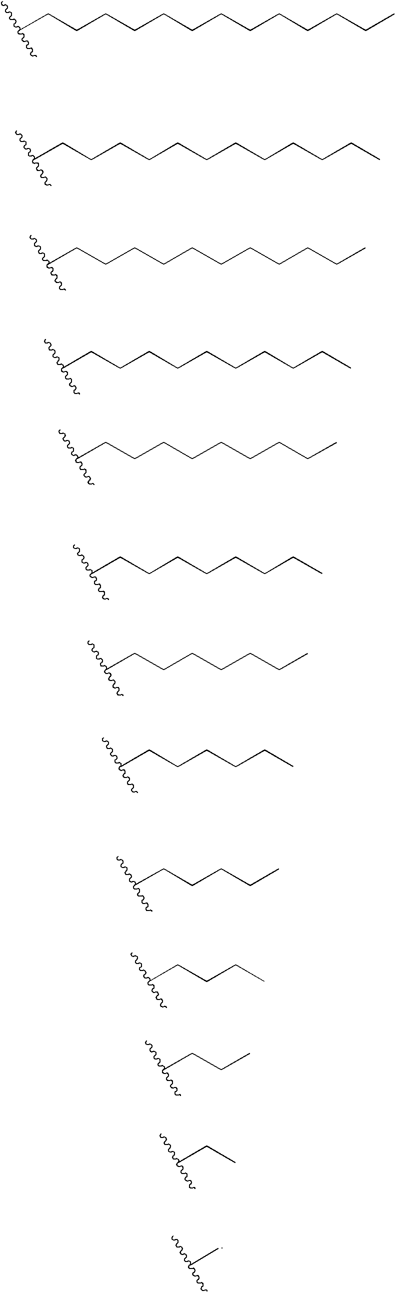

As would be appreciated by one of skill in this art, any of the above alkyl groups may be partially substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

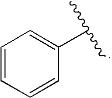

In certain particular embodiments, R₁ is of the formula:

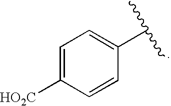

In certain particular embodiments, R₁ is a substituted or unsubstituted phenyl moiety. In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

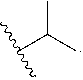

In other embodiments, R_{A} is t-butyl. In certain particular embodiments, R₁ is —CO₂R_{A}, wherein R_{A} is one of the formulae:

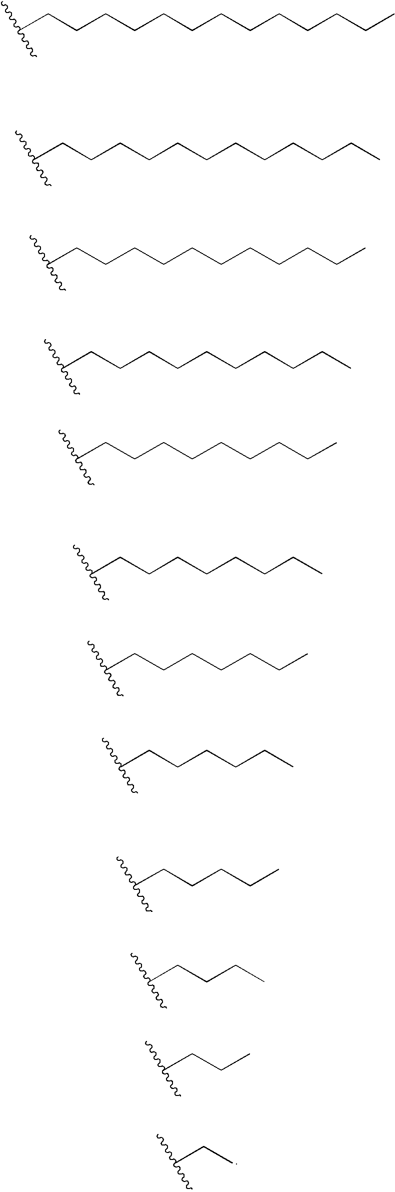

As would be appreciated by one of skill in this art, any of the above alkyl groups may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

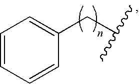

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

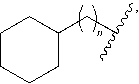

wherein n is an integer between 0 and 12, inclusive. In certain embodiments, n is 0. In certain embodiments, n is 1. In other embodiments, n is 2, 3, 4, 5, or 6.

In other embodiments, R₂ is substituted or unsubstituted, branched or unbranched aliphatic. In yet other embodiments, R₂ is C₁-C₆ alkyl. In certain particular embodiments, R₂ is methyl.

In certain embodiments, R₂ is of one of the formulae:

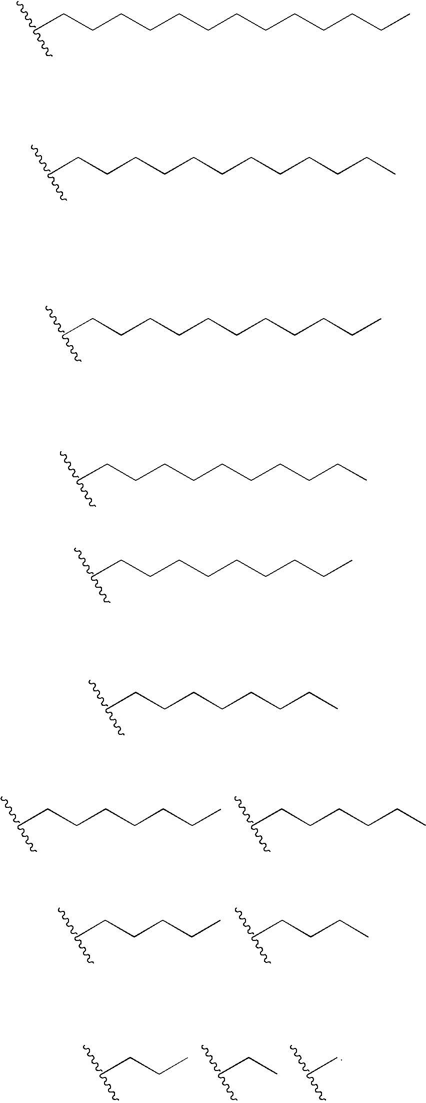

As would be appreciated by one of skill in this art, any of the above alkyl groups may be substituted, branched, unsaturated, and/or cyclic. In yet other embodiments, R₂ is a substituted or unsubstituted, branched or unbranched heteroaliphatic moiety. In still other embodiments, R₂ is a substituted or unsubstituted acyl moiety. In other embodiments, R₂ is a substituted or unsubstituted aryl moiety. In certain particular embodiments, R₂ is of the formula:

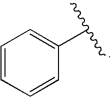

In certain particular embodiments, R₂ is of the formula:

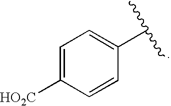

In certain particular embodiments, R₂ is a substituted or unsubstituted phenyl moiety. In certain embodiments, R₂ is substituted phenyl moiety (e.g., a phenyl ring with 1, 2, 3, 4, or 5 substituents). In other embodiments, R₂ is a substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain embodiments, R_{B} is

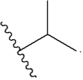

In other embodiments, R_{B} is t-butyl. In certain particular embodiments, R₂ is —CO₂R_{B}, wherein R_{B} is one of the formulae:

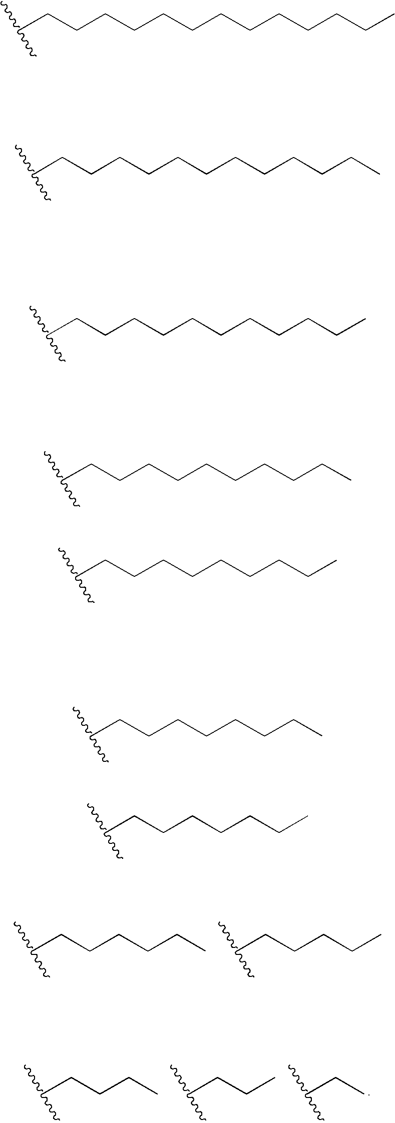

As would be appreciated by one of skill in this art, any of the above alkyl groups may be substituted, branched, unsaturated, and/or cyclic. In certain particular embodiments, R₂ is —CO₂R_{B}, wherein R_{B} is aryl or arylalkyl. In certain particular embodiments, R₂ is —CO₂R_{B}, wherein R_{B} is of the formula:

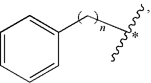

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

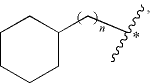

wherein n is an integer between 0 and 12, inclusive. In certain embodiments, n is 0. In certain embodiments, n is 1. In other embodiments, n is 2, 3, 4, 5, or 6.

In other embodiments, R₃ is substituted or unsubstituted, branched or unbranched aliphatic. In yet other embodiments, R₃ is C₁-C₆ alkyl. In certain particular embodiments, R₃ is methyl.

In certain embodiments, R₃ is of one of the formulae:

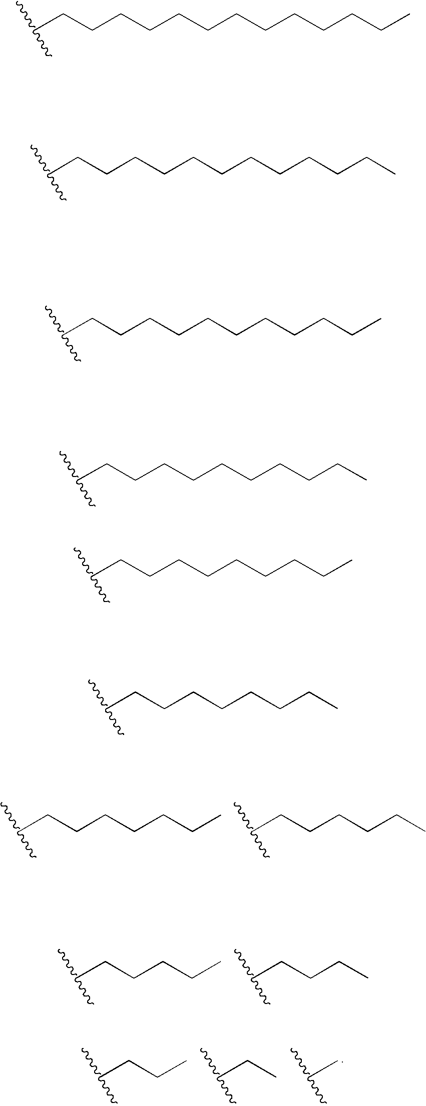

As would be appreciated by one of skill in this art, any of the above alkyl groups may be substituted, branched, unsaturated, and/or cyclic. In yet other embodiments, R₃ is a substituted or unsubstituted, branched or unbranched heteroaliphatic moiety. In still other embodiments, R₃ is a substituted or unsubstituted acyl moiety. In other embodiments, R₃ is a substituted or unsubstituted aryl moiety. In certain particular embodiments, R₃ is of the formula:

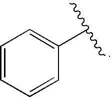

In certain particular embodiments, R₃ is of the formula:

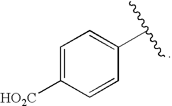

In certain particular embodiments, R₃ is a substituted or unsubstituted phenyl moiety. In certain embodiments, R₃ is a substituted phenyl (e.g., a phenyl ring with 1, 2, 3, 4, or 5 substituents). In other embodiments, R₃ is a substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain embodiments, R_{C} is

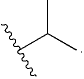

In other embodiments, R_{C} is t-butyl. In certain particular embodiments, R₃ is —CO₂R_{C}, wherein R_{C} is one of the formulae:

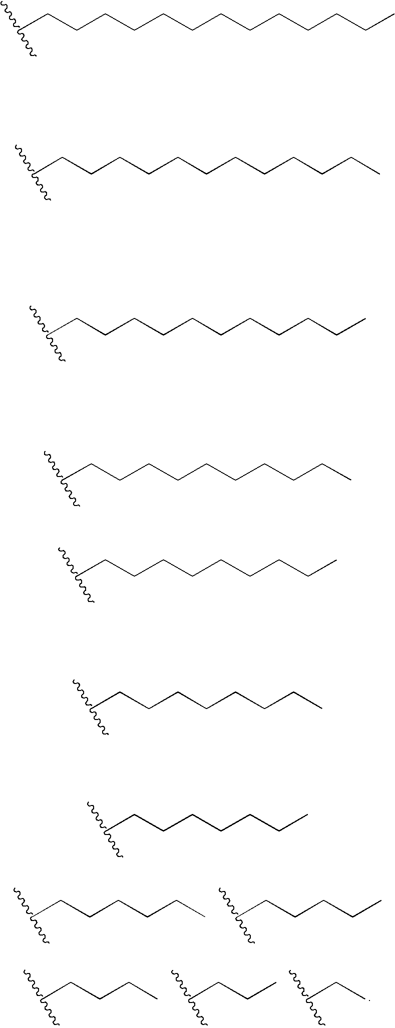

As would be appreciated by one of skill in this art, any of the above alkyl groups may be substituted, branched, unsaturated, and/or cyclic. In certain particular embodiments, R₃ is —CO₂R_{C}, wherein R_{C} is aryl or arylalkyl. In certain particular embodiments, R₃ is —CO₂R_{C}, wherein R_{C} is of the formula:

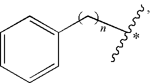

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

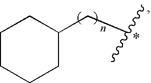

wherein n is an integer between 0 and 12, inclusive. In certain embodiments, n is 0. In certain embodiments, n is 1. In other embodiments, n is 2, 3, 4, 5, or 6.

In other embodiments, R₄ is substituted or unsubstituted, branched or unbranched aliphatic. In yet other embodiments, R₄ is C₁-C₆ alkyl. In certain particular embodiments, R₄ is methyl.

In certain embodiments, R₄ is of one of the formulae:

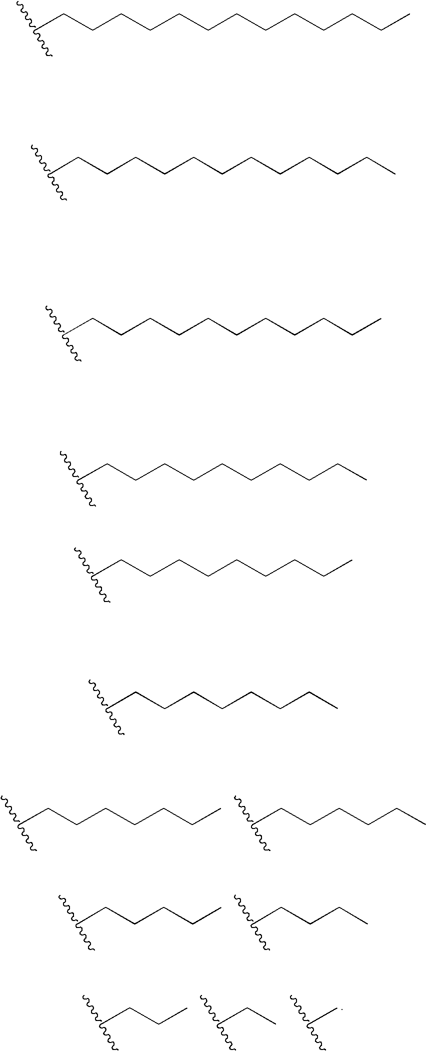

As would be appreciated by one of skill in this art, any of the above alkyl groups may be substituted, branched, unsaturated, and/or cyclic. In yet other embodiments, R₄ is a substituted or unsubstituted, branched or unbranched heteroaliphatic moiety. In still other embodiments, R₄ is a substituted or unsubstituted acyl moiety. In other embodiments, R₄ is a substituted or unsubstituted aryl moiety. In certain particular embodiments, R₄ is of the formula:

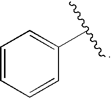

In certain particular embodiments, R₄ is of the formula:

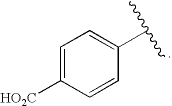

In certain particular embodiments, R₄ is a substituted or unsubstituted phenyl moiety. In certain embodiments, R₄ is substituted phenyl moiety (e.g., a phenyl ring with 1, 2, 3, 4, or 5 substituents). In other embodiments, R₄ is a substituted or unsubstituted heteroaryl moiety. In certain embodiments, R₄ is —C(═O)R_{D}. In other embodiments, R₄ is —CO₂R_{D}. In certain embodiments, R_{D} is C₁-C₆ alkyl. In certain particular embodiments, R_{D} is methyl. In certain embodiments, R_{D} is

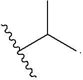

In other embodiments, R_{D} is t-butyl. In certain particular embodiments, R₄ is —CO₂R_{D}, wherein R_{D} is one of the formulae:

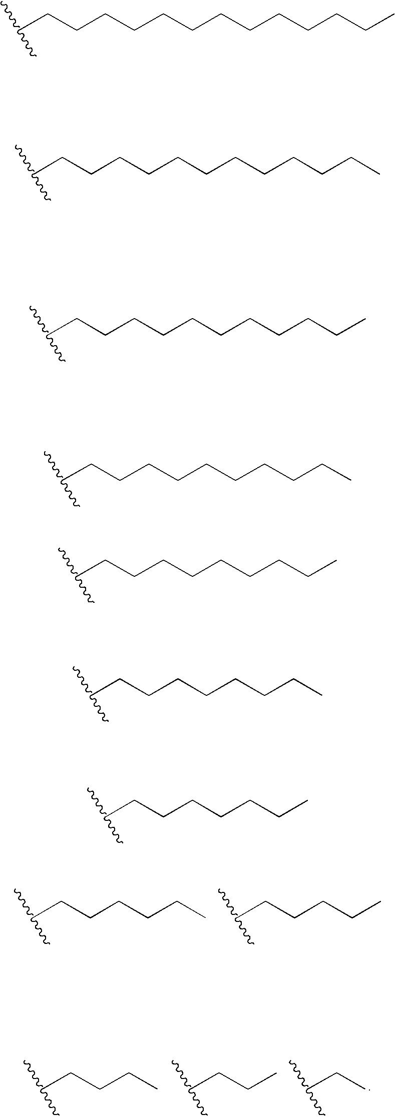

As would be appreciated by one of skill in this art, any of the above alkyl group may be substituted, branched, unsaturated, and/or cyclic. In certain particular embodiments, R₄ is —CO₂R_{D}, wherein R_{D} is aryl or arylalkyl. In certain particular embodiments, R₄ is —CO₂R_{D}, wherein R_{D} is of the formula:

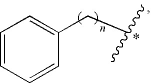

wherein n is an integer between 0 and 12, inclusive. In certain embodiments, n is 0. In certain embodiments, n is 1. In other embodiments, n is 2, 3, 4, 5, or 6. In certain particular embodiments, R₄ is —CO₂R_{D}, wherein R_{D} is of the formula:

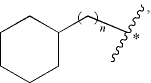

wherein n is an integer between 0 and 12, inclusive. In certain embodiments, n is 0. In certain embodiments, n is 1. In other embodiments, n is 2, 3, 4, 5, or 6.

In other embodiments, R₁ is —CO₂R_{A}, and R₂ and R₃ are both methyl. In certain embodiments, at least one of R₁, R₂, R₃, and R₄ is fluorine.

Exemplary tetrasubstituted fluorinated monomers include:

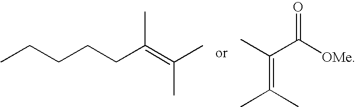

In certain embodiments, the monomer is a diacrylate or dimethacrylate. In certain embodiments, the fluorinated diacrylate is of the formula:

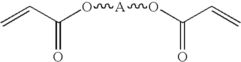

wherein A is a linker. In certain embodiments, the diacrylate is of the formula:

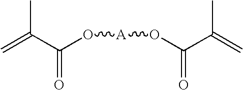

wherein A is a linker. In certain embodiments, A is a substituted or unsubstituted, branched or unbranched, cyclic or acyclic aliphatic; substituted or unsubstituted, branched or unbranched, cyclic or acyclic heteroaliphatic; substituted or unsubstituted aryl; or substituted or unsubstituted heteroaryl. In certain embodiments, the linker A is an alkyl linker. In certain embodiments, the linker A is of one of the formulae:

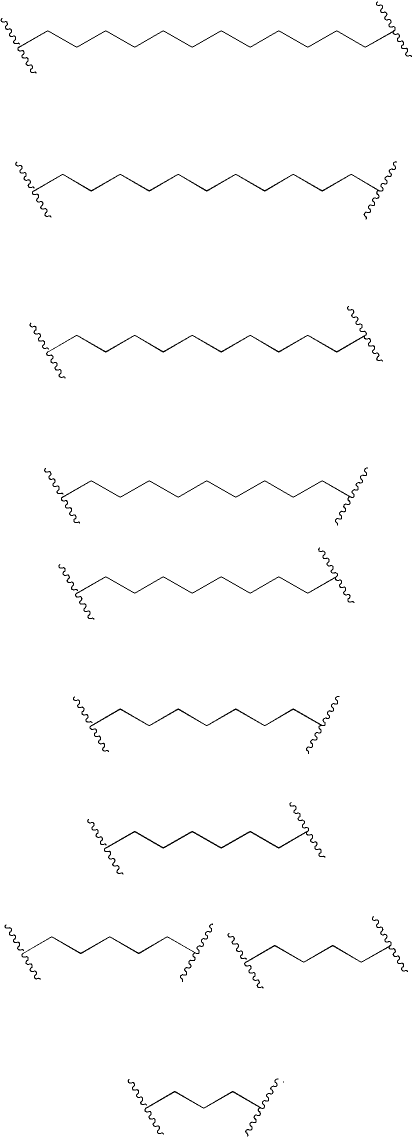

As would be appreciated by one of skill in this art, any of the above alkyl groups may be substituted, branched, unsaturated, and/or cyclic. In other embodiments, the linker A is of one of the formulae:

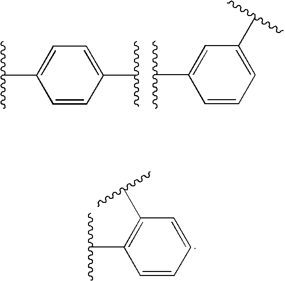

Exemplary diacrylate and dimethacrylates include:

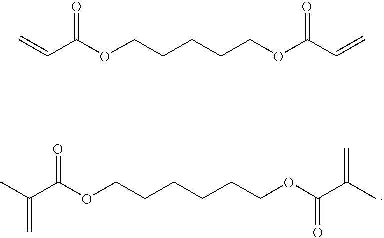

In certain embodiments, the monomer is a triacrylate or trimethacrylate. In certain embodiments, the monomer is of the formula:

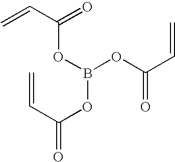

wherein B is a linker.

In other embodiments, the monomer is of the formula:

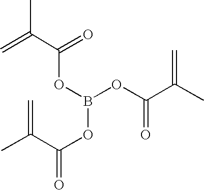

wherein B is a linker.

In certain embodiments, linker B is a substituted or unsubstituted, branched or unbranched, cyclic or acyclic aliphatic; substituted or unsubstituted, branched or unbranched, cyclic or acyclic heteroaliphatic; substituted or unsubstituted aryl; or substituted or unsubstituted heteroaryl. In certain embodiments, the linker B is a branched, alkyl linker. In certain embodiments, the linker B is a aryl linker. In certain embodiments, the linker B is of the formula:

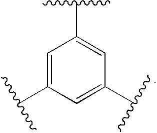

An exemplary trimethacrylate is of the formula:

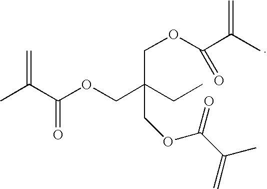

In certain embodiments, the fluorinated monomer is a tetraacrylate or tetramethacrylate. Tetraacrylates may be prepared by reacting diacrylates or dimethacrylates with a diamine. An exemplary tetramethacrylate is of the formula:

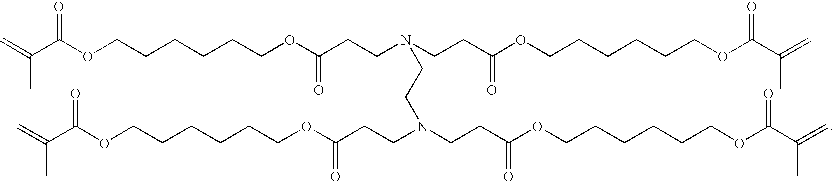

In other embodiments, the monomer is a pentaacrylate or pentamethacrylate. In still other embodiments, the monomer is an even higher acrylate or methacrylate.

In certain embodiments, the monomer is an alkyne. In certain embodiments, the alkynyl monomer is of the formula:

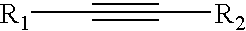

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁ is hydrogen. In other embodiments, R₁ is a substituted or unsubstituted, branched or unbranched aliphatic moiety. In certain embodiments, R₁ is of one of the formulae:

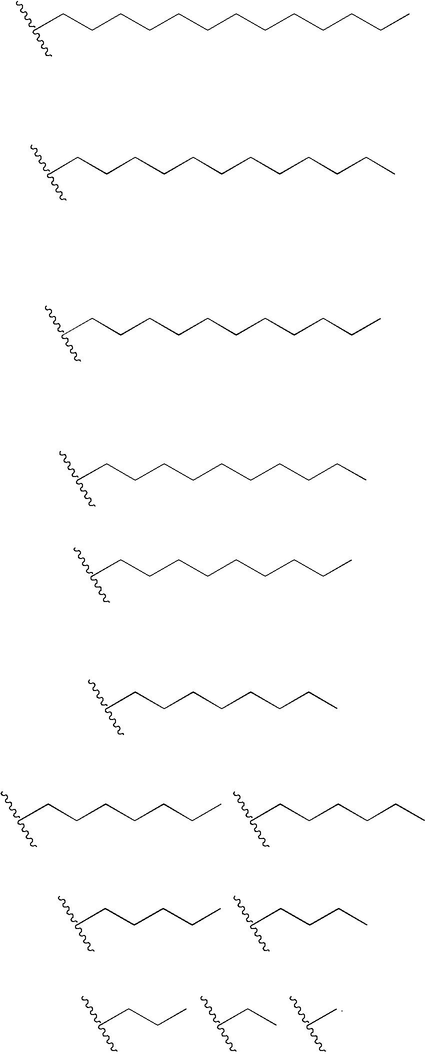

As would be appreciated by one of skill in this art, any of the above alkyl groups may be substituted, branched, unsaturated, and/or cyclic. In yet other embodiments, R₁ is a substituted or unsubstituted, branched or unbranched heteroaliphatic moiety. In still other embodiments, R₁ is a substituted or unsubstituted acyl moiety. In other embodiments, R₁ is a substituted or unsubstituted aryl moiety. In certain particular embodiments, R₁ is of the formula:

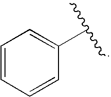

In certain particular embodiments, R₁ is of the formula:

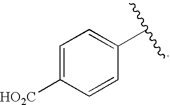

In certain particular embodiments, R₁ is a substituted or unsubstituted phenyl moiety. In other embodiments, R₁ is a substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain embodiments, R_{A} is

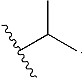

In other embodiments, R_{A} is t-butyl. In certain particular embodiments, R₁ is —CO₂R_{A}, wherein R_{A} is one of the formulae:

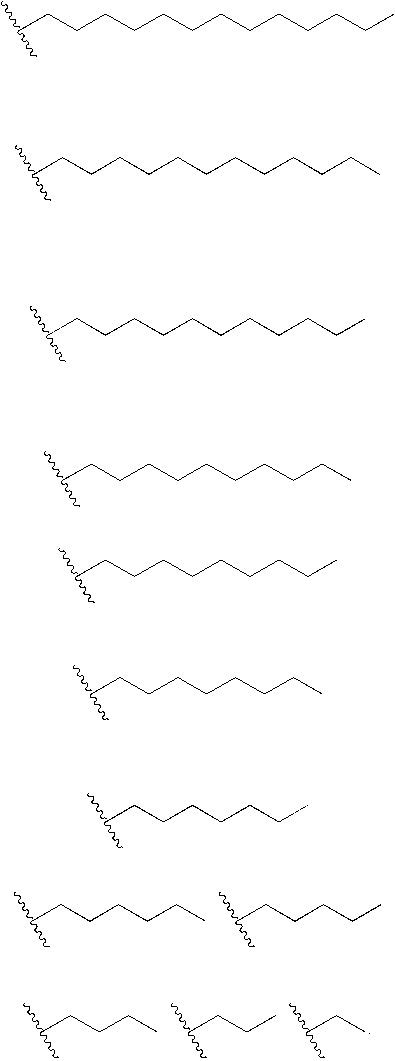

As would be appreciated by one of skill in this art, any of the above alkyl group may be substituted, branched, unsaturated, and/or cyclic. In certain particular embodiments, R₁ is —CO₂R_{A}, wherein R_{A} is aryl or arylalkyl. In certain particular embodiments, R₁ is —CO₂R_{A}, wherein R_{A} is of the formula:

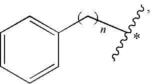

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

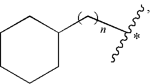

wherein n is an integer between 0 and 12, inclusive. In certain embodiments, n is 0. In certain embodiments, n is 1. In other embodiments, n is 2, 3, 4, 5, or 6.

In other embodiments, R₂ is hydrogen. In other embodiments, R₂ is substituted or unsubstituted, branched or unbranched aliphatic. In certain embodiments, R₂ is an alkyl moiety. In yet other embodiments, R₂ is C₁-C₆ alkyl. In certain particular embodiments, R₂ is methyl. In certain embodiments, R₂ is a aryl or heteroaryl moiety. In certain embodiments, R₂ is a phenyl moiety.

In certain embodiments, R₁ is —CO₂R_{A}. In certain embodiments, R₁ is —CO₂R_{A}, and R₂ is hydrogen. In other embodiments, R₁ is —CO₂R_{A}, and R₂ is methyl.

Exemplary fluorinated alkynyl monomers include:

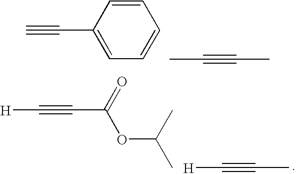

In certain other embodiments, the monomer is an oligomer. The monomers described herein are partially polymerized to form oligomers. The oligomers are applied to skin and further polymerized on the treated skin. In certain embodiments, the oligomers are of a molecular weight sufficient to apply the oligomer to skin. In certain embodiments, the molecular weight of the oligomer is less than 1,000 g/mol. In certain embodiments, the molecular weight is less than 1,500 g/mol. In other embodiments, the molecular weight is less than 2,000 g/mol. In other embodiments, the molecular weight is less than 3,000 g/mol. In other embodiments, the molecular weight is less than 4,000 g/mol. In yet other embodiments, the molecular weight is less than 5,000 g/mol.

In certain embodiments, the monomer is mixed with one or more different monomers. The resulting polymer is a co-polymer. As would be appreciated by those of skill in this art, a co-polymer may have desirable properties not attainable with a polymer resulting from the polymerization of one monomer alone. In certain embodiments, two different monomers are applied to skin. In other embodiments, three different monomers are applied to skin. When different monomer are used, the monomers are applied to skin simultaneously or separately. In certain embodiments, the monomers are all in the same solution which is applied to the skin.

Exemplary monomers useful in accordance with the present invention include trimethylolpropane trimethacrylate; 1,3-bis(3-methacryloyloxypropyl)-1,1,3,3-tetramethyldisiloxane; 1,3-butanediol dimethacrylate; 1,4-butanediol dimethacrylate; 1,6-hexanediol dimethacrylate; bisphenol A dimethacrylate; bisphenol A ethoxylate dimethacrylate; bisphenol A glycerolate dimethacrylate; di(ethylene glycol) dimethacrylate; diurethane dimethacrylate, mixture of isomers; ethylene glycol dimethacrylate; glycerol dimethacrylate, mixture of isomers; neopentyl glycol dimethacrylate; poly(ethylene glycol) dimethacrylate; poly(lauryl methacrylate-co-ethylene glycol dimethacrylate); poly(methyl methacrylate-co-ethylene glycol dimethacrylate); poly(propylene glycol) dimethacrylate; tetraethylene glycol dimethacrylate; triethylene glycol dimethacrylate; 1,1,1,3,3,3-hexafluoroisopropyl methacrylate; 2-(9H-carbazol-9-yl)ethyl acrylate; 2-(diethylamino)ethyl methacrylate; 2-(dimethylamino)ethyl methacrylate; 2-(methacryloyloxy)ethyl acetoacetate; 2-(methylthio)ethyl methacrylate; 2-(tert-butylamino)ethyl methacrylate; 2-(trimethylsilyloxy)ethyl methacrylate; 2,2,2-trifluoroethyl methacrylate; 2,2,3,3,3-pentafluoropropyl methacrylate; 2,2,3,3,4,4,4-heptafluorobutyl methacrylate; 2,2,3,3,4,4,5,5-octafluoropentyl methacrylate; 2,2,3,4,4,4-hexafluorobutyl methacrylate; 2-[3-(2H-benzotriazol-2-yl)-4-hydroxyphenyl]ethyl methacrylate; 2-aminoethyl methacrylate hydrochloride; 2-butoxyethyl methacrylate; 2-ethoxyethyl methacrylate; 2-ethylhexyl methacrylate; 2-hydroxyethyl methacrylate; 2-methyl-2-nitropropyl methacrylate; 2-naphthyl methacrylate; 3-(acryloyloxy)-2-hydroxypropyl methacrylate; 3-(diethoxymethylsilyl)propyl methacrylate; 3-(dimethylchlorosilyl)propyl methacrylate; 3-(trichlorosilyl)propyl methacrylate; 3-(dimethylchlorosilyl)propyl methacrylate; 3-(trichlorosilyl)propyl methacrylate; 3-(trimethoxysilyl)propyl methacrylate; 3,3,4,4,5,5,6,6,6,-nonafluorohexyl methacrylate; 3,3,4,4,5,5,6,6,7,7,8,8,8-tridecafluorooctyl methacrylate; 3,3,4,4,5,5,6,6,7,7,8,8,9,10,10,10-hexadecafluoro-9-trifluoromethyl)decyl methacrylate; 3,3,4,4,5,5,6,6,7,7,8,8,9,9,10,10,10-heptadecafluorodecyl methacrylate; 3,3,4,4,5,5,6,6,7,7,8,8,9,9,10,10,11,11,12,12,12-heneicosafluorododecyl methacrylate; 3,3,4,4,5,5,6,6,7,7,8,8,9,9,10,10,11,12,12,12-eicosafluoro-11-(trifluoromethyl)dodecyl methacrylate; 3,3,4,4,5,5,6,6,7,8,8,8-dodecafluoro-7-(trifluoromethyl)octyl methacrylate; 3,3,4,4,5,6,6,6-octafluoro-5-(trifluoromethyl)hexyl methacrylate; 3,3,5-trimethylcyclohexyl methacrylate, mixture of isomers; 3-[(3,5,7,9,11,13,15-heptacyclopentylpentacyclo[9.5.1.1^{3,9}.1^{5,15}.1^{7,13}]octasiloxan-1-yloxy)dimethylsilyl]propyl methacrylate; 3-[tris(trimethylsiloxy)silyl]propyl methacrylate; 3-chloro-2-hydroxypropyl methacrylate; 3-sulfopropyl methacrylate; 4,4,5,5,6,6,7,7,8,8,9,9,10,11,11,11-hexadecafluoro-2-hydroxy-10-(trifluoromethyl)undecyl methacrylate; 4,4,5,5,6,6,7,7,8,9,9,9-dodecafluoro-2-hydroxy-8-(trifluoromethyl)nonyl methacrylate; 4,4,5,5,6,7,7,7-octafluoro-2-hydroxy-6-(trifluoromethyl)heptyl methacrylate; 6-[4-(4-cyanophenyl)phenoxy]hexyl methacrylate; 9-anthracenylmethyl methacrylate; 9H-carbazole-9-ethylmethacrylate; allyl methacrylate; benzyl methacrylate; butyl methacrylate; cyclohexyl methacrylate; decyl methacrylate; di(ethylene glycol) ethyl ether methacrylate; di(ethylene glycol) methyl ether methacrylate; di(propylene glycol) allyl ether methacrylate, mixture of isomers; Disperse Red 1 methacrylate; Disperse Red 13 methacrylate; Disperse yellow 7 methacrylate; ethyl methacrylate; ethylene glycol dicyclopentenyl ether methacrylate; ethylene glycol methyl ether methacrylate; ethylene glycol phenyl ether methacrylate; furfuryl methacrylate; glycidyl methacrylate; glycol methacrylate; glycosyloxyethyl methacrylate; hexyl methacrylate; hydroxybutyl methacrylate, mixture of isomers; hydroxypropyl methacrylate; isobornyl methacrylate; isobutyl methacrylate; isodecyl methacrylate; lauryl methacrylate; methyl methacrylate; stearyl methacrylate; tert-butyl methacrylate; tetrahydrofurfuryl methacrylate; tridecyl methacrylate; trimethylsilyl methacrylate; vinyl methacrylate; glycerol propoxylate (1 PO/OH) triacrylate; pentaerythritol triacrylate; trimethylolpropane ethoxylate triacrylate; trimethylolpropane propoxylate triacrylate; trimethylolpropane triacrylate; di(trimethylolpropane) tetraacrylate; pentaerythritol tetraacrylate; dipentaerythritol pentaacrylate; ethoxylated pentaerythritol tetraacrylate; low viscosity dipentaerythritol pentaacrylate; pentaacrylate ester; pentaerythritol tetraacrylate; trimethylolpropane triacrylate; ethoxylated trimethylolpropane triacrylate; propoxylated glycerol triacrylate; pentaerythritol triacrylate; propoxylated glyceryl triacrylate; propoxylated trimethylolpropane triacrylate; trimethylolpropane trimethacrylate; tris(2-hydroxy ethyl)isocyanurate triacrylate; tris(2-hydroxy ethyl)isocyanurate triacrylate; polybutadiene diacrylate; and polybutadiene dimethacrylate. In certain particular embodiments, monomer is ethyl acrylate; vinyl acrylate; 1,3-butanediol diacrylate; dipentaerythritol pentaacrylate; tridecyl methacrylate; styrene; and 3,4-epoxycyclohexylmethyl 3′,4′-epoxycyclohexane carboxylate. In certain embodiments, the monomer is a polybutadiene di(meth)acrylate oligomer. In certain embodiments, the monomer is tricyclodecane dimethanol diacrylate. In certain embodiments, the monomer is tricyclodecane dimethanol dimethacrylate.

In certain embodiments, a fluorinated monomer is polymerized on skin based on the inventive skin treatment system. The fluorinated monomer comprises a functional group suitable for polymerization and at least one fluorine atom. Any functional group that can be polymerized using a free radical or ionic polymerization reaction can be used. Certain such functional groups are described. Typically, the functional group includes a degree of unsaturation (e.g., a double bond or triple bond). Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc.

The fluorinated monomer may range from including one fluorine atom to being perfluorinated. In certain embodiments, a functional group of the monomer is perfluorinated such as, for example, an alkyl, alkenyl, alkynyl, acyl, aryl, heteroaryl, heterocyclic, or carbocyclic moiety. In certain embodiments, the fluorinated monomer includes at least 1, 2, 3, 4, 5, 6, 7, 8, 9, 10, 11, 12, 13, 14, 15, 16, 17, 18, 19, or 20 fluorine atoms. In other embodiments, the fluorinated monomer contains at least 10, at least 15, at least 20, at least 25, at least 30, or at least 40 fluorine atoms. As would be appreciated by one of skill in this art, the larger the monomer the more fluorine atoms the monomer is likely to have. Furthermore, the monomer should include enough fluorine atoms so that the resulting polymer imparts the desired characteristics when polymerized on skin (e.g., appearance, robustness, feel).

In certain embodiments, at least 10%, 20%, 30%, 40%, 50%, 60%, 70%, 80%, 90%, 95%, or 99% of the total number of hydrogen and fluorine atoms are fluorine atoms in the fluorinated monomer. In certain embodiments, at least 50% of the total number of hydrogen and fluorine atoms are fluorine atoms in the fluorinated monomer. In certain embodiments, at least 80% of the total number of hydrogen and fluorine atoms are fluorine atoms in the fluorinated monomer. In certain embodiments, at least 90% of the total number of hydrogen and fluorine atoms are fluorine atoms in the fluorinated monomer. In certain embodiments, the fluorinated monomer is perfluorinated (i.e., all hydrogen atoms, or at least all non-exchangeable hydrogen atoms, are replaced with fluorine atoms).

In certain embodiments, the fluorinated monomer is a fluorinated alkene. In certain particular embodiments, the fluorinated alkene is monosubstituted. In other embodiments, the fluorinated alkene is disubstituted. Disubstituted fluorinated alkene may be either in the cis or trans configuration or a mixture thereof. In yet other embodiments, the fluorinated alkene is trisubstituted. The trisubstituted fluorinated alkene may be in either the E or Z configuration or a mixture thereof. In still other embodiments, the fluorinated alkene is tetrasubstituted. Again, various isomers are possible and are considered part of this invention. In certain embodiments, the fluorinated monomer is a fluorinated alkyne.

In certain embodiments, the monosubstituted fluorinated monomer is of the formula:

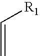

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and wherein R₁ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

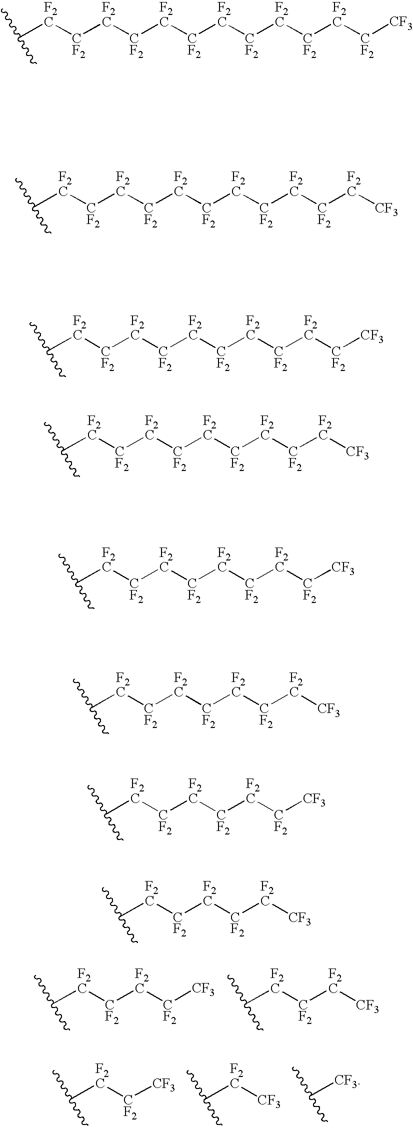

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

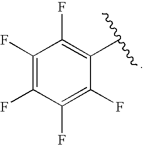

In certain particular embodiments, R₁ is of the formula:

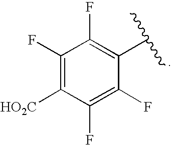

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}, wherein R_{A} comprises at least one fluorine atom. In other embodiments, R₁ is —CO₂R_{A}, wherein R_{A} comprises at least one fluorine atom. In certain particular embodiments, R₁ is —CO₂R_{A}, wherein R_{A} is one of the formulae:

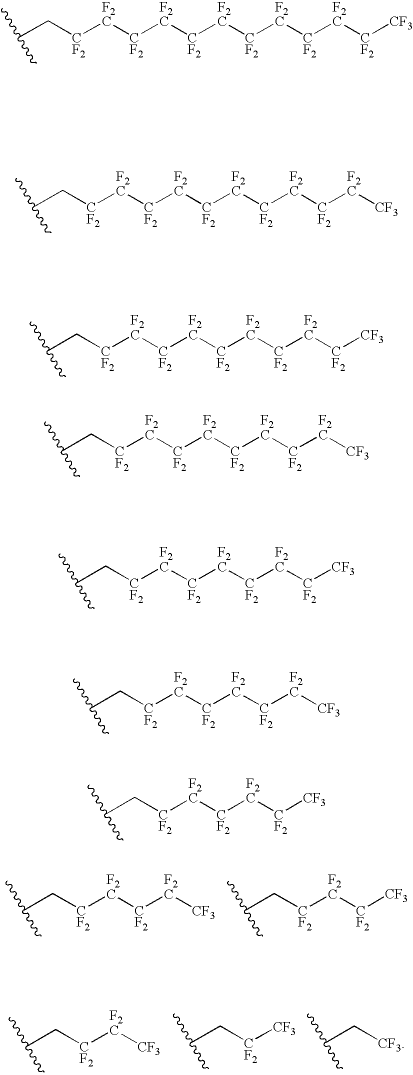

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments R₁ is —CO₂R_{A}, wherein R_{A} is of the formula:

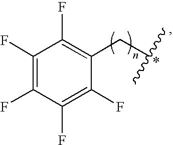

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

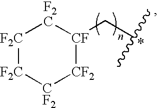

wherein n is an integer between 0 and 12, inclusive. In certain embodiments, n is 0. In certain embodiments, n is 1. In other embodiments, n is 2, 3, 4, 5, or 6.

In certain embodiments, the fluorinated monomer is a fluorinated acrylate of formula:

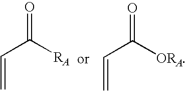

Exemplary monosubstituted fluorinated monomers include:

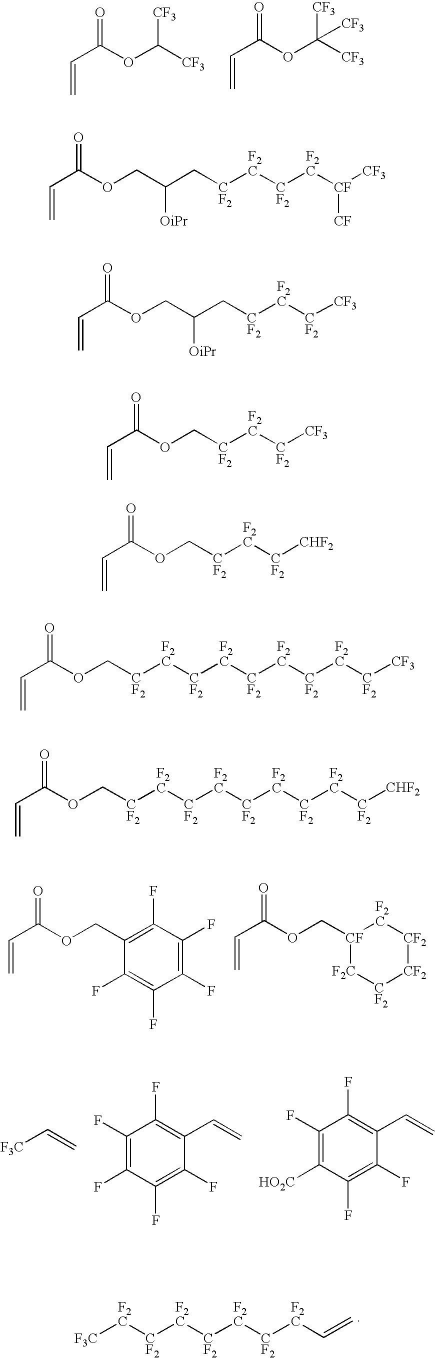

In certain embodiments, the disubstituted fluorinated monomer is of one of the formulae:

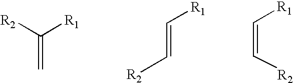

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and wherein R₁ or R₂ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

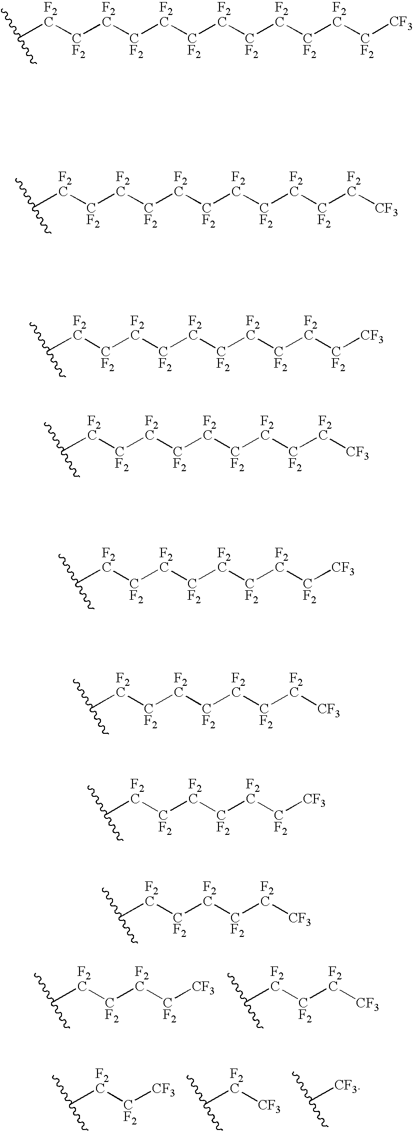

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

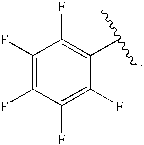

In certain particular embodiments, R₁ is of the formula:

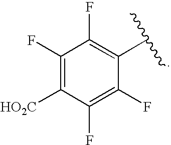

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

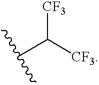

In other embodiments, R_{A} is t-butyl. In certain particular embodiments, R₁ is —CO₂R_{A}, wherein R_{A} is one of the formulae:

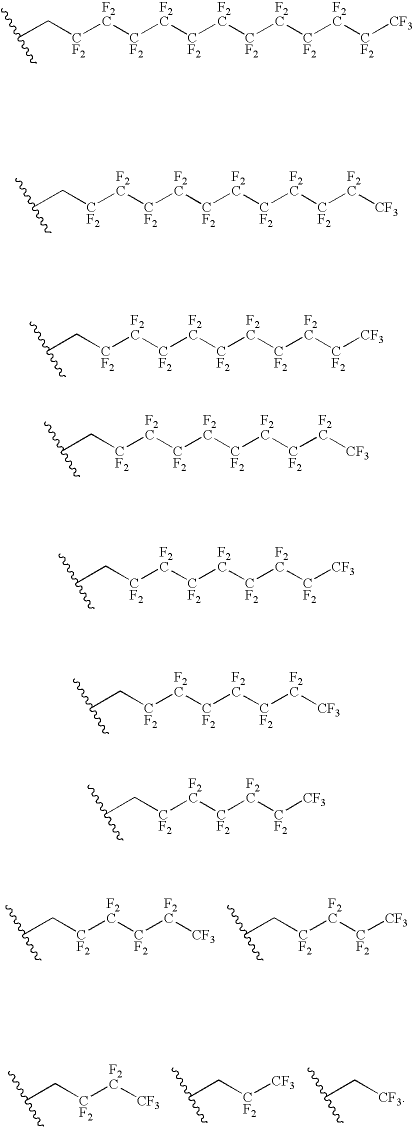

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

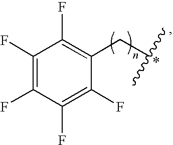

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

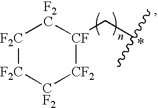

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, only one of R₁ and R₂ includes fluorine atoms. In other embodiments, both R₁ and R₂ include fluorine atoms. In certain embodiments, R₂ is fluorine. In other embodiments, R₂ is substituted or unsubstituted, branched or unbranched aliphatic. In yet other embodiments, R₂ is C₁-C₆ alkyl. In certain embodiments, R₂ is a perfluorinated alkyl moiety. In certain particular embodiments, R₂ is methyl. In certain embodiments R₂ is —CF₃, —CHF₂, or —CH₂F. In certain embodiments, R₂ is a fluorine-substituted aryl or heteroaryl moiety. In certain embodiments, R₂ is a fluorine-substituted phenyl moiety. In certain particular embodiments, R₂ is a perfluorinated phenyl moiety.

In certain embodiments, R₁ is —CO₂R_{A}, and R₂ is fluorine. In other embodiments, R₁ is —CO₂R_{A}, and R₂ is C₁-C₆ alkyl, optionally substituted with fluorine. In other embodiments, R₁ is —CO₂R_{A}, and R₂ is methyl. In yet other embodiments, R₁ is —CO₂R_{A}, and R₂ is —CF₃.

In certain embodiments, the fluorinated monomer is a fluorinated methacrylate of formula:

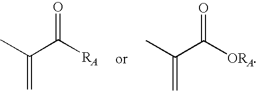

In certain embodiments, the fluorinated monomer is a fluorinated acrylate of formula:

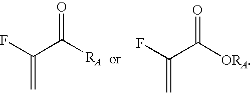

In certain embodiments, the fluorinated monomer is a fluorinated methacrylate of formula:

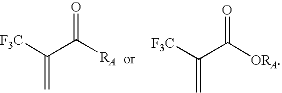

In certain embodiments, the fluorinated monomer is a fluorinated crotonate of formula:

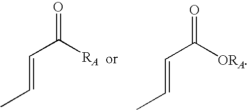

In certain embodiments, the fluorinated monomer is a fluorinated crontonate of formula:

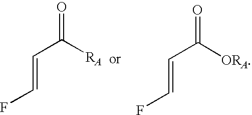

In certain embodiments, the fluorinated monomer is a fluorinated crotonate of formula:

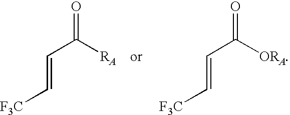

Exemplary disubstituted fluorinated monomers include:

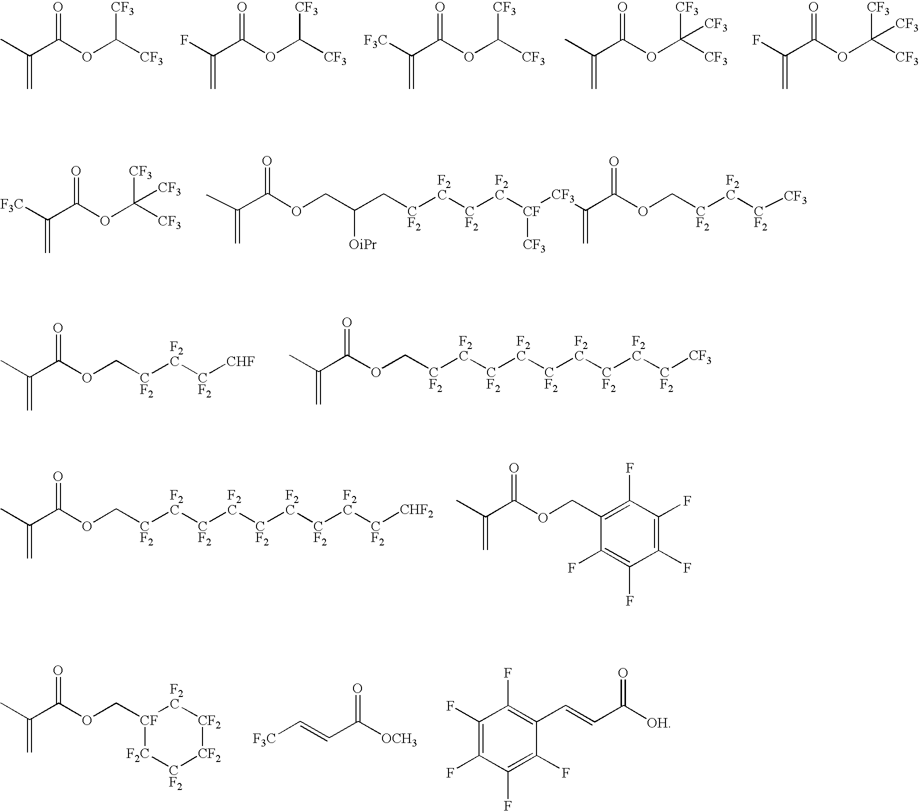

In certain embodiments, the trisubstituted fluorinated monomer is of one of the formulae:

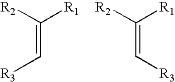

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and wherein R₁, R₂, or R₃ comprises at least one fluorine atom.

In certain embodiments, R₁ contains more than 1, 2, 3, 4, 5, 10, 15, 20, or 25 fluorine atoms. In certain embodiments, R₁ is fluorine. In other embodiments, R₁ is a fluorinated, substituted or unsubstituted, branched or unbranched aliphatic moiety. In certain embodiments, R₁ is a fluorinated alkyl moiety. In certain embodiments, R₁ is of one of the formulae:

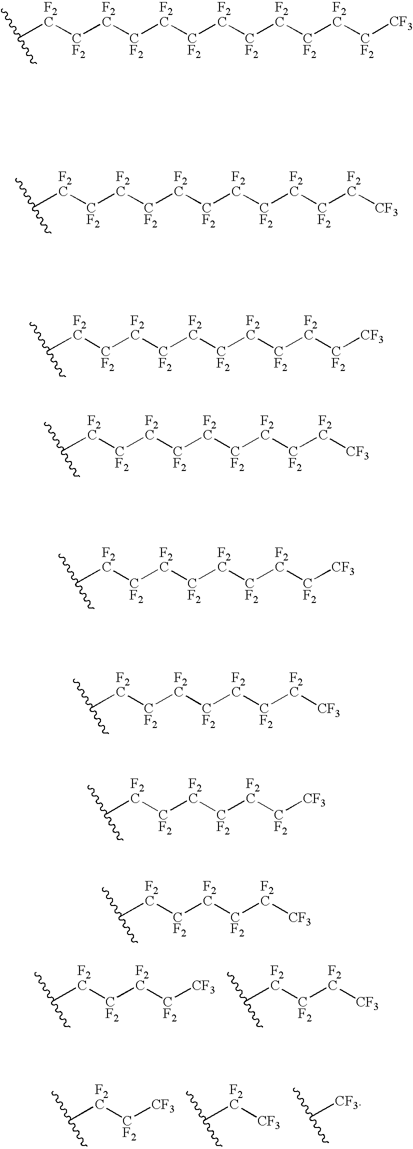

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

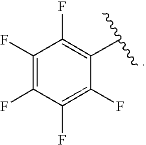

In certain particular embodiments, R₁ is of the formula:

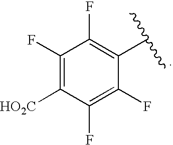

In certain particular embodiments, R₁ is a fluorinated, substituted or unsubstituted phenyl moiety. In certain embodiments, R₁ is fluorinated phenyl (e.g., a phenyl ring with 1, 2, 3, 4, or 5 fluorine substituents). In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

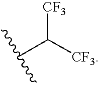

In other embodiments, R_{A} is t-butyl. In certain particular embodiments, R₁ is —CO₂R_{A}, wherein R_{A} is one of the formulae:

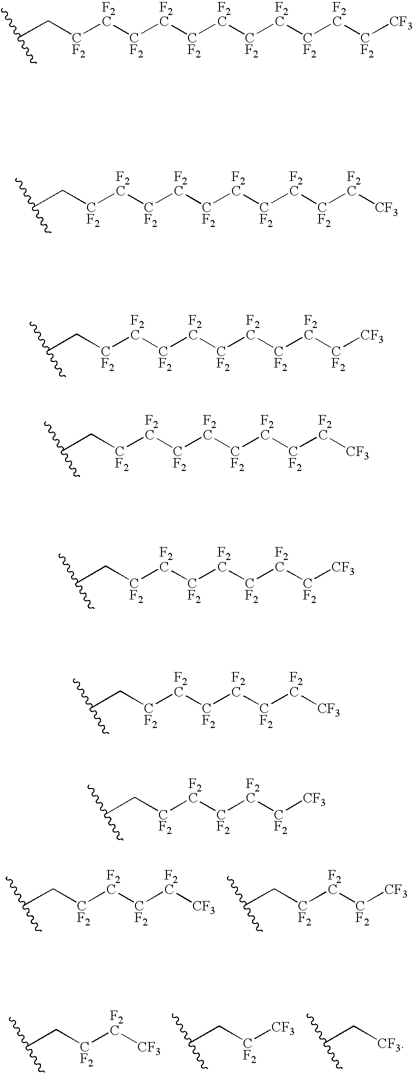

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

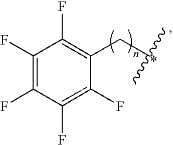

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

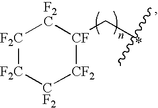

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, R₂ is fluorine. In other embodiments, R₂ is substituted or unsubstituted, branched or unbranched aliphatic. In yet other embodiments, R₂ is C₁-C₆ alkyl, optionally substituted with a fluorine. In certain embodiments, R₂ is a perfluorinated C₁-C₆ alkyl moiety. In certain particular embodiments, R₂ is methyl. In certain embodiments R₂ is —CF₃, —CHF₂, or —CH₂F.

In certain embodiments, R₂ is of one of the formulae:

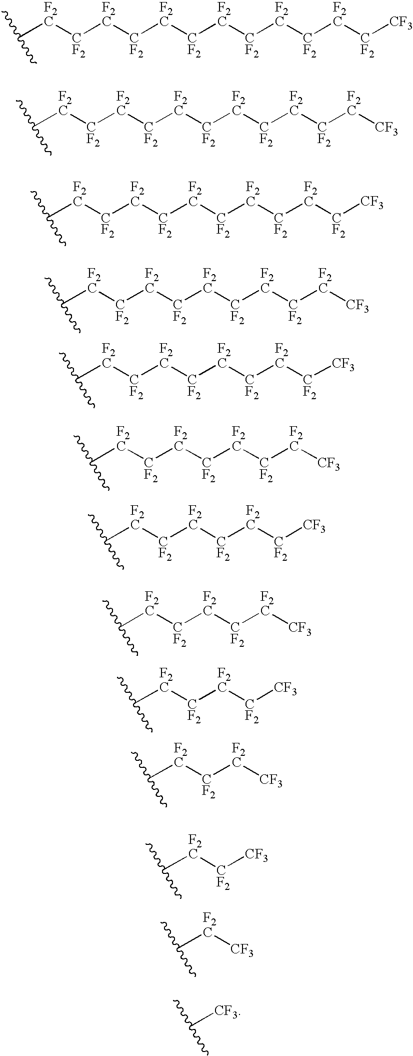

As would be appreciated by one of skill in this art, any of the above perfluorinated alkyl groups may be partially fluorinated, substituted, branched, unsaturated, and/or cyclic. In yet other embodiments, R₂ is a fluorinated, substituted or unsubstituted, branched or unbranched heteroaliphatic moiety. In still other embodiments, R₂ is a fluorinated, substituted or unsubstituted acyl moiety. In other embodiments, R₂ is a fluorinated, substituted or unsubstituted aryl moiety. In certain particular embodiments, R₂ is of the formula:

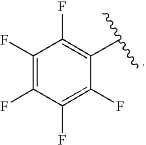

In certain particular embodiments, R₂ is of the formula:

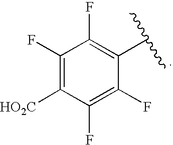

In certain particular embodiments, R₂ is a fluorinated, substituted or unsubstituted phenyl moiety. In certain embodiments, R₂ is fluorinated phenyl (e.g., a phenyl ring with 1, 2, 3, 4, or 5 fluorine substituents). In other embodiments, R₂ is a fluorinated, substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain particular embodiments, R_{B} is —CF₃. In certain embodiments, R_{B} is

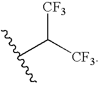

In other embodiments, R_{B} is t-butyl. In certain particular embodiments, R₂ is —CO₂R_{B}, wherein R_{B} is one of the formulae:

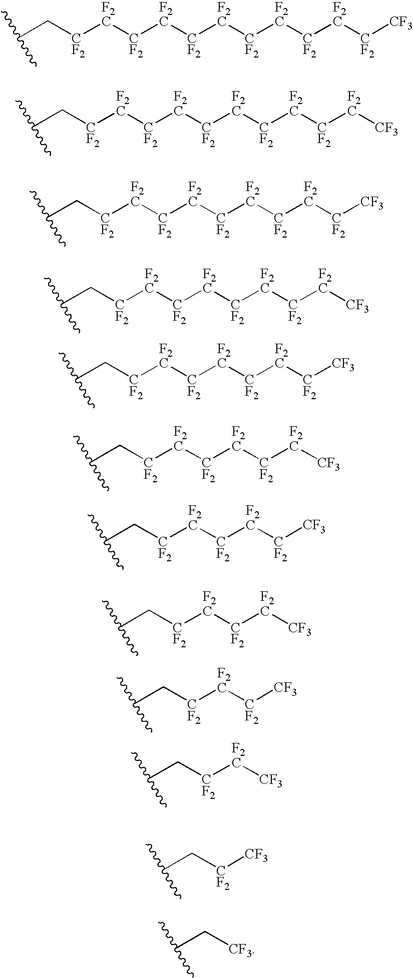

As would be appreciated by one of skill in this art, any of the above fluorinated alkyl groups may be partially fluorinated, substituted, branched, unsaturated, and/or cyclic. In certain particular embodiments, R₂ is —CO₂R_{B}, wherein R_{B} is fluorinated aryl or fluorinated arylalkyl. In certain particular embodiments, R₂ is —CO₂R_{B}, wherein R_{B} is of the formula:

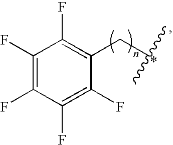

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

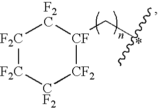

wherein n is an integer between 0 and 12, inclusive. In certain embodiments, n is 0. In certain embodiments, n is 1. In other embodiments, n is 2, 3, 4, 5, or 6.

In certain embodiments, R₃ includes more than 1, 2, 3, 4, 5, 10, 15, 20, or 25 fluorine atoms. In certain embodiments, only one of R₁, R₂, and R₃ includes fluorine atoms. In certain other embodiments, only two of R₁, R₂, and R₃ includes fluorine atoms. In other embodiments, all of R₁, R₂, and R₃ include fluorine atoms. In certain embodiments, R₃ is fluorine. In other embodiments, R₃ is substituted or unsubstituted, branched or unbranched aliphatic. In yet other embodiments, R₃ is C₁-C₆ alkyl, optionally substituted with a fluorine. In certain embodiments, R₃ is a perfluorinated C₁-C₆ alkyl moiety. In certain particular embodiments, R₃ is methyl. In certain embodiments R₃ is —CF₃, —CHF₂, or —CH₂F.

In certain embodiments, R₃ is of one of the formulae:

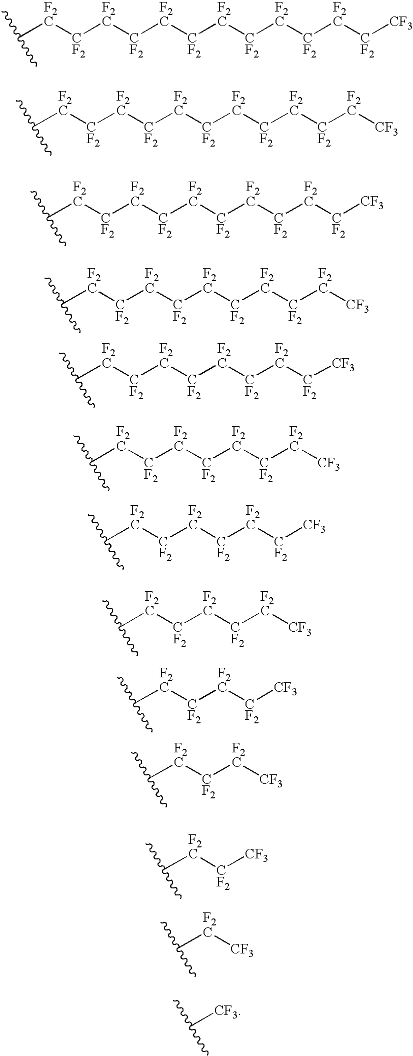

As would be appreciated by one of skill in this art, any of the above perfluorinated alkyl groups may be partially fluorinated, substituted, branched, unsaturated, and/or cyclic. In yet other embodiments, R₃ is a fluorinated, substituted or unsubstituted, branched or unbranched heteroaliphatic moiety. In still other embodiments, R₃ is a fluorinated, substituted or unsubstituted acyl moiety. In other embodiments, R₃ is a fluorinated, substituted or unsubstituted aryl moiety. In certain particular embodiments, R₃ is of the formula:

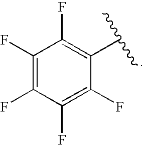

In certain particular embodiments, R₃ is of the formula:

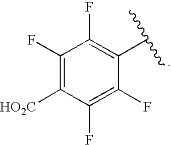

In certain particular embodiments, R₃ is a fluorinated, substituted or unsubstituted phenyl moiety. In certain embodiments, R₃ is fluorinated phenyl (e.g., a phenyl ring with 1, 2, 3, 4, or 5 fluorine substituents). In other embodiments, R₃ is a fluorinated, substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain particular embodiments, R_{C} is —CF₃. In certain embodiments, R_{C} is

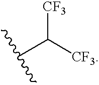

In other embodiments, R_{C} is t-butyl. In certain particular embodiments, R₃ is —CO₂R_{A}, wherein R_{C} is one of the formulae:

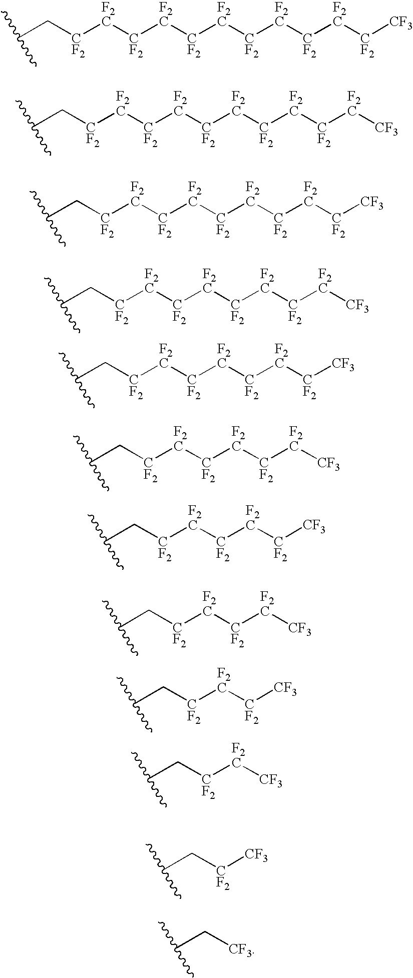

As would be appreciated by one of skill in this art, any of the above fluorinated alkyl groups may be partially fluorinated, substituted, branched, unsaturated, and/or cyclic. In certain particular embodiments, R₃ is —CO₂R_{C}, wherein R_{C} is fluorinated aryl or fluorinated arylalkyl. In certain particular embodiments, R₃ is —CO₂R_{C}, wherein R_{C} is of the formula:

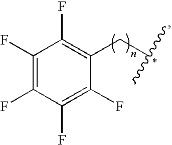

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

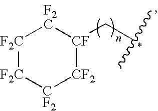

wherein n is an integer between 0 and 12, inclusive. In certain embodiments, n is 0. In certain embodiments, n is 1. In other embodiments, n is 2, 3, 4, 5, or 6.

In certain embodiments, R₁ is —CO₂R_{A}, and R₂ and R₃ are both fluorine. In other embodiments, R₁ is —CO₂R_{A}, and R₂ and R₃ are both methyl. In yet other embodiments, R₁ is —CO₂R_{A}, and R₂ and R₃ are both —CF₃. In certain embodiments, at least one of R₁, R₂, and R₃ is fluorine. In other embodiments, at least two of R₁, R₂, and R₃ are fluorine.

Exemplary trisubstituted fluorinated monomers include:

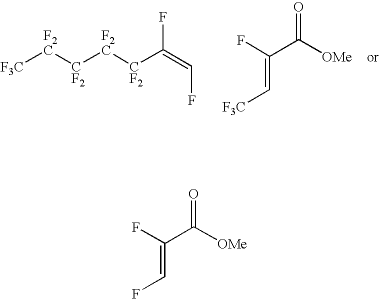

In certain embodiments, the tetrasubstituted fluorinated monomer is of one of the formulae:

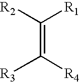

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —C(═O)N(R_{C})₂; —CN; —SCN; —SR_{C}; —SOR_{C}; —SO₂R_{C}; —NO_{C}; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —C(═O)N(R_{D})₂; —CN; —SCN; —SR_{D}; —SOR_{D}; —SO₂R_{D}; —NO_{D}; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁, R₂, R₃, or R₄ comprises at least one fluorine atom.

In certain embodiments, R₁ is fluorine. In other embodiments, R₁ is a fluorinated, substituted or unsubstituted, branched or unbranched aliphatic moiety. In certain embodiments, R₁ is of one of the formulae:

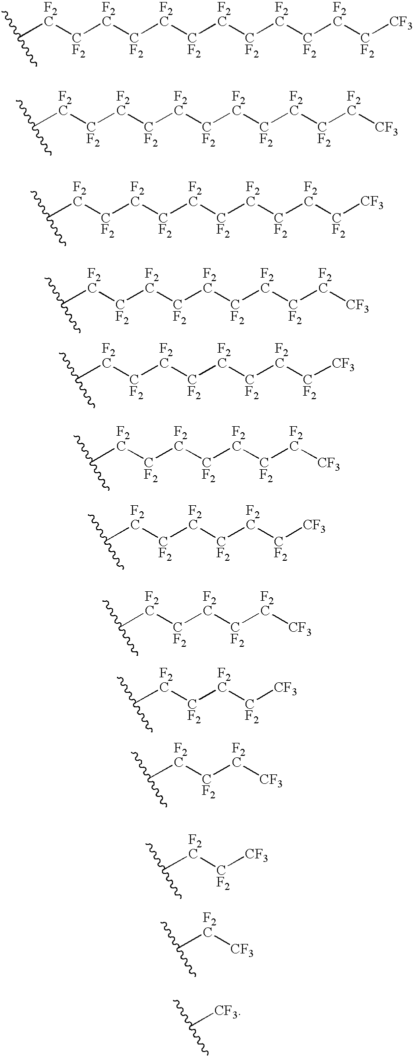

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

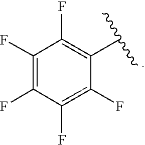

In certain particular embodiments, R₁ is of the formula:

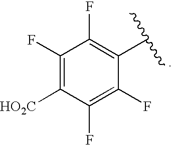

In certain particular embodiments, R₁ is a fluorinated, substituted or unsubstituted phenyl moiety. In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

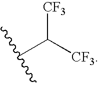

In other embodiments, R_{A} is t-butyl. In certain particular embodiments, R₁ is —CO₂R_{A}, wherein R_{A} is one of the formulae:

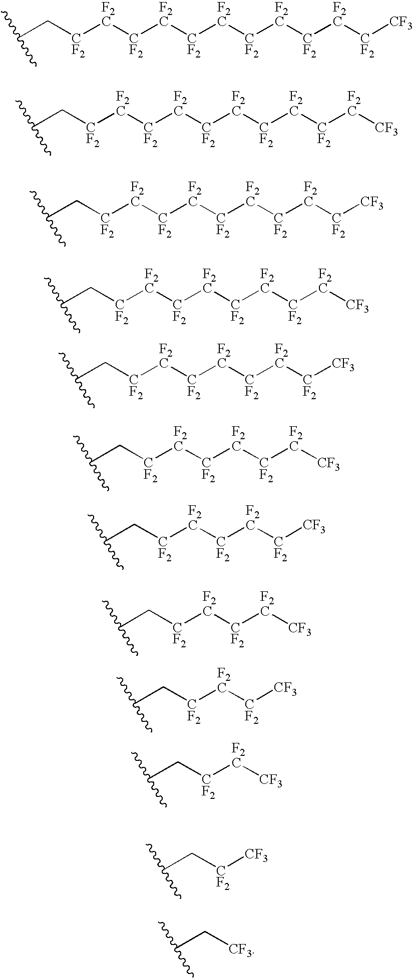

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

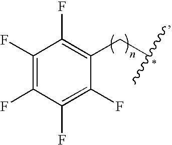

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

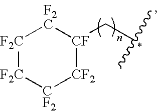

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, R₂ is fluorine. In other embodiments, R₂ is substituted or unsubstituted, branched or unbranched aliphatic. In yet other embodiments, R₂ is C₁-C₆ alkyl, optionally substituted with a fluorine. In certain embodiments, R₂ is a perfluorinated C₁-C₆ alkyl moiety. In certain particular embodiments, R₂ is methyl. In certain embodiments R₂ is —CF₃, —CHF₂, or —CH₂F.

In certain embodiments, R₂ is of one of the formulae:

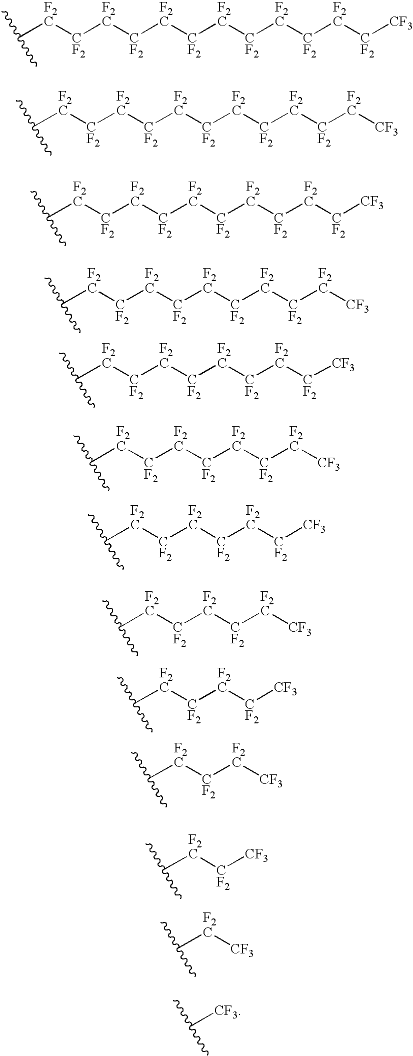

As would be appreciated by one of skill in this art, any of the above perfluorinated alkyl groups may be partially fluorinated, substituted, branched, unsaturated, and/or cyclic. In yet other embodiments, R₂ is a fluorinated, substituted or unsubstituted, branched or unbranched heteroaliphatic moiety. In still other embodiments, R₂ is a fluorinated, substituted or unsubstituted acyl moiety. In other embodiments, R₂ is a fluorinated, substituted or unsubstituted aryl moiety. In certain particular embodiments, R₂ is of the formula:

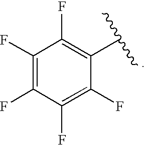

In certain particular embodiments, R₂ is of the formula:

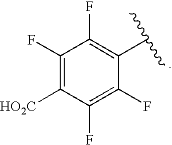

In certain particular embodiments, R₂ is a fluorinated, substituted or unsubstituted phenyl moiety. In certain embodiments, R₂ is fluorinated phenyl (e.g., a phenyl ring with 1, 2, 3, 4, or 5 fluorine substituents). In other embodiments, R₂ is a fluorinated, substituted or unsubstituted heteroaryl moiety. In certain embodiments, R₂ is —C(═O)R_{B}. In other embodiments, R₂ is —CO₂R_{B}. In certain embodiments, R_{B} is C₁-C₆ alkyl. In certain particular embodiments, R_{B} is methyl. In certain particular embodiments, R_{B} is —CF₃. In certain embodiments, R_{B} is

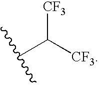

In other embodiments, R_{B} is t-butyl. In certain particular embodiments, R₂ is —CO₂R_{B}, wherein R_{B} is one of the formulae:

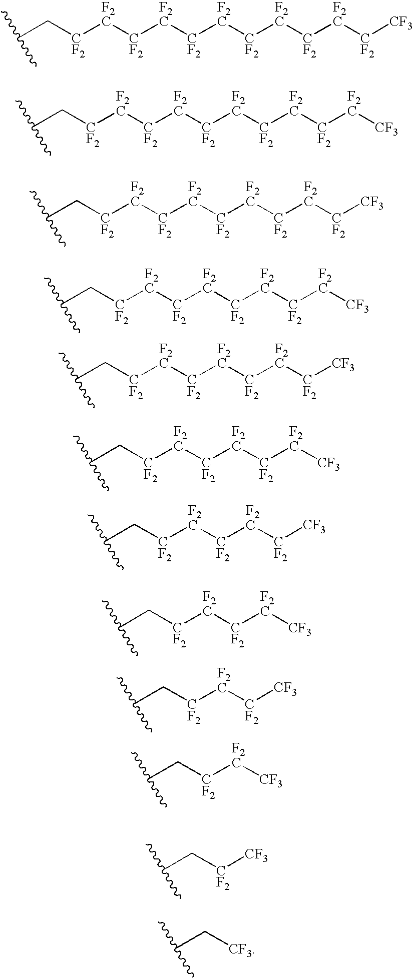

As would be appreciated by one of skill in this art, any of the above fluorinated alkyl groups may be partially fluorinated, substituted, branched, unsaturated, and/or cyclic. In certain particular embodiments, R₂ is —CO₂R_{B}, wherein R_{B} is fluorinated aryl or fluorinated arylalkyl. In certain particular embodiments, R₂ is —CO₂R_{B}, wherein R_{B} is of the formula:

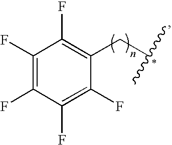

wherein n is an integer between 0 and 12, inclusive. In certain embodiments, n is 0. In certain embodiments, n is 1. In other embodiments, n is 2, 3, 4, 5, or 6. In certain particular embodiments, R₂ is —CO₂R_{B}, wherein R_{B} is of the formula:

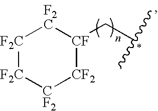

wherein n is an integer between 0 and 12, inclusive. In certain embodiments, n is 0. In certain embodiments, n is 1. In other embodiments, n is 2, 3, 4, 5, or 6.

In certain embodiments, R₃ includes more than 1, 2, 3, 4, 5, 10, 15, 20, or 25 fluorine atoms. In certain embodiments, R₃ is fluorine. In other embodiments, R₃ is substituted or unsubstituted, branched or unbranched aliphatic. In yet other embodiments, R₃ is C₁-C₆ alkyl, optionally substituted with a fluorine. In certain embodiments, R₃ is a perfluorinated C₁-C₆ alkyl moiety. In certain particular embodiments, R₃ is methyl. In certain embodiments R₃ is —CF₃, —CHF₂, or —CH₂F.

In certain embodiments, R₃ is of one of the formulae:

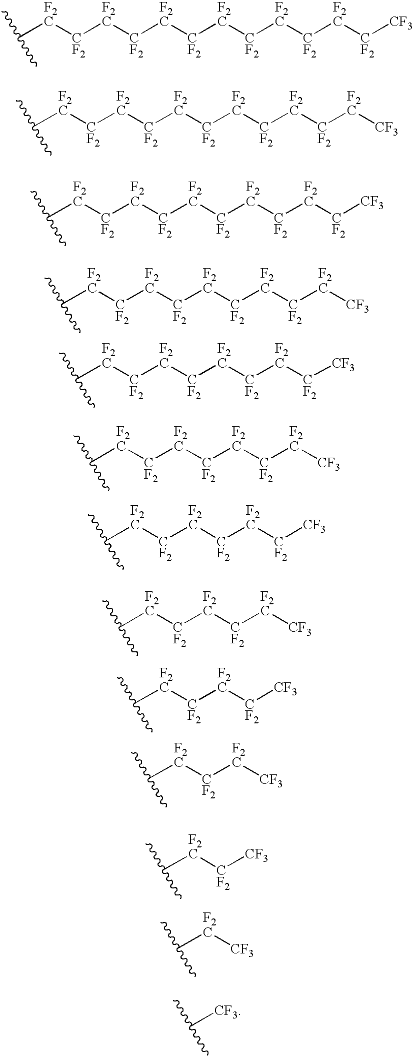

As would be appreciated by one of skill in this art, any of the above perfluorinated alkyl groups may be partially fluorinated, substituted, branched, unsaturated, and/or cyclic. In yet other embodiments, R₃ is a fluorinated, substituted or unsubstituted, branched or unbranched heteroaliphatic moiety. In still other embodiments, R₃ is a fluorinated, substituted or unsubstituted acyl moiety. In other embodiments, R₃ is a fluorinated, substituted or unsubstituted aryl moiety. In certain particular embodiments, R₃ is of the formula:

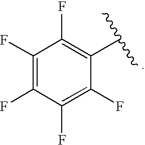

In certain particular embodiments, R₃ is of the formula:

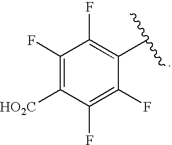

In certain particular embodiments, R₃ is a fluorinated, substituted or unsubstituted phenyl moiety. In certain embodiments, R₃ is fluorinated phenyl (e.g., a phenyl ring with 1, 2, 3, 4, or 5 fluorine substituents). In other embodiments, R₃ is a fluorinated, substituted or unsubstituted heteroaryl moiety. In certain embodiments, R₃ is —C(═O)R_{C}. In other embodiments, R₃ is —CO₂R_{C}. In certain embodiments, R_{C} is C₁-C₆ alkyl. In certain particular embodiments, R_{C} is methyl. In certain particular embodiments, R_{C} is —CF₃. In certain embodiments, R_{C} is

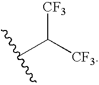

In other embodiments, R_{C} is t-butyl. In certain particular embodiments, R₃ is —CO₂R_{C}, wherein R_{C} is one of the formulae:

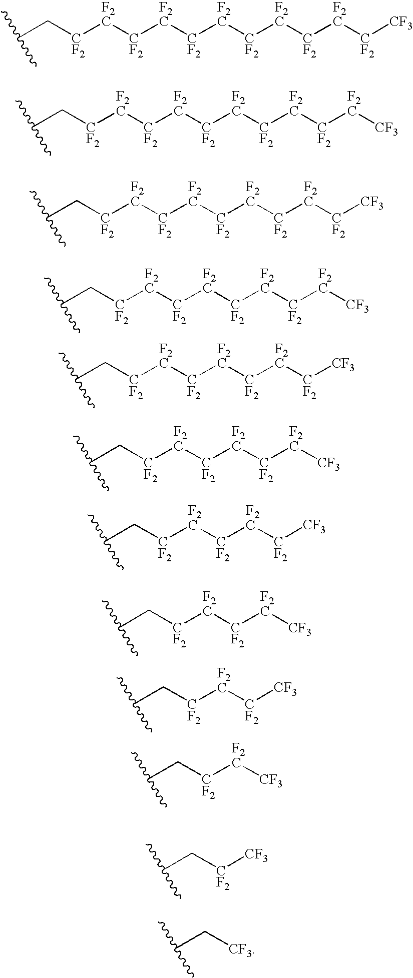

As would be appreciated by one of skill in this art, any of the above fluorinated alkyl groups may be partially fluorinated, substituted, branched, unsaturated, and/or cyclic. In certain particular embodiments, R₃ is —CO₂R_{C}, wherein R_{C} is fluorinated aryl or fluorinated arylalkyl. In certain particular embodiments, R₃ is —CO₂R_{C}, wherein R_{C} is of the formula:

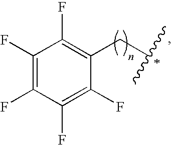

wherein n is an integer between 0 and 12, inclusive. In certain embodiments, n is 0. In certain embodiments, n is 1. In other embodiments, n is 2, 3, 4, 5, or 6. In certain particular embodiments, R₃ is —CO₂R_{C}, wherein R_{C} is of the formula:

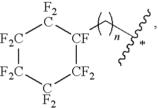

wherein n is an integer between 0 and 12, inclusive. In certain embodiments, n is 0. In certain embodiments, n is 1. In other embodiments, n is 2, 3, 4, 5, or 6.

In certain embodiments, R₄ includes more than 1, 2, 3, 4, 5, 10, 15, 20, or 25 fluorine atoms. In certain embodiments, only one of R₁, R₂, R₃, and R₄ includes fluorine atoms. In certain other embodiments, only two of R₁, R₂, R₃, and R₄ includes fluorine atoms. In certain other embodiments, only three of R₁, R₂, R₃, and R₄ includes fluorine atoms. In other embodiments, all of R₁, R₂, R₃, and R₄ include fluorine atoms. In certain embodiments, R₄ is fluorine. In other embodiments, R₄ is substituted or unsubstituted, branched or unbranched aliphatic. In yet other embodiments, R₄ is C₁-C₆ alkyl, optionally substituted with a fluorine. In certain embodiments, R₄ is a perfluorinated C₁-C₆ alkyl moiety. In certain particular embodiments, R₄ is methyl. In certain embodiments R₄ is —CF₃, —CHF₂, or —CH₂F.

In certain embodiments, R₄ is of one of the formulae:

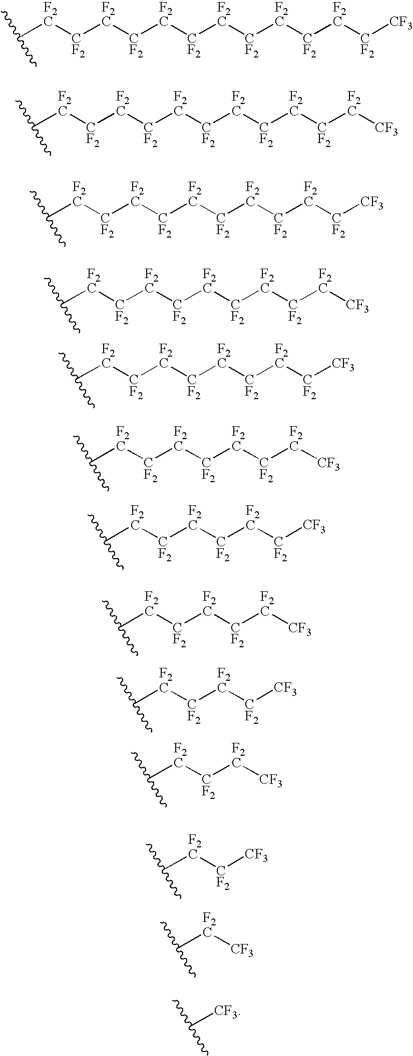

As would be appreciated by one of skill in this art, any of the above perfluorinated alkyl groups may be partially fluorinated, substituted, branched, unsaturated, and/or cyclic. In yet other embodiments, R₄ is a fluorinated, substituted or unsubstituted, branched or unbranched heteroaliphatic moiety. In still other embodiments, R₄ is a fluorinated, substituted or unsubstituted acyl moiety. In other embodiments, R₄ is a fluorinated, substituted or unsubstituted aryl moiety. In certain particular embodiments, R₄ is of the formula:

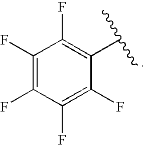

In certain particular embodiments, R₄ is of the formula:

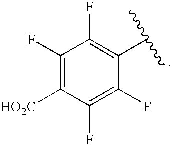

In certain particular embodiments, R₄ is a fluorinated, substituted or unsubstituted phenyl moiety. In certain embodiments, R₄ is fluorinated phenyl (e.g., a phenyl ring with 1, 2, 3, 4, or 5 fluorine substituents). In other embodiments, R₄ is a fluorinated, substituted or unsubstituted heteroaryl moiety. In certain embodiments, R₄ is —C(═O)R_{D}. In other embodiments, R₄ is —CO₂R_{D}. In certain embodiments, R_{D} is C₁-C₆ alkyl. In certain particular embodiments, R_{D} is methyl. In certain particular embodiments, R_{D} is —CF₃. In certain embodiments, R_{D} is

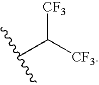

In other embodiments, R_{D} is t-butyl. In certain particular embodiments, R₄ is —CO₂R_{D}, wherein R_{D} is one of the formulae:

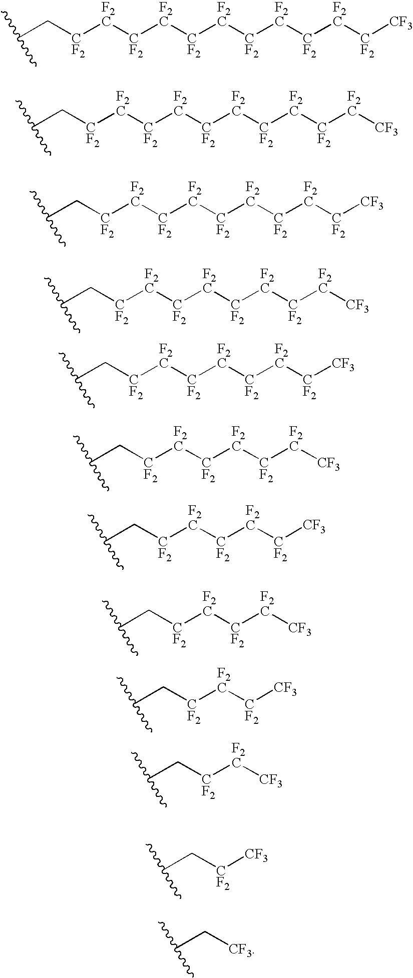

As would be appreciated by one of skill in this art, any of the above fluorinated alkyl groups may be partially fluorinated, substituted, branched, unsaturated, and/or cyclic. In certain particular embodiments, R₄ is —CO₂R_{D}, wherein R_{D} is fluorinated aryl or fluorinated arylalkyl. In certain particular embodiments, R₄ is —CO₂R_{D}, wherein R_{D} is of the formula:

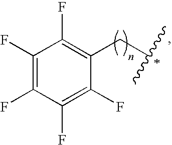

wherein n is an integer between 0 and 12, inclusive. In certain embodiments, n is 0. In certain embodiments, n is 1. In other embodiments, n is 2, 3, 4, 5, or 6. In certain particular embodiments, R₄ is —CO₂R_{D}, wherein R_{D} is of the formula:

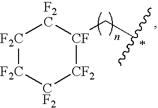

wherein n is an integer between 0 and 12, inclusive. In certain embodiments, n is 0. In certain embodiments, n is 1. In other embodiments, n is 2, 3, 4, 5, or 6.

In certain embodiments, R₁ is —CO₂R_{A}, and R₂ and R₃ are both fluorine. In other embodiments, R₁ is —CO₂R_{A}, and R₂ and R₃ are both methyl. In yet other embodiments, R₁ is —CO₂R_{A}, and R₂ and R₃ are both —CF₃. In certain embodiments, at least one of R₁, R₂, R₃, and R₄ is fluorine. In other embodiments, at least two of R₁, R₂, R₃, and R₄ are fluorine. In other embodiments, at least three of R₁, R₂, R₃, and R₄ are fluorine.

Exemplary tetrasubstituted fluorinated monomers include:

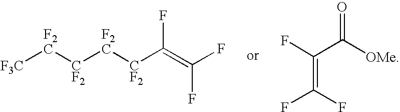

In certain embodiments, the fluorinated monomer is a fluorinated diacrylate or dimethacrylate. In certain embodiments, the fluorinated diacrylate is of the formula:

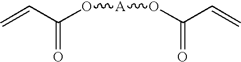

wherein A is a fluorinated linker. In certain embodiments, the fluorinated difluoroacrylate is of the formula:

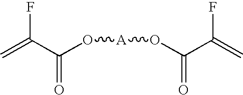

wherein A is a fluorinated linker. In certain embodiments, the fluorinated dimethacrylate is of the formula:

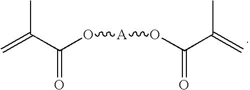

wherein A is a fluorinated linker. In certain embodiments, the fluorinated dimethacrylate is of the formula:

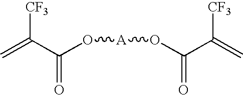

wherein A is a fluorinated linker. In certain embodiments, A is a fluorinated, substituted or unsubstituted, branched or unbranched, cyclic or acyclic aliphatic; fluorinated, substituted or unsubstituted, branched or unbranched, cyclic or acyclic heteroaliphatic; fluorinated, substituted or unsubstituted, aryl; or fluorinated, substituted or unsubstituted, heteroaryl. In certain embodiments, the linker A is a fluorinated alkyl linker. In certain embodiments, the linker A is of one of the formulae:

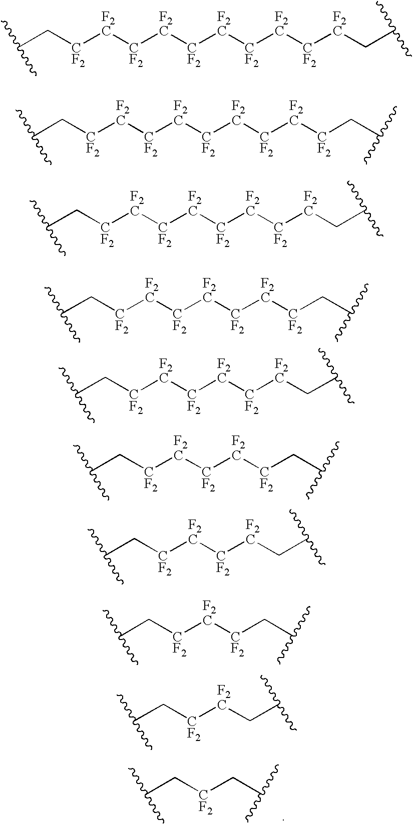

As would be appreciated by one of skill in this art, any of the above fluorinated alkyl groups may be partially fluorinated, substituted, branched, unsaturated, and/or cyclic. In other embodiments, the linker A is of one of the formulae:

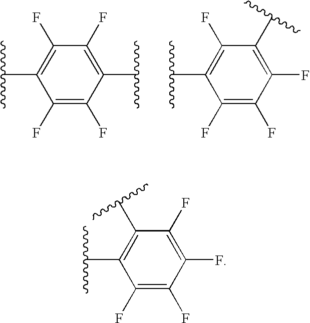

Exemplary diacrylate and dimethacrylates include:

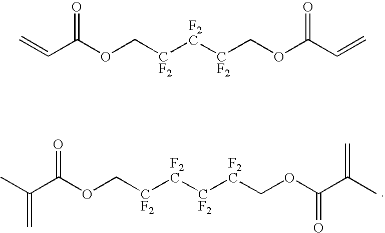

In certain embodiments, the fluorinated monomer is a fluorinated triacrylate or trimethacrylate. In certain embodiments, the fluorinated monomer is of the formula:

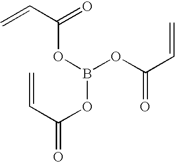

wherein B is fluorinated linker.

In other embodiments, the fluorinated monomer is of the formula:

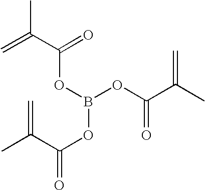

wherein B is fluorinated linker.

In still other embodiments, the fluorinated monomer is of the formula:

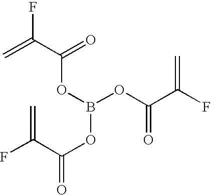

wherein B is fluorinated linker.

In still other embodiments, the fluorinated monomer is of the formula:

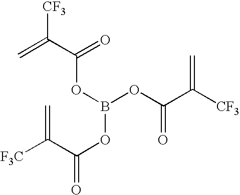

wherein B is fluorinated linker.

In certain embodiments, linker B is a fluorinated, substituted or unsubstituted, branched or unbranched, cyclic or acyclic aliphatic; fluorinated, substituted or unsubstituted, branched or unbranched, cyclic or acyclic heteroaliphatic; fluorinated, substituted or unsubstituted, aryl; or fluorinated, substituted or unsubstituted, heteroaryl. In certain embodiments, the linker B is a branched, fluorinated alkyl linker. In certain embodiments, the linker B is a fluorinated aryl linker. In certain embodiments, the linker B is of the formula:

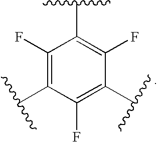

An exemplary trimethacrylate is of the formula:

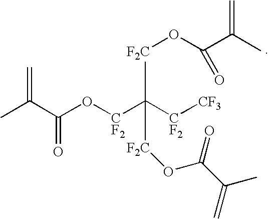

In certain embodiments, the fluorinated monomer is a fluorinated tetraacrylate or tetramethacrylate. Tetraacrylates may be prepared by reacting diacrylates or dimethacrylates with a diamine. An exemplary tetramethacrylate is of the formula:

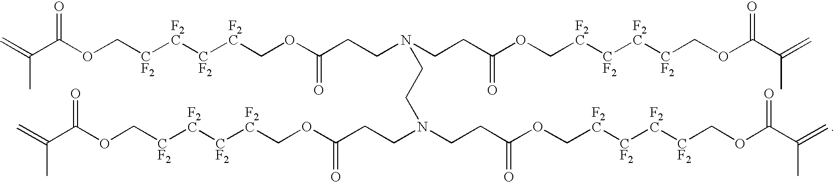

In other embodiments, the fluorinated monomer is a fluorinated pentaacrylate or pentamethacrylate. In still other embodiments, the fluorinated monomer is an even higher acrylate or methacrylate.

In certain embodiments, the fluorinated monomer is a fluorinated alkyne. In certain embodiments, the fluorinated alkynyl monomer is of the formula:

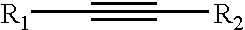

wherein

R₁ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —C(═O)N(R_{A})₂; —CN; —SCN; —SR_{A}; —SOR_{A}; —SO₂R_{A}; —NO_{A}; —N(R_{C})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —C(═O)N(R_{B})₂; —CN; —SCN; —SR_{B}; —SOR_{B}; —SO₂R_{B}; —NO_{B}; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

wherein R₁ and R₂ comprises at least one fluorine atom.

In certain embodiments, R₁ is fluorine. In certain embodiments, R₁ is hydrogen. In other embodiments, R₁ is a fluorinated, substituted or unsubstituted, branched or unbranched aliphatic moiety. In certain embodiments, R₁ is of one of the formulae:

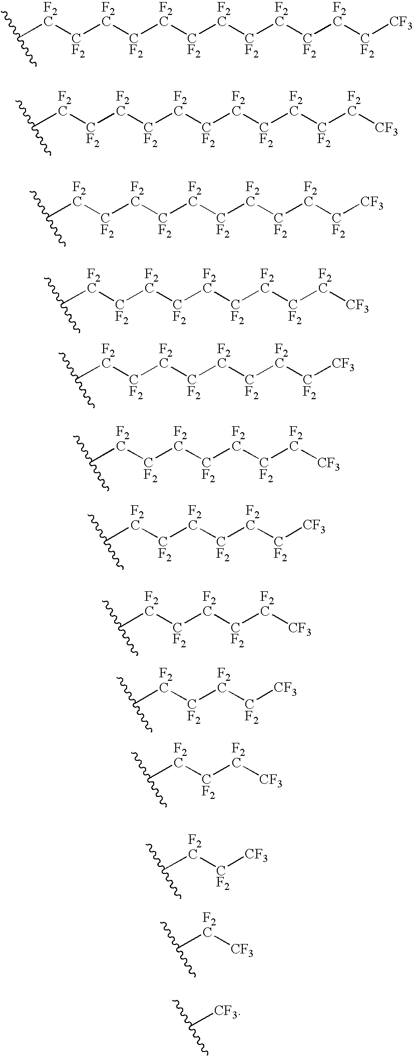

As would be appreciated by one of skill in this art, any of the above perfluorinated alkyl groups may be partially fluorinated, substituted, branched, unsaturated, and/or cyclic. In yet other embodiments, R₁ is a fluorinated, substituted or unsubstituted, branched or unbranched heteroaliphatic moiety. In still other embodiments, R₁ is a fluorinated, substituted or unsubstituted acyl moiety. In other embodiments, R₁ is a fluorinated, substituted or unsubstituted aryl moiety. In certain particular embodiments, R₁ is of the formula:

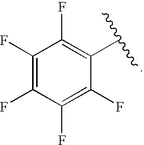

In certain particular embodiments, R₁ is of the formula:

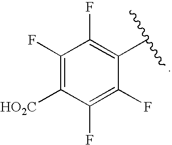

In certain particular embodiments, R₁ is a fluorinated, substituted or unsubstituted phenyl moiety. In other embodiments, R₁ is a fluorinated, substituted or unsubstituted heteroaryl moiety. In certain embodiments, R₁ is —C(═O)R_{A}. In other embodiments, R₁ is —CO₂R_{A}. In certain embodiments, R_{A} is C₁-C₆ alkyl. In certain particular embodiments, R_{A} is methyl. In certain particular embodiments, R_{A} is —CF₃. In certain embodiments, R_{A} is

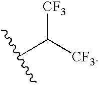

In other embodiments, R_{A} is t-butyl. In certain particular embodiments, R₁ is —CO₂R_{A}, wherein R_{A} is one of the formulae:

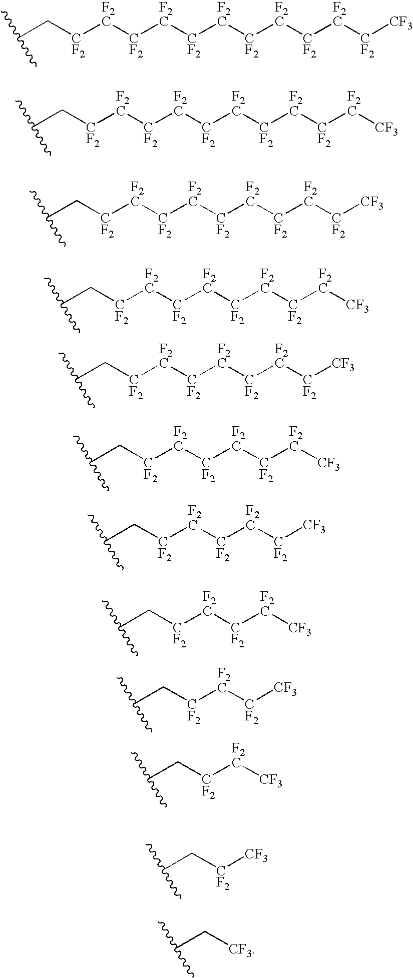

As would be appreciated by one of skill in this art, any of the above fluorinated alkyl groups may be partially fluorinated, substituted, branched, unsaturated, and/or cyclic. In certain particular embodiments, R₁ is —CO₂R_{A}, wherein R_{A} is fluorinated aryl or fluorinated arylalkyl. In certain particular embodiments, R₁ is —CO₂R_{A}, wherein R_{A} is of the formula:

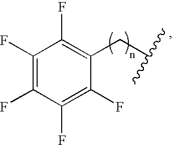

wherein n is an integer between 0 and 12, inclusive. In certain embodiments, n is 0. In certain embodiments, n is 1. In other embodiments, n is 2, 3, 4, 5, or 6. In certain particular embodiments, R₁ is —CO₂R_{A}, wherein R_{A} is of the formula:

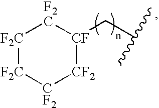

wherein n is an integer between 0 and 12, inclusive. In certain embodiments, n is 0. In certain embodiments, n is 1. In other embodiments, n is 2, 3, 4, 5, or 6.

In certain embodiments, R₂ includes more than 1, 2, 3, 4, 5, 10, 15, 20, or 25 fluorine atoms. In certain embodiments, only one of R₁ and R₂ includes fluorine atoms. In other embodiments, both R₁ and R₂ include fluorine atoms. In certain embodiments, R₂ is fluorine. In other embodiments, R₂ is hydrogen. In other embodiments, R₂ is substituted or unsubstituted, branched or unbranched aliphatic. In yet other embodiments, R₂ is C₁-C₆ alkyl. In certain embodiments, R₂ is a perfluorinated alkyl moiety. In certain particular embodiments, R₂ is methyl. In certain embodiments R₂ is —CF₃, —CHF₂, or —CH₂F. In certain embodiments, R₂ is a fluorine-substituted aryl or heteroaryl moiety. In certain embodiments, R₂ is a fluorine-substituted phenyl moiety. In certain particular embodiments, R₂ is a perfluorinated phenyl moiety.

In certain embodiments, R₁ is —CO₂R_{A}, and R₂ is fluorine. In certain embodiments, R₁ is —CO₂R_{A}, and R₂ is hydrogen. In other embodiments, R₁ is —CO₂R_{A}, and R₂ is methyl. In yet other embodiments, R₁ is —CO₂R_{A}, and R₂ is —CF₃. In certain embodiments, at least one of R₁ and R₂ is fluorine. In other embodiments, both R₁ and R₂ are fluorine.

Exemplary fluorinated alkynyl monomers include:

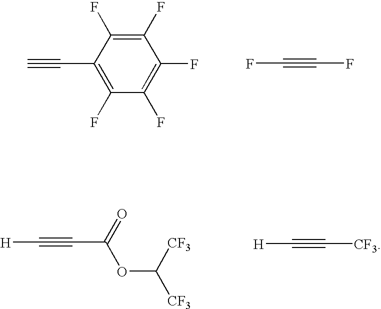

In certain other embodiments, the fluorinated monomer is a fluorinated oligomer. The fluorinated monomers described herein are partially polymerized to form fluorinated oligomers. The fluorinated oligomers are applied to skin and further polymerized on the treated skin. In certain embodiments, the fluorinated oligomers are of a molecular weight sufficient to apply the oligomer to skin. In certain embodiments, the molecular weight of the oligomer is less than 1,000 g/mol. In certain embodiments, the molecular weight is less than 1,500 g/mol. In other embodiments, the molecular weight is less than 2,000 g/mol. In other embodiments, the molecular weight is less than 3,000 g/mol. In other embodiments, the molecular weight is less than 4,000 g/mol. In yet other embodiments, the molecular weight is less than 5,000 g/mol. In certain embodiments, the oligomer is sufficiently large to prevent systemic exposure to the oligomer. For example, in certain embodiments, less than 5%, less than 2%, less than 1%, or less than 0.1% of the applied oligomer reaches the systemic circulation of the subject.

In certain embodiments, the fluorinated monomer is mixed with one or more different monomers. The resulting polymer is a co-polymer. As would be appreciated by those of skill in this art, a co-polymer may have desirable properties not attainable with a polymer resulting from the polymerization of one monomer alone. In certain embodiments, two different monomers are applied to skin. In other embodiments, three different monomers are applied to skin. When different monomer are used, the monomers are applied to skin simultaneously or separately. In certain embodiments, the monomers are all in the same solution which is applied to the skin. In certain embodiments, one of the monomers is fluorinated, and another is not fluorinated. In other embodiments, all monomers are fluorinated.

The monomer can be applied to skin using any method. The skin to be treated is brushed, sprayed, rubbed, smeared, etc. with the monomer or a solution or cosmetic composition of the monomer. In certain embodiments, a cosmetic composition comprising the polymerizable monomer is applied to the skin. In certain embodiments, the monomer is dissolved in a solvent such as water, alcohol, or other solvent and applied to skin. The solvent may include a propellant such as difluoroethane or dimethyl ether. In certain particular embodiments, the initiator is applied to skin simultaneously with the monomer. In other embodiments, the initiator is applied to skin separately from the monomer. In still other embodiments, the initiator is dissolved in the same solution which contains the monomer. Typically, the concentration of monomer ranges from 0.1% to 10%. In certain embodiments, the initiator is at a concentration ranging from 0.1% to 5%. In certain embodiments, the concentration ranges from 0.1% to 3%. In other embodiments, the concentration of initiator ranges from 0.1% to 2%.

The monomer is typically soluble in a variety of organic solvents (e.g. alcohol), propylene glycol, glycerol, water, or aqueous solutions. In certain embodiments, the initiator is soluble in water or an aqueous solution. An aqueous solution may be acid or basic. In certain embodiments, the initiator is soluble in an alcohol (e.g. methanol, ethanol, denatured ethanol, isopropanol, butanol). Examples of other solvents that can be used for the initiators and/or monomer include, but are not limited to, acetic acid, acetone, alcohol, alcohol (denatured), benzophenone, butoxydiglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C9-11 isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopopyl adipate, diifiopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7 diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glyol, neopentyl glyol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut pil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, xylene. In a preferred embodiment, the solvent is selected from the group consisting of propylene glycol, ethanol, isopropanol, n-butanol, water, and mixtures thereof. In certain embodiments, the solvent comprises a mixture of propylene glycol and denatured ethanol. In certain embodiments, the solvent is fluorinated such as 3M Cosmetic Fluid CF-61 or CF-76. As would be appreciated by one of skill in the art, a mixture of more than one solvent in appropriate proportions may be used to deliver the monomer. In certain embodiments, an suspension or emulsion of the monomer is used. In certain embodiments, an emulsifier, detergent, or surfactant is used in the monomer emulsion. In certain embodiments, the surfactant is a fluorinated surfactant (e.g., 3M Novec Fluorosurfactant). In certain embodiments, a propellant is used as at least part of the solvent. Exemplary propellants include difluoroethane and diemthyl ether. In all embodiments, a solvent is optional.

### Polymerization Initiators

The in situ polymerization of the monomers on skin is accomplished via a free radical or ionic polymerization reaction. The polymerization is typically begun using a polymerization initiator. However, in some instances, an initiator may not be used. The polymerization initiator may be chosen based on the type of monomers being used, the type of initiation (e.g. heat or photoinitiation), and solubility of initiator in a solvent or other excipient.

In certain embodiments, the initiator is a free radical initiator, which forms free radicals upon exposure to light or upon heating. Typically, the initiator decomposes upon heating or exposure to a certain wavelength of light to yield two free radicals that initiate the polymerization reaction. The free radical generated from the initiator reacts with an unsaturated functional group (e.g., an alkene, acrylate, or methacrylate functionality) of a monomer thus beginning the chain reaction which results in the formation of the desired polymer.

In certain embodiments, the inventive system takes advantage of oxygen tolerant polymerization initiators. Oxygen-tolerant initiators eliminate the need for an oxygen-free or an oxygen-reduced environment for the polymerization reaction to take place. Such oxygen-tolerant initiators allow for the polymerization reaction to take place directly on skin in a normal atmosphere with about 21% oxygen. Exemplary oxygen tolerant polymerization initiators include 4,4′-azobis(4-cyanovaleric acid); 1,1′-azobis(cyclohexanecarbonitrile); 2,2′-azobis(2-methylpropionitrile); benzoyl peroxide; 2,2-bis(tert-butylperoxy)butane; 2,5-bis(tert-butylperoxy)-2,5-dimethylhexane; bis[1-(tert-butylperoxy)-1-methyl ethyl]benzene; tert-butyl hydroperoxide; tert-butyl peracetate; tert-butyl peroxide; tert-butyl peroxybenzoate; cumene hydroperoxide; dicumyl peroxide; lauroyl peroxide; peracetic acid; potassium persulfate; 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone; and 4-methylbenzophenone.

The initiator is applied to skin in the same ways the monomer is applied to skin. The skin to be treated is brushed, sprayed, rubbed, smeared, etc. with the initiator or a solution or cosmetic composition of the initiator. In certain embodiments, the initiator is dissolved in a solvent such as water, alcohol, or other cosmetically acceptable solvent, and applied to skin. In certain embodiments, a cosmetic composition comprising the polymerization initiator is applied to the skin. In certain particular embodiments, the initiator is applied to skin simultaneously with the monomer. In other embodiments, the initiator is applied to skin separately from the monomer. In this case, the solvent for the monomer may be different that the solvent used for the polymerization initiator. The monomer and initiator may be applied in any order. In still other embodiments, the initiator is dissolved in the same solution which contains the monomer. The initiator is typically at a lower concentration in the solution than the monomer. Typically, the concentration of initiator is approximately 1000-fold, 100-fold, 10-fold, or 5-fold less than the concentration of monomer. In certain embodiments, the initiator is at a concentration ranging from 0.001% to 10%. In certain embodiments, the initiator is at a concentration ranging from 0.001% to 5%. In certain embodiments, the concentration ranges from 0.01% to 1%. In other embodiments, the concentration of initiator ranges from 0.1% to 1%. In certain embodiments, when a high concentration of polymerization initiator is needed, the initiator may be applied neat (i.e., without a solvent).

The initiator is typically soluble in a variety of organic solvents (e.g., alcohol, denatured ethanol, isopropanol), propylene glycol, glycerol, water, or aqueous solutions. Selection of an acceptable solvent will depend on the initiator as well as the method of application. Typically an acceptable solvent will not adversely impact the in situ polymerization process.

In certain embodiments, the initiator is soluble in water or an aqueous solution. An aqueous solution may be acid or basic. In certain embodiments, the initiator is soluble in an alchol (e.g., methanol, ethanol, denatured ethanol, isopropanol, butanol). Examples of other solvents that can be used for the initiators and/or monomer include, but are not limited to, acetic acid, acetone, alcohol, alcohol (denatured), benzophenone, butoxydiglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C9-11 isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopopyl adipate, diisopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7 diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glyol, neopentyl glycol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut pil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, and xylene. In a preferred embodiment, the solvent is selected from the group consisting of propylene glycol, ethanol, isopropanol, n-butanol, water, and mixtures thereof. As would be appreciated by one of skill in the art, a mixture of more than one solvent in appropriate proportions may be used to deliver the initiator(s) and/or monomer(s). In certain embodiments, a propellant such as difluoroethane or dimethyl ether is used as at least part of the solvent. In certain embodiments, the solvent is fluorinated such as 3M Cosmetic Fluid CF-61 or CF-76. In all embodiments, a solvent is optional.

The initiator for the polymerization reaction is typically chosen based on a variety of concerns including the structure of the monomer, toxicity, biocompatibility, solubility, heat versus photo initiation, tolerance to oxygen, tolerance to water, etc. In certain embodiments, the initiator is compatible with initiating polymerization of at least one of the polymerizable monomers to be used in the skin treatment. In certain particular embodiments, the initiator is oxygen tolerant. In certain embodiments, the initiator is non-toxic. In other embodiments, the initiator is biocompatible. In certain embodiments, the initiator is oxygen tolerant. These and other concerns may be taken into account by one of skill in the art choosing the initiator to be used. The initiator may be obtained from a commercial source such as Sigma-Aldrich, Ciba-Geigy, Sartomer, etc. The initiator may also be prepared synthetically.

The inventive system may include the use of one or more polymerization initiators. In certain embodiments, 2, 3, 4, or more polymerization initiators are used. In certain embodiments, one polymerization initiator is used. In certain embodiments, two polymerization initiators are used. In certain embodiments, three polymerization initiators are used. In certain embodiments, more than one initiator is used, and each of the initiators is used to initiate the polymerization of a different monomer being used in the treatment. The different polymerization initiators may be provided for application to skin in different or the same composition with or without monomer.

In certain embodiments, the initiator is a free radical thermal initiator. Any thermal initiator may be used in the polymerization reaction. In certain embodiments, the thermal initiator is designed to work at a temperature ranging from 30° C. to 120° C. In certain embodiments, the initiator is designed to work at a temperature ranging from 30° C. to 100° C. In other embodiments, the initiator is designed to work at a temperature ranging from 30° C. to 80° C. In certain embodiments, the initiator is designed to work at a temperature ranging from 40° C. to 70° C. In certain particular embodiments, the initiator is designed to work at approximately 30, 40, 50, 60, 70, 80, 90, 100, or 110° C. In certain embodiments, a co-initiator is used. Co-initiators act to lower the decomposition temperature of the initiator. Exemplary co-initiators include, but are not limited to, aromatic amine (e.g., dimethyl aniline), organic peroxides, decahydroacridine 1,8-dione, etc. Other co-initiators are list below. The heat may be applied to skin with monomer and initiator applied for about 10 seconds to about 5 minutes. In certain embodiments, the heat is applied for about 10 to about 60 seconds. In other embodiments, the heat is applied for about 10 to about 30 seconds. In yet other embodiments, the heat is applied for about 20 to about 40 seconds. The heat source for initiating polymerization may include, but is not limited, to blow dryers, heat lamps, etc.

Thermal initiators include peroxides, peracids, peracetates, persulfates, etc. Exemplary thermal initiators include tert-amyl peroxybenzoate; 4,4′-azobis(4-cyanovaleric acid); 1,1′-azobis(cyclohexanecarbonitrile); 2,2′-azobis(2-methylpropionitrile); benzoyl peroxide; 2,2′-azo-bis-isobutyronitrile (AIBN); benzoyl peroxide; 2,2-bis(tert-butylperoxy)butane; 1,1-bis(tert-butylperoxy)cyclohexane; 2,5-bis(tert-butylperoxy)-2,5-dimethylhexane; 2,5-bis(tert-butylperoxy)-2,5-dimethyl-3-hexyne; bis[1-(tert-butylperoxy)-1-methylethyl]benzene; 1,1-bis(tert-butylperoxy)-3,3,5-trimethylcyclohexane; tert-butyl hydroperoxide; tert-butyl peracetate; tert-butyl peracetic acid; tert-butyl peroxide; tert-butyl peroxybenzoate; tert-butylperoxy isopropyl carbonate; cumene hydroperoxide; cyclohexanone peroxide; dicumyl peroxide; lauroyl peroxide; 2,4-pentanedione peroxide; peracetic acid; and potassium persulfate. Many of the above listed thermal initiators are available from commercial sources such as Sigma-Aldrich. In certain embodiments, the initiator is 2,2′-azo-bis-isobutyronitrile (AIBN). In other embodiments, the initiator is benzoyl peroxide (also known as dibenzoyl peroxide). In certain embodiments, a combination of thermal initiators is used. In certain embodiments, the polymerization initiator is a combination of ammonium persulfate (APS) and N,N,N′,N′-tetramethylethylenediamine (TEMED).

In other embodiments, the free radical initiator is a photoinitiator. Photoinitiators produce reactive free radical species that initiate the polymerization of monomers upon exposure to light. Any photoinitiator may be used in the polymerization reaction. Photoinitiated polymerizations and photoinitiators are discussed in detail in Rabek, Mechanisms of Photophysical Processes and Photochemical Reactions in Polymers, New York: Wiley & Sons, 1987; Fouassier, Photoinitiation, Photopolymerization, and Photocuring, Cincinnati, Ohio: Hanser/Gardner; Fisher et al., “Photoinitiated Polymerization of Biomaterials” Annu. Rev. Mater. Res. 31:171-81, 2001; incorporated herein by reference. The photoinitiator may be designed to produce free radicals at any wavelength of light. In certain embodiments, the photoinitiator is designed to work using UV light (200-500 nm). In certain particular embodiments, the photoinitator is designed to work using UV light with a wavelength of approximately 365 nm. In certain embodiments, long UV rays are used. In other embodiments, short UV rays are used. In other embodiments, the photoinitiator is designed to work using visible light (400-800 nm). In certain embodiments, the photoinitiator is designed to work using blue light (420-500 nm). In yet other embodiments, the photoinitiator is designed to work using IR light (800-2500 nm). The output of light can be controlled to provide greater control over the polymerization reaction. Control over the polymerization reaction in turn results in control over the skin treatment. In certain embodiments, the intensity of light ranges from about 500 to about 10,000 μW/cm². In certain embodiments, the intensity of light is about 4000, 5000, 6000, 7000, 8000, or 9000 μW/cm². The light may be applied to skin with monomer and initiator applied for about 10 seconds to about 5 minutes. In certain embodiments, the light is applied for about 10 to about 60 seconds. In other embodiments, the light is applied for about 10 to about 30 seconds. In yet other embodiments, the light is applied for about 20 to about 40 seconds. The light source may allow variation of the wavelength of light and/or the intensity of the light. Light sources useful in the inventive system include, but are not limited to, lamps, fiber optics devices, etc.

In certain embodiments, the photoinitiator is a peroxide (e.g., ROOR′). In other embodiments, the photoinitiator is a ketone (e.g. RCOR′). In other embodiments, the compound is an azo compound (e.g., compounds with a —N═N— group). In certain embodiments, the photoinitiator is an acylphosphineoxide. In other embodiments, the photoinitiator is a sulfur-containing compound. In still other embodiments, the initiator is a quinone. Exemplary photoinitiators include acetophenone; anisoin; anthraquinone; anthraquinone-2-sulfonic acid, sodium salt monohydrate; (benzene) tricarbonylchromium; 4-(boc-aminomethyl)phenyl isothiocyanate; benzin; benzoin; benzoin ethyl ether; benzoin isobutyl ether; benzoin methyl ether; benzoic acid; benzophenone; benzyl dimethyl ketal; benzophenone/1-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diethylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizer (Biddle Sawyer); Irgacure 907 (Ciba Geigy); Irgacure 651 (Ciba Geigy); Darocur 2959 (Ciba Geigy); ethyl-4-N,N-dimethylaminobenzoate (4EDMAB); 1-[-(4-benzoylphenylsulfanyl)phenyl]-2-methyl-2-(4-methylphenylsulfonyl) propan-1-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-1-phenyl-1-propanone; 65% (oligo[2-hydroxy-2-methyl-1-[4-(1-methylvinyl)phenyl]propanone] and 35% propoxylated glyceryl triacrylate; benzil dimethyl ketal; benzophenone; blend of benzophenone and a-hydroxy-cyclohexyl-phenyl-ketone; blend of Esacure KIP 150 and Esacure TZT; blend of Esacure KIP 150 and Esacure TZT; blend of Esacure KIP 150 and TPGDA; blend of phosphine oxide, Esacure KIP 150 and Esacure TZT; difunctional a-hydroxy ketone; ethyl 4-(dimethylamino) benzoate; isopropyl thioxanthone; 2-hydroxy-2-methyl-phenylpropanone; 2,4,6,-trimethylbenzoyldiphenyl phosphine oxide; 2,4,6-trimethyl benzophenone; liquid blend of 4-methylbenzophenone and benzophenone; oligo(2-hydroxy-2 methyl-1-(4 (1-methylvinyl)phenyl)propanone; oligo(2-hydroxy-2-methyl-1-4 (1-methylvinyl)phenyl propanone and 2-hydroxy-2-methyl-1-phenyl-1-propanone (monomeric); oligo (2-hydroxy-2-methyl-1-4 (1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone. In certain embodiments, the photoinitiator is acetophenone; diphenyl(2,4,6-trimethylbenzoyl)phosphine oxide; 4,4′-dimethoxybenzoin; anthraquinone; anthraquinone-2-sulfonic acid; benzene-chromium(0) tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1-hydroxycyclohexyl phenyl ketone, 50/50 blend; benzophenone-3,3′,4,4′-tetracarboxylic dianhydride; 4-benzoylbiphenyl; 2-benzyl-2-(dimethylamino)-4′-morpholinobutyrophenone; 4,4′-bis(diethylamino)benzophenone; Michler's ketone; (±)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4-dimethylbenzophenone; diphenyl(2,4,6-trimethylbenzoyl)phosphine oxide/2-hydroxy methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts; 3-mercapto-1-propanol; 11-mercapto-1-undecanol; 1-mercapto-2-propanol; and 3-mercapto-2-butanol, all of which are commercially available from Sigma-Aldrich. In certain embodiments, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone; and 4-methylbenzophenone. In certain embodiments, the photoinitiator is dimethoxy-2-phenyl-acetophenone (DMPA). In certain embodiments, the photoinitiator is a titanocene. In certain embodiments, a combination of photoinitiators is used.

In other embodiments, an initiator of a cationic or anionic polymerization process is used. In certain embodiments, the initiator is a photoinitiator of a cationic polymerization process. Exemplary photoinitiators of cationic polymerization include, but are not limited to, titanium tetrachloride, vanadium tetrachloride, bis(cyclopentadienyl)titanium dichloride, ferrocene, cyclopentadienyl manganese tricarbonyl, manganese decacarbonyl, diazonium salts, diaryliodonium salts (e.g. 3,3′-dinitrodiphenyliodonium hexafluoroarsenate, diphenyliodonium fluoroborate, 4-methoxydiphenyliodonium fluoroborate) and triarylsulfonium salts. In certain embodiments, a hybrid free radical/cationic photopolymerization is used to polymerize the monomers in situ on skin.

### Cosmetic Compositions

The present invention also provides cosmetic compositions comprising a polymerizable monomer and/or a polymerization initiator, and a cosmetically suitable vehicle. The cosmetic composition is formulated for application to the skin of a subject (e.g., a human). The cosmetic composition may be a cream, a lotion, a solution, an ointment, an emulsion, a powder, a spray, a foam, a gel, or other composition suitable for application to the skin. In certain embodiments, the vehicle allows for the easy application of the polymerizable monomer and/or a polymerization initiator to the skin. The vehicle may include emollients which lubricate or hydrate the skin. The cosmetic composition may in addition to a polymerizable monomer and/or a polymerization initiator also include an active ingredient such as a vitamin, anti-inflammatory agent, retinoid, anti-oxidant, steroid, caffeine, sunscreen, protein, peptide, carbohydrate, lipid, polynucleotide, or other biologically active agent. The composition may also include a preservative, a coloring agent, a pigment, a dye, an optical agent, or a fragrance.

Any polymerizable monomer and/or a polymerization initiator may be utilized in the inventive cosmetic compositions. In certain embodiments, the polymerizable monomer and/or a polymerization initiator is described herein.

The amount of each of the polymerizable monomer and/or a polymerization initiator in the composition may range from approximately 0.001% to approximately 50% by weight of the composition. In certain embodiments, the amount of the polymerizable monomer or polymerization initiator is between approximately 0.01% and approximately 20%. In certain embodiments, the amount of the polymerizable monomer or polymerization initiator is between approximately 0.01% and approximately 1% by weight. In certain embodiments, the amount of the polymerizable monomer or polymerization initiator is between approximately 0.001% and approximately 0.1% by weight. In certain embodiments, the amount of the polymerizable monomer or polymerization initiator is between approximately 0.5% and approximately 10% by weight. In certain embodiments, the amount of the polymerizable monomer or polymerization initiator is between approximately 1% and approximately 5% by weight. In certain embodiments, the amount of the polymerizable monomer or polymerization initiator in the composition is approximately 0.001%, 0.005%, 0.01%, 0.05%, 0.1%, 0.25%, 0.5%, 0.75%, 1%, 2%, 3%, 4%, 5%, 6%, 7%, 8%, 9%, or 10%.

The remainder of the composition besides the polymerizable monomer and/or polymerization initiator is typically a cosmetically suitable vehicle. Active ingredients may also be included in the inventive compositions (e.g. sunscreen (derivatives of PABA, cinnamates, salicylates, etc.), steroids, retinoids, anti-inflammatory agents, vitamins (vitamin A, vitamin E, biotin, vitamin C, vitamin B₃, vitamin F, D-panthenol, etc.), antibiotics, etc.), antioxidants, proteins, peptides, polynucleotides, carbohydrates, and other bioactive agents. In certain embodiments, the compositions further comprise a plant extract (e.g., St. John's wort extract, witch hazel extract, chamomile extract, arnica extract, ginseng extract, aloe vera, green tea extract, white tea extract, etc.), coloring agent (e.g., natural and artificial pigments), fragrance, protein (e.g. tropoelastin, collagen, elastin, procollagen, fibronectin, etc.), peptide, polynucleotide, etc.

Various non-toxic, dermatologically acceptable vehicles in which the polymerizable monomer and/or polymerization initiator are stable are available in the art. In general, lubricating vehicles which help hydrate the skin are preferred. Various cosmetically acceptable vehicles are described in U.S. Pat. Nos. 7,118,735; 7,083,780; 7,067,140; 7,001,604; 6,979,452; 6,919,072; 6,864,274; 6,790,434; 6,759,052; 6,682,749; 6,630,516; 6,451,339; 6,261,603; 6,238,284; 6,146,650; 5,922,331; 5,837,224; 5,747,051; 5,322,685; 5,254,331; 5,153,230; 4,877,805; 4,801,586; and 4,228,162; each of which is incorporated herein by reference. Cosmetically acceptable vehicles are also described in the following international and foreign patent references: WO2005/097068; WO 2004/016289; WO 89/04179; DE 3442402; EP-A-131927; GB 2139496; GB 2146525; E-A 120262; DD 217989; JP-A-60-64418; each of which is incorporated herein by reference. Any of the vehicles described herein or in the cited references may be combined to form mixtures that act as the vehicle in the inventive compositions.

In certain embodiments, the composition comprises an emulsifier as part of the cosmetically suitable vehicle. The emulsifier may be an anionic, cationic, or neutral emulsifier. In certain embodiments, the emulsifier is an anionic emulsifier selected from the group consisting of alkyl sulphate, aralkyl sulphates, alkyl ethoxy ether sulphates, alkaryl sulphonates, alkyl succinates, alkyl sulphosuccinates, N-alkoyl sarconsinates, isethionates, N-acyl taurate, sodium lauryl sulfate, sodium laureth sulfate, sodium oleyl succinate, sodium dodecylbenzenesulfonate, and sodium lauryl sarconsinate. Exemplary non-ionic or neutral emulsifiers include sorbitan ester, ethoxylated sorbitan ester, ethoxylated alkyl ether, ethoxylated fatty acid ether, fatty alcohol, ethoxylated fatty alcohol, and esters of glycerin and fatty acids. In certain embodiments, the emulsifiers are synthetic or natural polymers.

In certain embodiments, the composition comprises an oil, lipid, wax, fatty alcohols, glycerides, or fatty acid as part of the cosmetically suitable vehicle. Exemplary oils that may be used in the composition include triglycerides, diglycerides, monoglycerides, cholesterol, lanosterol, lanolin oil, cetyl alcohol, stearyl alcohol, cetyl ester wax, cod liver oil, soybean oil, fish liver oil, squalene, liquid paraffin, ceresin oil, 2-octyldodecanol, 2-hexyldecanol, crotamiton, 1-menthol, mentha oil, benzyl alcohol, silicone oil, white petrolatum, corn oil, avocado oil, sesame oil, etc. In certain embodiments, the composition comprises a fatty acid selected from the group consisting of salts and esters of palmitate, salts and esters of stearate, salts and esters of laurate, salts and esters of oleate, isopropyl myristate, isopropyl palmitate, cis-oleic acid, diisopropyl sebacate, diethyl sebacate, diisopropyl adipate, glycerol caprate, linoleic acid, γ-linolenic acid, homo-γ-linolenic acid, columbinic acid, eicosa-n-6,9,13)-trienoic acid, arachidonic acid, γ-linolenic acid, timnodonic acid, hexaenoic acid, sorbitan sesquioleate, polyoxyl 40 stearate, glycerol caprylate, myristyl myrisate, myristyl palmitate, myristyl stearate, myristyl isostearate, myristyl oleate, myristyl behenate, myristyl erucate, cetyl myristate, cetyl palmitate, cetyl sterate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate, and erucyl erucate. In certain embodiments, the lipid is a naturally occurring lipid. In certain embodiments, the lipid is a phospholipid. In certain embodiments, the lipid is a glycosphingolipid. Exemplary waxes include beeswax, carnauba wax, candelilia wax, ouricuri wax, Japan wax, esparto grass wax, shellac wax, spermaceti, lanolin (wool wax), petrolatum, uropygial grease, guaruma wax, cork fibre wax, sugarcane wax, rice wax, montan wax, paraffin, lignite wax, microcrystalline wax, ceresin, ozokerite, polyethylene wax, Fischer-Tropsch waxes, octacosanyl stearate, glycerides, silicone waxes, and poly(di)methylsiloxane esters. Exemplary alcohols include lauryl alcohol, coconut fatty alcohol, myristyl alcohol, cetyl alcohol, cetearyl alcohol, stearyl alcohol, isostearyl alcohol, oleyl alcohol, elaidyl alcohol, petroselinyl alcohol, linolyl alcohol, and linolenyl alcohol.

In certain embodiments, the composition comprises a carbohydrate as part of the cosmetically suitable vehicle. Exemplary carbohydrates include monosaccharides, disaccharides, oligosaccharides, and polysaccharides. Exemplary polysaccharides include cellulose, methylcellulose, hydroxypropylmethylcellulose, chitin, galactoarabinan, polygalactose, and polyarabinose. Exemplary glycerides includes hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartartic acid monoglyceride, tartartic acid diglyceride, citric acid monoglyceride, citric acid diglyceride, malic acid monoglyceride, malic acid monoglyceride, malic acid diglyceride, and mixture thereof.

In certain embodiments, the composition comprises a polymer or thickening agent. The polymer may be a natural or synthetic polymer. Natural polymers include polysaccharides, nucleic acid, and proteins. Synthetic polymers include polyesters, polyureas, polycarbonates, polyvinyl alcohol, polyamides, polyethers, polyesters, polyamines, polytyrosines, polyanhydrides, polyphosphazenes, polyacrylamides, polyacrylates, polymethacrylates, polyvinylpyrrolidone, etc. Exemplary thickening agents include alginate derivatives, preneutralized carbomer 430, hydrophilic silicas, polysaccharides, xanthan gum, guar guar, agar agar, carboxymethylcellulose, hydroxyethylcellulose, polyacrylates, polyacrylamides, polyvinylpyrrolidone, and salts.

In certain embodiments, the cosmetically suitable vehicle includes a solvent. In certain embodiments, the solvent comprises water. In certain embodiments, the solvent comprises an alcohol (e.g., methanol, ethanol, isopropanol, butanol, tert-butanol, etc.). In certain embodiments, the solvent comprises propylene glycol, butylene glycol, butylated hydroxytoluene, or glycerin. In certain embodiments, the solvent is dimethylisosorbide. In certain embodiments, the solvent is 3,6-dimethoxyfuro[3,2-b]furan. In certain embodiments, the solvent is propylene glycol.

In certain embodiments, the composition further comprises a preservative. In certain embodiments, the preservative is quaternium-15, methylparaben, propylparaben, or diazolidinyl urea. In certain embodiments, the preservative is a metal chelating agent. The metal chelating agent binds metal ions that might accelerate the degradation of composition. In certain embodiments, the chelating agents is EDTA (e.g., disodium EDTA, tetrasodium EDTA, or other salts of EDTA), citric acid or a salt thereof, tartaric acid or a salt thereof, organo aminophosphonic acid (e.g., tri(methylene phosphonic acid), diethylene triamine penta(methylene phosphonic acid), hexamethylene diamine tetra(methylene phosphonic acid), etc.), organo phosphonic acids, nitrilotriacetic acid, polyaminocarboxylic acids (e.g., ethylenetriamine pentacetic acid), and iminodiacetic acids (e.g. 2-hydroxyl diacetic acid, glycerol imino diacetic acid). In certain embodiments, the preservative is an anti-oxidant such as butylated hydroxytoluene (BHT), vitamin E, derivatives of vitamin E, vitamin C, derivatives of vitamin C, and sodium metabisulfite. Various combinations of the preservatives described herein may also be used in the inventive compositions.

### Polymerization Reaction and Use

The monomer(s), initiator(s), or compostions thereof as discussed above are applied to skin to be treated using the inventive system. The monomers are then polymerized on the skin using light or heat to initiate the polymerization reaction. The amount of light and heat, as described above, will depend on the monomers and initiator being used, the skin being treated, concentration of the initiator, concentration of the monomer, etc. Basic guidelines are provided herein for the inventive system using various initiator; however, these guidelines may be adjusted by one of skill in the art to provide the desired results.

According the methods of the invention, the skin to be treated is optionally washed first to remove any excess dirt or oil before the treatment is begun. The monomer and polymerization initiator or a composition thereof is then applied to the skin by any technique known in the art including spraying, brushing, rubbing, smearing, rolling-on, immersing, dipping, spattering, pouring, etc. The monomer and polymerization initiator is not injected into or below the skin. The treatment may be applied to a portion of the skin. For example, the treatment may be applied to only the head and neck. In certain embodiments, the treatment may be applied to only exposed skin such as face, neck, hands, arms, legs, etc. As described above, the monomer and polymerization initiator may be applied together or separately. The compositions for application to skin may include some or all of the following properties: good consistency, good distributability, economical application, good definition and texture, slight load, good strength, lack of undesired residue, and suitable drying time. After both have been applied to the skin, the skin is exposed to light or heat to initiate the in situ polymerization process. In certain embodiments, the monomers are polymerized concomitantly with the application of the monomer and initiator. In certain embodiments, the monomers are polymerized both concomitantly with application of the monomer and initiator and subsequent to the application. In certain particular embodiments, the composition(s) being applied to the skin provides the heat needed to initiate the polymerization reaction by an exothermic reaction. In other embodiments, the skin is allowed to dry before the polymerization reaction is begun. In other embodiments, the polymerization is started as soon as the monomer is applied to the skin. In certain embodiments, the application and polymerization steps are repeated until the desired skin characteristic is achieved. In certain embodiments, the polymerization process results in a branched or cross-linked polymer which results in a stronger polymer. Such a polymer may provide better bonding to the skin. The inventive system may be used to produce a desired cosmetic effect. In certain embodiments, the desired characteristic is shine, smoothness, feel, etc. In certain embodiments, the desired characteristic reduces the appearance of aging (e.g., reducing the appearance of lines or wrinkles). Without wishing to be bound by a particular theory, the polymerization on the surface of the skin is thought to reduce the appearance of lines and wrinkles by physically tightening the skin during the polymerization process. In certain embodiments, the inventive skin care system is used to provide a protective effect from UV light. In other embodiments, the inventive system is used to give skin a distinct feel. In certain embodiments, the treatment is used to color skin. For example, pigments may be associated with the polymer film formed on the skin. In other embodiments, the treatment is used to restore damaged skin (e.g., sun damaged skin). In certain embodiments, the treatment is used to help exfoliate the skin. For example, the polymeric film may be used in skin peels to help exfoliate the skin. In certain other embodiments, the treatment may be used to lighten the skin or make the skin appear lighter. In certain embodiments, the treatment may be used to darken the skin or make the skin appear darker.

Moreover, instead of or in addition to simply imparting and maintaining a cosmetic effect, the composition applied to skin may include dyes or pigments, thereby resulting in coloring the skin. The dyes or pigments may be covalently associated with the components of the composition such as the monomers. In such case, the dye or pigment may become part of the polymer. In other embodiments, the dye or pigment is separate but may become entrapped in the polymeric matrix formed on the surface of the skin. In certain embodiments, the pigments used are those typically found in make-up and other cosmetics. Furthermore, other compounds conducive to skin treatment may be used in the inventive system. For example, vitamins, and lipids may be included in the composition applied to skin. In certain embodiments, the inventive system is used to deliver agents that enhance the health of skin. In certain embodiments, the inventive system is used to deliver agents that enhance the elasticity of skin. In certain embodiments, the inventive system is used to deliver agents known in the art to enhance the optical properties of skin (e.g., shine, color). In certain embodiments, the inventive system is used to deliver agents known in the art to change the feel of skin.

The inventive system may be used on any animal. The system is particularly useful for treating human skin. However, the skin of other mammals may also be treated. In addition, the skin of animals such as rodents (e.g., mouse, rat, rabbit, guinea pig, etc.) or primates may also be treated.

The in situ polymerization process can be initiated by a light or heat source. In certain embodiments, a light source is used. The light source may be an IR, visible, or UV light source. The wavelength(s) of light generated by the light source should typically correspond with the wavelength of light for activating the polymerization initiator used. The light source may allow for generation of light of varying wavelengths and intensity. Varying the output of light allows for greater control of the polymerization process.

In certain embodiments, the light source is an IR light source. In other embodiments, the light source is a visible light source. In still other embodiments, the light source is a UV light source. In certain embodiments, the light source emits light with a wavelength of about 200 nm to about 600 nm and an intensity of about 500 μW/cm² to about 10,000 μW/cm². In certain particular embodiments, the light source emits light at a wavelength of 365 nm and at an intensity of about 7,000 μW/cm². In certain embodiments, the light source emits light at an intensity of about 4000, 5000, 6000, 7000, 8000, or 9000 μW/cm². In certain embodiments, the light source emits light at a wavelength of about 200 to about 400 nm. The light may be applied to the skin concurrently with the application of monomer and/or polymerization initiator and/or subsequent to application of monomer and/or polymerization initiator. The treated skin is exposed to the light source from 5 seconds to 60 seconds. In certain embodiments, the exposure is about 10 seconds to about 30 seconds. In certain embodiments, the exposure is about 20 seconds to about 40 seconds. In certain embodiments, the exposure is about 30 seconds. In certain embodiments, the exposure is about 60 seconds. In certain embodiments, the light source is a globally applied UV light from a source such as a tanning bed or tanning booth. In certain embodiments, a tanning booth or tanning bed may be used to apply by light and heat for activating the polymerization reaction.

In certain embodiments, a heat source is used to initiate the in situ polymerization process. Examples of heat sources that may be used include blow dryers and heat lamps. The output temperature of the heat source is typically in the range of about 50° C. to about 500° C. In certain embodiments, the output temperature of the heat source is from about 50° C. to about 200° C. In certain embodiments, the output temperature of the heat source is from about 50° C. to about 100° C. The heat source may heat the skin to a temperature of about 30° C. to about 80° C. In certain embodiments, the temperature is about 40° C. to about 70° C. In certain embodiments, the temperature is about 45° C. to about 80° C. In certain embodiments, the temperature is about 40° C. to about 50° C. In certain embodiments, the temperature is about 50° C. to about 60° C. In certain embodiments, the temperature is about 50° C. to about 70° C. In certain embodiments, the temperature is about 60° C. to about 80° C. In certain embodiments, the temperature is about 70° C. to about 80° C.

The treated skin is exposed to the heat source from 5 seconds to 120 seconds. In certain embodiments, the exposure is about 10 seconds to about 60 seconds. In certain embodiments, the exposure is about 20 seconds to about 60 seconds. In certain embodiments, the exposure is about 30 seconds. In certain embodiments, the exposure is about 60 seconds. In certain embodiments, the exposure is about 90 seconds. In certain embodiments, the exposure is about 120 seconds. In certain embodiments, the heat source is an exothermic chemical reaction. For example, the exothermic reaction may occur when two compositions applied to the skin come in contact. Or, to give another example, an agent in the composition comes in contact with oxygen or moisture.

Without wishing to be bound by any particular theory, the polymerization reaction is thought to cause the polymerization of the monomers on the skin of the subject being treated. The polymerization reaction may also lead to the covalent attachment of polymer to the skin (e.g., keratin, elastin, collagen, other proteins, lipids, or carbohydrates found in skin). The formed polymer may fill in gaps, cracks, ridges, holes, splits, pits, crevices, lines, wrinkles, etc. on the skin. The resulting polymeric film on the surface of the skin is very thin and does not substantially penetrate the outer layer of the skin. In certain embodiments, the polymeric film may penetrate the stratum corneum. In certain embodiments, the polymeric film may penetrate the stratum corneum and stratum granulsom. In certain embodiments, the polymeric film may penetrate the stratum corneum, stratum granulsom, and stratum spinosum. In certain embodiments, the polymeric film may penetrate the stratum corneum, stratum granulsom, stratum spinosum, and stratum basale. In certain embodiments, the film penetrates the epithelium but not the dermis. In certain other embodiments, the film penetrates the epithelium and the dermis. In certain embodiments, the film is less than approximately 0.1 mm in thickness. In certain embodiments, the film is less than approximately 0.01 mm in thickness. In certain embodiments, the film is less than approximately 0.001 mm in thickness. In certain embodiments, the film is 0.1 to 10 microns in thickness. In certain embodiments, the film is 1 to 10 microns in thickness. The inventive system is particularly useful for treating skin with polymers that could not otherwise be applied to skin using conventional means because of solubility or formulation issues.

The inventive system is not intended to be used for drug delivery or for wound closing or healing. The polymeric film is not intended to be a polymeric depot of a drug for extended or timed release. The polymer is also not formed in the skin or below the skin. Although the polymeric film may extend slightly into the upper layer of the skin from the outside, it is not intended to penetrate the surface of the skin substantially. The inventive system is also not intended to be used for wound closing or stopping bleeding using a surgical glue (e.g., a cyanoacrylate glue).

### Kits

The invention also provides kits for use in treating skin based on the inventive system for the in situ polymerization of polymerizable monomers on the surface of skin. The kit may include all or a portion of the components necessary to treat skin. In certain embodiments, the kit includes enough product for one application. In other embodiments, the kit include enough product for multiple applications (e.g., approximately 2, 3, 4, 5, 10, 15, 20, 25, or 50 applications). The kit may include any or all of the following components: monomers or compositions thereof, photoinitiators or compositions thereof, thermal initiators, solvent (e.g., ethanol, denatured ethanol, propylene glycol), water, vials, heat source, light source, spray bottle, brush, containers, and instructions for use. The compositions of the kit may be packaged as lotions, creams, solutions, gels, emulsions, suspensions, sprays, aerosol sprays, and non-aerosol sprays (e.g., atomisers). Compositions of the kit such as monomer and/or initiator compositions are typically conveniently packaged in a suitable container for shipping and/or application of the composition. For example, a monomer composition may be provided in a pump spray bottle, spray can, cream, gel, or lotion. In certain embodiments, the components of the kits are conveniently packaged for use by the end use along with instructions for use in accordance with the present invention. The kit may or may not include a heat source or light source. In certain embodiments, the kit is tailored for producing a desired characteristic on the treated skin. The kit may also include other skin care products including moisturizers, pigments, etc.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of treating skin, the method comprising steps of:
applying a polymerizable monomer to skin of a subject;
applying a polymerization initiator to skin of a subject; and
polymerizing the monomers in situ on the surface of the skin by activating the polymerization initiator.

2. The method of claim 1, wherein the subject is human.

3. The method of claim 1, wherein the subject is a non-human mammal.

4. The method of claim 1, wherein the treatment results in a polymer that is resistant to humidity, washing, smearing, flaking, or other deterioration of the cosmetic effect.

5. The method of claim 1, wherein the treatment diminishes the appearance of wrinkles, aging, or skin imperfections.

6. The method of claim 1, wherein the treatment protects the treated skin from UV light.

7. The method of claim 1, wherein the method further comprises the step of applying a pigment to the skin of a subject.

8. The method of claim 1, wherein the treatment method is not used for drug delivery.

9. The method of claim 1, wherein the treatment method is not used for wound closing or wound healing.

10. The method of claim 1, wherein the polymerization initiator is activated by irradiation with light.

11. The method of claim 1, wherein the polymerization initiator is activated by irradiation with UV light.

12. The method of claim 10, wherein the light has a wavlength of about 200 nm to about 600 nm.

13. The method of claim 11, wherein the UV light has a wavelength of about 200 to about 400 nm.

14. The method of claim 11, wherein the UV light has a wavelength of about 365 nm.

15. The method of claim 10, wherein the light has an intensity of about 500 μW/cm² to about 10,000 μW/cm².

16. The method of claim 10, wherein the light has an intensity of about 7,000 μW/cm².

17. The method of claim 10, wherein the light is applied for about 10 seconds to about 60 seconds.

18. The method of claim 10, wherein the light is applied concurrently with application of monomer or polymerization initiator to the skin.

19. The method of claim 10, wherein the light is applied subsequent to application of monomer or polymerization initiator to the skin.

20. The method of claim 1, wherein the polymerization initiator is activated by exposure to a heat source.

21. The method of claim 1, wherein the polymerizable monomer comprises a vinyl, acrylate, methacrylate, diene, maleimide, or epoxide moiety.

22. The method of claim 1, wherein the polymerizable monomer is selected from the group consisting of ethyl acrylate, vinyl acrylate, 1,3-butanediol diacrylate, dipentaneerythritol pentaacrylate, tridecyl methacrylate, styrene, 3,4-epoxycyclohexylmethyl 3′,4′-epoxycyclohexane carboxylate, tricyclodecane dimethanol diacrylate, tricyclodecane dimethanol dimethacrylate, polyisoprene di(meth)acrylate, and polybutadiene di(meth)acrylate oligomers.

23. The method of claim 1, wherein the polymerizable monomer has a molecular weight of less than about 2,000 g/mol.

24. The method of claim 1, wherein the polymerizable monomer has a molecular weight of less than about 1,000 g/mol.

25. The method of claim 1, wherein the polymerizable monomer has a molecular weight of less than about 500 g/mol.

26. The method of claim 1, wherein the polymerizable monomer has a molecular weight of greater than about 2,000 g/mol.

27. The method of claim 1, wherein the polymerizable monomer has a molecular weight of greater than about 3,000 g/mol.

28. The method of claim 1, wherein the polymerizable monomer has a molecular weight of greater than about 5,000 g/mol.

29. The method of claim 1, wherein the polymerization initiator is a free radical initiator.

30. The method of claim 1, wherein the polymerization initiator is oxygen-tolerant.

31. The method of claim 1, wherein the polymerization initiator is a thermal initiator.

32. The method of claim 31, wherein the thermal initiator is selected from the group consisting of tert-amyl peroxybenzoate; 4,4′-azobis(4-cyanovaleric acid); 1,1′-azobis(cyclohexanecarbonitrile); 2,2′-azobis(2-methylpropionitrile); benzoyl peroxide; 2,2′-azo-bis-isobutyronitrile (AIBN); benzoyl peroxide; 2,2-bis(tert-butylperoxy)butane; 1,1-bis(tert-butylperoxy)cyclohexane; 2,5-bis(tert-butylperoxy)-2,5-dimethylhexane; 2,5-bis(tert-butylperoxy)-2,5-dimethyl-3-hexyne; bis[1-(tert-butylperoxy)-1-methylethyl]benzene; 1,1-bis(tert-butylperoxy)-3,3,5-trimethylcyclohexane; tert-butyl hydroperoxide; tert-butyl peracetate; tert-butyl peracetic acid; tert-butyl peroxide; tert-butyl peroxybenzoate; tert-butylperoxy isopropyl carbonate; cumene hydroperoxide; cyclohexanone peroxide; dicumyl peroxide; lauroyl peroxide; 2,4-pentanedione peroxide; peracetic acid; and potassium persulfate.

33. The method of claim 1, wherein the polymerization initiator is photoinitiator.

34. The method of claim 33, wherein the photoinitiator is selected from the group consisting of acetophenone; anisoin; anthraquinone; anthraquinone-2-sulfonic acid, sodium salt monohydrate; (benzene)tricarbonylchromium; 4-(boc-aminomethyl)phenyl isothiocyanate; benzin; benzoin; benzoin ethyl ether; benzoin isobutyl ether; benzoin methyl ether; benzoic acid; benzophenone; benzyl dimethyl ketal; benzophenone/1-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diethylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizer (Biddle Sawyer); Irgacure 907 (Ciba Geigy); Irgacure 651 (Ciba Geigy); Darocur 2959 (Ciba Geigy); ethyl-4-N,N-dimethylaminobenzoate (4EDMAB); 1-[-(4-benzoylphenylsulfanyl)pheny 1]-2-methy 1-2-(4-methylphenylsulfonyl)propan-l-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-l-phenyl-1-propanone; 65% (oligo[2-hydroxy-2-methy 1-1-[4-(1-methylvinyl)phenyl]propanone] and 35% propoxylated glyceryl triacrylate; benzil dimethyl ketal; benzophenone; blend of benzophenone and a-hydroxy-cyclohexyl-phenyl-ketone; blend of Esacure KIP150 and Esacure TZT; blend of Esacure KIP150 and Esacure TZT; blend of Esacure KIP150 and TPGDA; blend of phosphine oxide, Esacure KIP150 and Esacure TZT; difunctional a-hydroxy ketone; ethyl 4-(dimethylamino)benzoate; isopropyl thioxanthone; 2-hydroxy-2-methyl-phenylpropanone; 2,4,6,-trimethylbenzoyldiphenyl phosphine oxide; 2,4,6-trimethyl benzophenone; liquid blend of 4-methylbenzophenone and benzophenone; oligo(2-hydroxy-2 methyl-1-(4 (1-methylvinyl)phenyl)propanone; oligo(2-hydroxy-2-methyl-1-4 (1-methylvinyl)phenyl propanone and 2-hydroxy-2-methyl-1-phenyl-1-propanone (monomeric); oligo (2-hydroxy-2-methyl-1-4 (1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone.

35. The method of claim 1, wherein the polymerization initiator is selected from the group consisting of benzophenone, benzyl dimethyl ketal, trimethylphosphine oxides, and methyl thio phenyl morpholino ketones.

36. The method of claim 1, wherein the polymerization initiator is benzoyl peroxide.

37. The method of claim 1, wherein the polymerization initiator is 2,2′-azobisisobutyrylnitrile (AIBN).

38. The method of claim 1, wherein the polymerization initiator is a combination of ammonium persulfate (APS) and N,N,N′,N′-tetramethylethylenediamine (TEMED).

39. The method of claim 1, wherein the polymerizable monomer or polymerization initiator is applied using a solvent.

40. The method of claim 39, wherein the solvent is propylene glycol, denatured ethanol, water, or mixtures thereof.

41. The method of claim 39, wherein the solvent is a propellant.

42. The method of claim 41, wherein the propellant is selected from the group consisting of difluoroethane and dimethyl ether.

43. The method of claim 1, wherein the polymerizable monomer or polymerization initiator is applied as a cream, solution, lotion, gel, foam, or emulsion:

44. The method of claim 1, wherein the step of applying a polymerizable monomer comprises spraying, brushing, dipping, soaking, smearing, or rubbing a composition comprising the monomer on skin of a subject.

45. The method of claim 44, wherein the composition further comprises a polymerization initiator.

46-151. (canceled)
